SEQUENCE LISTING (699 sequences)

SEQ ID NO: 1 - protein, 5 aa, synthetic construct
  REGION 1..5: note = Synthetic Polypeptide
NYDMA

SEQ ID NO: 2 - protein, 17 aa, synthetic construct
  REGION 1..17: note = Synthetic Polypeptide
SIITSGDATYYRDSVKG

SEQ ID NO: 3 - protein, 12 aa, synthetic construct
  REGION 1..12: note = Synthetic Polypeptide
HDYYDGSYGFAY

SEQ ID NO: 4 - protein, 11 aa, synthetic construct
  REGION 1..11: note = Synthetic Polypeptide
KASQSVGINVD

SEQ ID NO: 5 - protein, 7 aa, synthetic construct
  REGION 1..7: note = Synthetic Polypeptide
GASNRHT

SEQ ID NO: 6 - protein, 9 aa, synthetic construct
  REGION 1..9: note = Synthetic Polypeptide
LQYGSIPFT

SEQ ID NO: 7 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGDATYYRDSVKGRFTISRDNSKNTLYLQMNSLRAEDTAVYYCVRHDYYDGSYGFAYWGQGTLVTVSS

SEQ ID NO: 8 - protein, 107 aa, synthetic construct
  REGION 1..107: note = Synthetic Polypeptide
EIVMTQSPATLSVSPGERVTLSCKASQSVGINVDWYQQKPGQAPRLLIYGASNRHTGIPARFSGSGSGREFTLTISSLQSEDFAVYYCLQYGSIPFTFGPGTKVDIK

SEQ ID NO: 9 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGDATYYRDSVKGRFTISRDNSKNTLYLQMNSLRAEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGREFTLTISSLQSEDFAVYYCLQYGSIPFTFGPGTKVDIK

SEQ ID NO: 10 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGDATYYRDSVKGRFTISRDNSKNTLYLQMNSLRAEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGREFTLTISSLQSEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 11 - protein, 5 aa, synthetic construct
  REGION 1..5: note = Synthetic Polypeptide
NYDMA

SEQ ID NO: 12 - protein, 17 aa, synthetic construct
  REGION 1..17: note = Synthetic Polypeptide
SIITSGDMTYYRDSVKG

SEQ ID NO: 13 - protein, 12 aa, synthetic construct
  REGION 1..12: note = Synthetic Polypeptide
HDYYDGSYGFAY

SEQ ID NO: 14 - protein, 11 aa, synthetic construct
  REGION 1..11: note = Synthetic Polypeptide
KASQSVGINVD

SEQ ID NO: 15 - protein, 7 aa, synthetic construct
  REGION 1..7: note = Synthetic Polypeptide
GASNRHT

SEQ ID NO: 16 - protein, 9 aa, synthetic construct
  REGION 1..9: note = Synthetic Polypeptide
LQYGSIPFT

SEQ ID NO: 17 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGDMTYYRDSVKGRFTISRDNSKNTLYLQMNSLRAEDTAVYYCVRHDYYDGSYGFAYWGQGTLVTVSS

SEQ ID NO: 18 - protein, 107 aa, synthetic construct; identical to SEQ ID NO: 8
  REGION 1..107: note = Synthetic Polypeptide

SEQ ID NO: 19 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGDMTYYRDSVKGRFTISRDNSKNTLYLQMNSLRAEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGREFTLTISSLQSEDFAVYYCLQYGSIPFTFGPGTKVDIK

SEQ ID NO: 20 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGDMTYYRDSVKGRFTISRDNSKNTLYLQMNSLRAEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGREFTLTISSLQSEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 21 - protein, 5 aa, synthetic construct
  REGION 1..5: note = Synthetic Polypeptide
NYDMA

SEQ ID NO: 22 - protein, 17 aa, synthetic construct
  REGION 1..17: note = Synthetic Polypeptide
SIITSGDATYYRDSVKG

SEQ ID NO: 23 - protein, 12 aa, synthetic construct
  REGION 1..12: note = Synthetic Polypeptide
HDYYDGSYGFAY

SEQ ID NO: 24 - protein, 11 aa, synthetic construct
  REGION 1..11: note = Synthetic Polypeptide
KASQSVGINVD

SEQ ID NO: 25 - protein, 7 aa, synthetic construct
  REGION 1..7: note = Synthetic Polypeptide
GASNRHT

SEQ ID NO: 26 - protein, 9 aa, synthetic construct
  REGION 1..9: note = Synthetic Polypeptide
LQYGSIPFT

SEQ ID NO: 27 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGDATYYRDSVKGRFTISRDNSKNTLYLQMNSLRSEDTAVYYCVRHDYYDGSYGFAYWGQGTLVTVSS

SEQ ID NO: 28 - protein, 107 aa, synthetic construct
  REGION 1..107: note = Synthetic Polypeptide
EIVMTQSPATLSVSPGERVTLSCKASQSVGINVDWYQQKPGQAPRLLIYGASNRHTGIPARFSGSGSGTEFTLTISSLQSEDFAVYYCLQYGSIPFTFGPGTKVDIK

SEQ ID NO: 29 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGDATYYRDSVKGRFTISRDNSKNTLYLQMNSLRSEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGTEFTLTISSLQSEDFAVYYCLQYGSIPFTFGPGTKVDIK

SEQ ID NO: 30 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGDATYYRDSVKGRFTISRDNSKNTLYLQMNSLRSEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGTEFTLTISSLQSEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 31 - protein, 5 aa, synthetic construct
  REGION 1..5: note = Synthetic Polypeptide
NYDMA

SEQ ID NO: 32 - protein, 17 aa, synthetic construct
  REGION 1..17: note = Synthetic Polypeptide
SIITSGDMTYYRDSVKG

SEQ ID NO: 33 - protein, 12 aa, synthetic construct
  REGION 1..12: note = Synthetic Polypeptide
HDYYDGSYGFAY

SEQ ID NO: 34 - protein, 11 aa, synthetic construct
  REGION 1..11: note = Synthetic Polypeptide
KASQSVGINVD

SEQ ID NO: 35 - protein, 7 aa, synthetic construct
  REGION 1..7: note = Synthetic Polypeptide
GASNRHT

SEQ ID NO: 36 - protein, 9 aa, synthetic construct
  REGION 1..9: note = Synthetic Polypeptide
LQYGSIPFT

SEQ ID NO: 37 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGDMTYYRDSVKGRFTISRDNSKNTLYLQMNSLRSEDTAVYYCVRHDYYDGSYGFAYWGQGTLVTVSS

SEQ ID NO: 38 - protein, 107 aa, synthetic construct; identical to SEQ ID NO: 28
  REGION 1..107: note = Synthetic Polypeptide

SEQ ID NO: 39 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGDMTYYRDSVKGRFTISRDNSKNTLYLQMNSLRSEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGTEFTLTISSLQSEDFAVYYCLQYGSIPFTFGPGTKVDIK

SEQ ID NO: 40 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGDMTYYRDSVKGRFTISRDNSKNTLYLQMNSLRSEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGTEFTLTISSLQSEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 41 - protein, 5 aa, synthetic construct
  REGION 1..5: note = Synthetic Polypeptide
NYDMA

SEQ ID NO: 42 - protein, 17 aa, synthetic construct
  REGION 1..17: note = Synthetic Polypeptide
SIITSGDATYYRDSVKG

SEQ ID NO: 43 - protein, 12 aa, synthetic construct
  REGION 1..12: note = Synthetic Polypeptide
HDYYDGSYGFAY

SEQ ID NO: 44 - protein, 11 aa, synthetic construct
  REGION 1..11: note = Synthetic Polypeptide
KASQSVGINVD

SEQ ID NO: 45 - protein, 7 aa, synthetic construct
  REGION 1..7: note = Synthetic Polypeptide
GASNRHT

SEQ ID NO: 46 - protein, 9 aa, synthetic construct
  REGION 1..9: note = Synthetic Polypeptide
LQYGSIPFT

SEQ ID NO: 47 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGDATYYRDSVKGRFTISRDNAKNTLYLQMNSLRAEDTAVYYCVRHDYYDGSYGFAYWGQGTLVTVSS

SEQ ID NO: 48 - protein, 107 aa, synthetic construct
  REGION 1..107: note = Synthetic Polypeptide
EIVMTQSPASMSVSPGERATLSCKASQSVGINVDWYQQKPGQAPRLLIYGASNRHTGIPARFSGSGSGTEFTLTISSLQSEDFAVYYCLQYGSIPFTFGPGTKVDIK

SEQ ID NO: 49 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGDATYYRDSVKGRFTISRDNAKNTLYLQMNSLRAEDTAVYYCVRHDYYDGSYGFAYWGQGTLVTVSSGGGGSGGGGSGGGGSEIVMTQSPASMSVSPGERATLSCKASQSVGINVDWYQQKPGQAPRLLIYGASNRHTGIPARFSGSGSGTEFTLTISSLQSEDFAVYYCLQYGSIPFTFGPGTKVDIK

SEQ ID NO: 50 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGDATYYRDSVKGRFTISRDNAKNTLYLQMNSLRAEDTAVYYCVRHDYYDGSYGFAYWGQGTLVTVSSGGGGSGGGGSGGGGSEIVMTQSPASMSVSPGERATLSCKASQSVGINVDWYQQKPGQAPRLLIYGASNRHTGIPARFSGSGSGTEFTLTISSLQSEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 51 - protein, 5 aa, synthetic construct
  REGION 1..5: note = Synthetic Polypeptide
NYDMA

SEQ ID NO: 52 - protein, 17 aa, synthetic construct
  REGION 1..17: note = Synthetic Polypeptide
SIITSGDMTYYRDSVKG

SEQ ID NO: 53 - protein, 12 aa, synthetic construct
  REGION 1..12: note = Synthetic Polypeptide
HDYYDGSYGFAY

SEQ ID NO: 54 - protein, 11 aa, synthetic construct
  REGION 1..11: note = Synthetic Polypeptide
KASQSVGINVD

SEQ ID NO: 55 - protein, 7 aa, synthetic construct
  REGION 1..7: note = Synthetic Polypeptide
GASNRHT

SEQ ID NO: 56 - protein, 9 aa, synthetic construct
  REGION 1..9: note = Synthetic Polypeptide
LQYGSIPFT

SEQ ID NO: 57 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGDMTYYRDSVKGRFTISRDNAKNTLYLQMNSLRAEDTAVYYCVRHDYYDGSYGFAYWGQGTLVTVSS

SEQ ID NO: 58 - protein, 107 aa, synthetic construct; identical to SEQ ID NO: 48
  REGION 1..107: note = Synthetic Polypeptide

SEQ ID NO: 59 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGDMTYYRDSVKGRFTISRDNAKNTLYLQMNSLRAEDTAVYYCVRHDYYDGSYGFAYWGQGTLVTVSSGGGGSGGGGSGGGGSEIVMTQSPASMSVSPGERATLSCKASQSVGINVDWYQQKPGQAPRLLIYGASNRHTGIPARFSGSGSGTEFTLTISSLQSEDFAVYYCLQYGSIPFTFGPGTKVDIK

SEQ ID NO: 60 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGDMTYYRDSVKGRFTISRDNAKNTLYLQMNSLRAEDTAVYYCVRHDYYDGSYGFAYWGQGTLVTVSSGGGGSGGGGSGGGGSEIVMTQSPASMSVSPGERATLSCKASQSVGINVDWYQQKPGQAPRLLIYGASNRHTGIPARFSGSGSGTEFTLTISSLQSEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 61 - protein, 5 aa, synthetic construct
  REGION 1..5: note = Synthetic Polypeptide
NYDMA

SEQ ID NO: 62 - protein, 17 aa, synthetic construct
  REGION 1..17: note = Synthetic Polypeptide
SIITSGDATYYRDSVKG

SEQ ID NO: 63 - protein, 12 aa, synthetic construct
  REGION 1..12: note = Synthetic Polypeptide
HDYYDGSYGFAY

SEQ ID NO: 64 - protein, 11 aa, synthetic construct
  REGION 1..11: note = Synthetic Polypeptide
KASQSVGINVD

SEQ ID NO: 65 - protein, 7 aa, synthetic construct
  REGION 1..7: note = Synthetic Polypeptide
GASNRHT

SEQ ID NO: 66 - protein, 9 aa, synthetic construct
  REGION 1..9: note = Synthetic Polypeptide
LQYGSIPFT

SEQ ID NO: 67 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGDATYYRDSVKGRFTVSRDNSKNTLYLQMNSLRAEDTAVYYCVRHDYYDGSYGFAYWGQGTLVTVSS

SEQ ID NO: 68 - protein, 107 aa, synthetic construct
  REGION 1..107: note = Synthetic Polypeptide
EIVMTQSPATLSVSPGERATLSCKASQSVGINVDWYQQKPGQAPRLLIYGASNRHTGIPARFSGSGSGTEFTLTISSLQAEDFAVYYCLQYGSIPFTFGPGTKVDIK

SEQ ID NO: 69 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGDATYYRDSVKGRFTVSRDNSKNTLYLQMNSLRAEDTAVYYCVRHDYYDGSYGFAYWGQGTLVTVSSGGGGSGGGGSGGGGSEIVMTQSPATLSVSPGERATLSCKASQSVGINVDWYQQKPGQAPRLLIYGASNRHTGIPARFSGSGSGTEFTLTISSLQAEDFAVYYCLQYGSIPFTFGPGTKVDIK

SEQ ID NO: 70 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGDATYYRDSVKGRFTVSRDNSKNTLYLQMNSLRAEDTAVYYCVRHDYYDGSYGFAYWGQGTLVTVSSGGGGSGGGGSGGGGSEIVMTQSPATLSVSPGERATLSCKASQSVGINVDWYQQKPGQAPRLLIYGASNRHTGIPARFSGSGSGTEFTLTISSLQAEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 71 - protein, 5 aa, synthetic construct
  REGION 1..5: note = Synthetic Polypeptide
NYDMA

SEQ ID NO: 72 - protein, 17 aa, synthetic construct
  REGION 1..17: note = Synthetic Polypeptide
SIITSGDMTYYRDSVKG

SEQ ID NO: 73 - protein, 12 aa, synthetic construct
  REGION 1..12: note = Synthetic Polypeptide
HDYYDGSYGFAY

SEQ ID NO: 74 - protein, 11 aa, synthetic construct
  REGION 1..11: note = Synthetic Polypeptide
KASQSVGINVD

SEQ ID NO: 75 - protein, 7 aa, synthetic construct
  REGION 1..7: note = Synthetic Polypeptide
GASNRHT

SEQ ID NO: 76 - protein, 9 aa, synthetic construct
  REGION 1..9: note = Synthetic Polypeptide
LQYGSIPFT

SEQ ID NO: 77 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGDMTYYRDSVKGRFTVSRDNSKNTLYLQMNSLRAEDTAVYYCVRHDYYDGSYGFAYWGQGTLVTVSS

SEQ ID NO: 78 - protein, 107 aa, synthetic construct; identical to SEQ ID NO: 68
  REGION 1..107: note = Synthetic Polypeptide

SEQ ID NO: 79 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGDMTYYRDSVKGRFTVSRDNSKNTLYLQMNSLRAEDTAVYYCVRHDYYDGSYGFAYWGQGTLVTVSSGGGGSGGGGSGGGGSEIVMTQSPATLSVSPGERATLSCKASQSVGINVDWYQQKPGQAPRLLIYGASNRHTGIPARFSGSGSGTEFTLTISSLQAEDFAVYYCLQYGSIPFTFGPGTKVDIK

SEQ ID NO: 80 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGDMTYYRDSVKGRFTVSRDNSKNTLYLQMNSLRAEDTAVYYCVRHDYYDGSYGFAYWGQGTLVTVSSGGGGSGGGGSGGGGSEIVMTQSPATLSVSPGERATLSCKASQSVGINVDWYQQKPGQAPRLLIYGASNRHTGIPARFSGSGSGTEFTLTISSLQAEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 81 - protein, 5 aa, synthetic construct
  REGION 1..5: note = Synthetic Polypeptide
NFDMA

SEQ ID NO: 82 - protein, 17 aa, synthetic construct
  REGION 1..17: note = Synthetic Polypeptide
SITTGADHAIYADSVKG

SEQ ID NO: 83 - protein, 12 aa, synthetic construct
  REGION 1..12: note = Synthetic Polypeptide
HGYYDGYHLFDY

SEQ ID NO: 84 - protein, 11 aa, synthetic construct
  REGION 1..11: note = Synthetic Polypeptide
RASQGISNYLN

SEQ ID NO: 85 - protein, 7 aa, synthetic construct
  REGION 1..7: note = Synthetic Polypeptide
YTSNLQS

SEQ ID NO: 86 - protein, 9 aa, synthetic construct
  REGION 1..9: note = Synthetic Polypeptide
QQYDISSYT

SEQ ID NO: 87 - protein, 121 aa, synthetic construct
  REGION 1..121: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMDSLRAEDTAVYYCVRHGYYDGYHLFDYWGQGTLVTVSS

SEQ ID NO: 88 - protein, 107 aa, synthetic construct
  REGION 1..107: note = Synthetic Polypeptide
DIQMTQSPSSLSASVGDRVTITCRASQGISNYLNWYQQKPGKAPKPLIYYTSNLQSGVPSRFSGSGSGTDYTLTISSLQPEDFATYYCQQYDISSYTFGQGTKLEIK

SEQ ID NO: 89 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCQQYDISSYTFGQGTKLEIK

SEQ ID NO: 90 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCQQYDISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 91 - protein, 5 aa, synthetic construct
  REGION 1..5: note = Synthetic Polypeptide
NFDMA

SEQ ID NO: 92 - protein, 17 aa, synthetic construct
  REGION 1..17: note = Synthetic Polypeptide
SITTGADHAIYADSVKG

SEQ ID NO: 93 - protein, 12 aa, synthetic construct
  REGION 1..12: note = Synthetic Polypeptide
HGYYDGYHLFDY

SEQ ID NO: 94 - protein, 11 aa, synthetic construct
  REGION 1..11: note = Synthetic Polypeptide
RASQGISNYLN

SEQ ID NO: 95 - protein, 7 aa, synthetic construct
  REGION 1..7: note = Synthetic Polypeptide
YTSNLQS

SEQ ID NO: 96 - protein, 9 aa, synthetic construct
  REGION 1..9: note = Synthetic Polypeptide
MGQTISSYT

SEQ ID NO: 97 - protein, 121 aa, synthetic construct; identical to SEQ ID NO: 87
  REGION 1..121: note = Synthetic Polypeptide

SEQ ID NO: 98 - protein, 107 aa, synthetic construct
  REGION 1..107: note = Synthetic Polypeptide
DIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 99 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 100 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 101 - protein, 5 aa, synthetic construct
  REGION 1..5: note = Synthetic Polypeptide
NFDMA

SEQ ID NO: 102 - protein, 17 aa, synthetic construct
  REGION 1..17: note = Synthetic Polypeptide
SITTGADHAIYADSVKG

SEQ ID NO: 103 - protein, 12 aa, synthetic construct
  REGION 1..12: note = Synthetic Polypeptide
HGYYDGYHLFDY

SEQ ID NO: 104 - protein, 11 aa, synthetic construct
  REGION 1..11: note = Synthetic Polypeptide
RASQGISNYLN

SEQ ID NO: 105 - protein, 7 aa, synthetic construct
  REGION 1..7: note = Synthetic Polypeptide
YTSNLQS

SEQ ID NO: 106 - protein, 9 aa, synthetic construct
  REGION 1..9: note = Synthetic Polypeptide
QQYDISSYT

SEQ ID NO: 107 - protein, 121 aa, synthetic construct
  REGION 1..121: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSS

SEQ ID NO: 108 - protein, 107 aa, synthetic construct; identical to SEQ ID NO: 88
  REGION 1..107: note = Synthetic Polypeptide

SEQ ID NO: 109 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCQQYDISSYTFGQGTKLEIK

SEQ ID NO: 110 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCQQYDISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 111 - protein, 5 aa, synthetic construct
  REGION 1..5: note = Synthetic Polypeptide
NFDMA

SEQ ID NO: 112 - protein, 17 aa, synthetic construct
  REGION 1..17: note = Synthetic Polypeptide
SITTGADHAIYADSVKG

SEQ ID NO: 113 - protein, 12 aa, synthetic construct
  REGION 1..12: note = Synthetic Polypeptide
HGYYDGYHLFDY

SEQ ID NO: 114 - protein, 11 aa, synthetic construct
  REGION 1..11: note = Synthetic Polypeptide
RASQGISNYLN

SEQ ID NO: 115 - protein, 7 aa, synthetic construct
  REGION 1..7: note = Synthetic Polypeptide
YTSNLQS

SEQ ID NO: 116 - protein, 9 aa, synthetic construct
  REGION 1..9: note = Synthetic Polypeptide
MGQTISSYT

SEQ ID NO: 117 - protein, 121 aa, synthetic construct; identical to SEQ ID NO: 107
  REGION 1..121: note = Synthetic Polypeptide

SEQ ID NO: 118 - protein, 107 aa, synthetic construct; identical to SEQ ID NO: 98
  REGION 1..107: note = Synthetic Polypeptide

SEQ ID NO: 119 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 120 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 121 - protein, 5 aa, synthetic construct
  REGION 1..5: note = Synthetic Polypeptide
NFDMA

SEQ ID NO: 122 - protein, 17 aa, synthetic construct
  REGION 1..17: note = Synthetic Polypeptide
SITTGADHAIYADSVKG

SEQ ID NO: 123 - protein, 12 aa, synthetic construct
  REGION 1..12: note = Synthetic Polypeptide
HGYYDGYHLFDY

SEQ ID NO: 124 - protein, 11 aa, synthetic construct
  REGION 1..11: note = Synthetic Polypeptide
RASQGISNYLN

SEQ ID NO: 125 - protein, 7 aa, synthetic construct
  REGION 1..7: note = Synthetic Polypeptide
YTSNLQS

SEQ ID NO: 126 - protein, 9 aa, synthetic construct
  REGION 1..9: note = Synthetic Polypeptide
QQYDISSYT

SEQ ID NO: 127 - protein, 121 aa, synthetic construct
  REGION 1..121: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMDSLRSEDTAVYYCVRHGYYDGYHLFDYWGQGTLVTVSS

SEQ ID NO: 128 - protein, 107 aa, synthetic construct
  REGION 1..107: note = Synthetic Polypeptide
DIQMTQSPSSLSASVGDRVTISCRASQGISNYLNWYQQKPGKAPKPLIYYTSNLQSGVPSRFSGSGSGTDYTLTISSLQPEDFATYYCQQYDISSYTFGQGTKLEIK

SEQ ID NO: 129 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMDSLRSEDTAVYYCVRHGYYDGYHLFDYWGQGTLVTVSSGGGGSGGGGSGGGGSDIQMTQSPSSLSASVGDRVTISCRASQGISNYLNWYQQKPGKAPKPLIYYTSNLQSGVPSRFSGSGSGTDYTLTISSLQPEDFATYYCQQYDISSYTFGQGTKLEIK

SEQ ID NO: 130 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGADHAIYADSVKGRFTISRDNAKNTLYLQMDSLRSEDTAVYYCVRHGYYDGYHLFDYWGQGTLVTVSSGGGGSGGGGSGGGGSDIQMTQSPSSLSASVGDRVTISCRASQGISNYLNWYQQKPGKAPKPLIYYTSNLQSGVPSRFSGSGSGTDYTLTISSLQPEDFATYYCQQYDISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 131 - protein, 5 aa, synthetic construct
  REGION 1..5: note = Synthetic Polypeptide
NFDMA

SEQ ID NO: 132 - protein, 17 aa, synthetic construct
  REGION 1..17: note = Synthetic Polypeptide
SITTGADHAIYAESVKG

SEQ ID NO: 133 - protein, 12 aa, synthetic construct
  REGION 1..12: note = Synthetic Polypeptide
HGYYDGYHLFDY

SEQ ID NO: 134 - protein, 11 aa, synthetic construct
  REGION 1..11: note = Synthetic Polypeptide
RASQGISNYLN

SEQ ID NO: 135 - protein, 7 aa, synthetic construct
  REGION 1..7: note = Synthetic Polypeptide
YTSNLQS

SEQ ID NO: 136 - protein, 9 aa, synthetic construct
  REGION 1..9: note = Synthetic Polypeptide
MGQTISSYT

SEQ ID NO: 137 - protein, 121 aa, synthetic construct
  REGION 1..121: note = Synthetic Polypeptide
EVQLVESGGGLVQPGGSLRLSCAASGFTFSNFDMAWVRQAPGKGLVWVSSITTGADHAIYAESVKGRFTISRDNAKNTLYLQMDSLRSEDTAVYYCVRHGYYDGYHLFDYWGQGTLVTVSS

SEQ ID NO: 138 - protein, 107 aa, synthetic construct
  REGION 1..107: note = Synthetic Polypeptide
DIQMTQSPSSLSASVGDRVTISCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 139 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGADHAIYAESVKGRFTISRDNAKNTLYLQMDSLRSEDTAVYYCVRHGYYDGYHLFDYWGQGTLVTVSSGGGGSGGGGSGGGGSDIQMTQSPSSLSASVGDRVTISCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 140 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGADHAIYAESVKGRFTISRDNAKNTLYLQMDSLRSEDTAVYYCVRHGYYDGYHLFDYWGQGTLVTVSSGGGGSGGGGSGGGGSDIQMTQSPSSLSASVGDRVTISCRASQGISNYLNWYQQKPGKAPKPLIYYTSNLQSGVPSRFSGSGSGTDYTLTISSLQPEDFATYYCMGQTISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 141 - protein, 5 aa, synthetic construct
  REGION 1..5: note = Synthetic Polypeptide
NFDMA

SEQ ID NO: 142 - protein, 17 aa, synthetic construct
  REGION 1..17: note = Synthetic Polypeptide
SITTGADHAIYADSVKG

SEQ ID NO: 143 - protein, 12 aa, synthetic construct
  REGION 1..12: note = Synthetic Polypeptide
HGYYDGYHLFDY

SEQ ID NO: 144 - protein, 11 aa, synthetic construct
  REGION 1..11: note = Synthetic Polypeptide
RASQGISNYLN

SEQ ID NO: 145 - protein, 7 aa, synthetic construct
  REGION 1..7: note = Synthetic Polypeptide
YTSNLQS

SEQ ID NO: 146 - protein, 9 aa, synthetic construct
  REGION 1..9: note = Synthetic Polypeptide
MGQTISSYT

SEQ ID NO: 147 - protein, 121 aa, synthetic construct
  REGION 1..121: note = Synthetic Polypeptide
EVQLVESGGGLVQPGRSLRLSCAASGFTFSNFDMAWVRQAPAKGLEWVSSITTGADHAIYADSVKGRFTISRDNAKNTLYLQMNSLRAEDTAVYYCVRHGYYDGYHLFDYWGQGTLVTVSS

SEQ ID NO: 148 - protein, 107 aa, synthetic construct
  REGION 1..107: note = Synthetic Polypeptide
DIQMTQSPSSLSASVGDRVTITCRASQGISNYLNWYQQKPGKAPKPLIYYTSNLQSGVPSRFSGSGSGTDFTLTISSLQPEDFATYYCMGQTISSYTFGQGTKLEIK

SEQ ID NO: 149 - protein, 243 aa, synthetic construct
  REGION 1..243: note = Synthetic Polypeptide
EVQLVESGGGLVQPGRSLRLSCAASGFTFSNFDMAWVRQAPAKGLEWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDFTLTISSLQPEDFATYYCMGQTISSYTFGQGTKLEIK

SEQ ID NO: 150 - protein, 498 aa, synthetic construct
  REGION 1..498: note = Synthetic Polypeptide
EVQLVESGGGLVQPGRSLRLSCAASGFTFSNFDMAWVRQAPAKGLEWVSSITTGADHAIYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDFTLTISSLQPEDFATYYCMGQTISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 151 - protein, 5 aa, synthetic construct
  REGION 1..5: note = Synthetic Polypeptide
NHIIH

SEQ ID NO: 152 - protein, 17 aa, synthetic construct
  REGION 1..17: note = Synthetic Polypeptide
YINPYPGYHAYNEKFQG

SEQ ID NO: 153 - protein, 12 aa, synthetic construct
  REGION 1..12: note = Synthetic Polypeptide
DGYYRDTDVLDY

SEQ ID NO: 154 - protein, 11 aa, synthetic construct
  REGION 1..11: note = Synthetic Polypeptide
QASQDISNYLN

SEQ ID NO: 155 - protein, 7 aa, synthetic construct
  REGION 1..7: note = Synthetic Polypeptide
YTSRLHT

SEQ ID NO: 156 - protein, 9 aa, synthetic construct
  REGION 1..9: note = Synthetic Polypeptide
QQGNTLPWT

SEQ ID NO: 157 - protein, 121 aa, synthetic construct
  REGION 1..121: note = Synthetic Polypeptide
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSS

SEQ ID NO: 158 - protein, 107 aa, synthetic construct
  REGION 1..107: note = Synthetic Polypeptide
DIQMTQSPSSLSASLGDRVTITCQASQDISNYLNWYQQKPGKAPKLLIYYTSRLHTGVPSRFSGSGSGTDFTFTISSLQQEDIATYYCQQGNTLPWTFGQGTKVEIK

SEQ ID NO: 159 - protein, 243 aa, synthetic construct
  REGION 1..243: note = Synthetic Polypeptide
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASLGDRVTITCQASQDISNYLNWYQQKPGKAPKLLIYYTSRLHTGVPSRFSGSGSGTDFTFTISSLQQEDIATYYCQQGNTLPWTFGQGTKVEIK

SEQ ID NO: 160 - protein, 498 aa, synthetic construct
  REGION 1..498: note = Synthetic Polypeptide
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASLGDRVTITCQASQDISNYLNWYQQKPGKAPKLLIYYTSRLHTGVPSRFSGSGSGTDFTFTISSLQQEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 161 - protein, 5 aa, synthetic construct
  REGION 1..5: note = Synthetic Polypeptide
NHIIH

SEQ ID NO: 162 - protein, 17 aa, synthetic construct
  REGION 1..17: note = Synthetic Polypeptide
YINPYDGWGDYNEKFQG

SEQ ID NO: 163 - protein, 12 aa, synthetic construct
  REGION 1..12: note = Synthetic Polypeptide
DGYYRDADVLDY

SEQ ID NO: 164 - protein, 11 aa, synthetic construct
  REGION 1..11: note = Synthetic Polypeptide
QASQDISNYLN

SEQ ID NO: 165 - protein, 7 aa, synthetic construct
  REGION 1..7: note = Synthetic Polypeptide
YTSRLHT

SEQ ID NO: 166 - protein, 9 aa, synthetic construct
  REGION 1..9: note = Synthetic Polypeptide
QQGNTLPWT

SEQ ID NO: 167 - protein, 121 aa, synthetic construct
  REGION 1..121: note = Synthetic Polypeptide
QVQLVQSGAEVKKPGASVKVSCKASGYTFTNHIIHWVRQAPGQGLEWMGYINPYDGWGDYNEKFQGRATMTSDTSTSTVYMELSSLRSEDTAVYYCARDGYYRDADVLDYWGQGTLVTVSS

SEQ ID NO: 168 - protein, 107 aa, synthetic construct; identical to SEQ ID NO: 158
  REGION 1..107: note = Synthetic Polypeptide

SEQ ID NO: 169 - protein, 243 aa, synthetic construct
  REGION 1..243: note = Synthetic Polypeptide
QVQLVQSGAEVKKPGASVKVSCKASGYTFTNHIIHWVRQAPGQGLEWMGYINPYDGWGDYNEKFQGRATMTSDTSTSTVYMELSSLRSEDTAVYYCARDGYYRDADVLDYWGQGTLVTVSSGGGGSGGGGSGGGGSDIQMTQSPSSLSASLGDRVTITCQASQDISNYLNWYQQKPGKAPKLLIYYTSRLHTGVPSRFSGSGSGTDFTFTISSLQQEDIATYYCQQGNTLPWTFGQGTKVEIK

SEQ ID NO: 170 - protein, 498 aa, synthetic construct
  REGION 1..498: note = Synthetic Polypeptide
QVQLVQSGAEVKKPGASVKVSCKASGYTFTNHIIHWVRQAPGQGLEWMGYINPYDGWGDYNEKFQGRATMTSDTSTSTVYMELSSLRSEDTAVYYCARDGYYRDADVLDYWGQGTLVTVSSGGGGSGGGGSGGGGSDIQMTQSPSSLSASLGDRVTITCQASQDISNYLNWYQQKPGKAPKLLIYYTSRLHTGVPSRFSGSGSGTDFTFTISSLQQEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 171 - protein, 5 aa, synthetic construct
  REGION 1..5: note = Synthetic Polypeptide
NHIIH

SEQ ID NO: 172 - protein, 17 aa, synthetic construct
  REGION 1..17: note = Synthetic Polypeptide
YINPYPGYHAYNEKFQG

SEQ ID NO: 173 - protein, 12 aa, synthetic construct
  REGION 1..12: note = Synthetic Polypeptide
DGYYRDTDVLDY

SEQ ID NO: 174 - protein, 11 aa, synthetic construct
  REGION 1..11: note = Synthetic Polypeptide
QASQDISNYLN

SEQ ID NO: 175 - protein, 7 aa, synthetic construct
  REGION 1..7: note = Synthetic Polypeptide
YTSRLHT

SEQ ID NO: 176 - protein, 9 aa, synthetic construct
  REGION 1..9: note = Synthetic Polypeptide
QQGNTLPWT

SEQ ID NO: 177 - protein, 121 aa, synthetic construct; identical to SEQ ID NO: 157
  REGION 1..121: note = Synthetic Polypeptide

SEQ ID NO: 178 - protein, 107 aa, synthetic construct
  REGION 1..107: note = Synthetic Polypeptide
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIK

SEQ ID NO: 179 - protein, 243 aa, synthetic construct
  REGION 1..243: note = Synthetic Polypeptide
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIK

SEQ ID NO: 180 - protein, 498 aa, synthetic construct
  REGION 1..498: note = Synthetic Polypeptide
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 181 - protein, 5 aa, synthetic construct
  REGION 1..5: note = Synthetic Polypeptide
NHIIH

SEQ ID NO: 182 - protein, 17 aa, synthetic construct
  REGION 1..17: note = Synthetic Polypeptide
YINPYDGWGDYNEKFQG

SEQ ID NO: 183 - protein, 12 aa, synthetic construct
  REGION 1..12: note = Synthetic Polypeptide
DGYYRDADVLDY

SEQ ID NO: 184 - protein, 11 aa, synthetic construct
  REGION 1..11: note = Synthetic Polypeptide
QASQDISNYLN

SEQ ID NO: 185 - protein, 7 aa, synthetic construct
  REGION 1..7: note = Synthetic Polypeptide
YTSRLHT

SEQ ID NO: 186 - protein, 9 aa, synthetic construct
  REGION 1..9: note = Synthetic Polypeptide
QQGNTLPWT

SEQ ID NO: 187 - protein, 121 aa, synthetic construct; identical to SEQ ID NO: 167
  REGION 1..121: note = Synthetic Polypeptide

SEQ ID NO: 188 - protein, 107 aa, synthetic construct; identical to SEQ ID NO: 178
  REGION 1..107: note = Synthetic Polypeptide

SEQ ID NO: 189 - protein, 243 aa, synthetic construct
  REGION 1..243: note = Synthetic Polypeptide
QVQLVQSGAEVKKPGASVKVSCKASGYTFTNHIIHWVRQAPGQGLEWMGYINPYDGWGDYNEKFQGRATMTSDTSTSTVYMELSSLRSEDTAVYYCARDGYYRDADVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIK

SEQ ID NO: 190 - protein, 498 aa, synthetic construct
  REGION 1..498: note = Synthetic Polypeptide
QVQLVQSGAEVKKPGASVKVSCKASGYTFTNHIIHWVRQAPGQGLEWMGYINPYDGWGDYNEKFQGRATMTSDTSTSTVYMELSSLRSEDTAVYYCARDGYYRDADVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 191 - protein, 5 aa, synthetic construct
  REGION 1..5: note = Synthetic Polypeptide
NHIIH

SEQ ID NO: 192 - protein, 17 aa, synthetic construct
  REGION 1..17: note = Synthetic Polypeptide
YINPYPGYHAYNQKFQG

SEQ ID NO: 193 - protein, 12 aa, synthetic construct
  REGION 1..12: note = Synthetic Polypeptide
DGYYRDTDVLDY

SEQ ID NO: 194 - protein, 11 aa, synthetic construct
  REGION 1..11: note = Synthetic Polypeptide
QASQDISNYLN

SEQ ID NO: 195 - protein, 7 aa, synthetic construct
  REGION 1..7: note = Synthetic Polypeptide
YTSRLHT

SEQ ID NO: 196 - protein, 9 aa, synthetic construct
  REGION 1..9: note = Synthetic Polypeptide
QQGNTLPWT

SEQ ID NO: 197 - protein, 121 aa, synthetic construct
  REGION 1..121: note = Synthetic Polypeptide
QVQLVQSGAKVIKPGASVKVSCKASGYTFTNHIIHWVRQKPGQGLEWMGYINPYPGYHAYNQKFQGRVTMTRDKSTSTVYMELSSLTSEDTAVYYCARDGYYRDTDVLDYWGQGTLVTVSS

SEQ ID NO: 198 - protein, 107 aa, synthetic construct
  REGION 1..107: note = Synthetic Polypeptide
DIQMTQSPSSLSASVGDRVTITCQASQDISNYLNWYQQKPGRAPKLLIYYTSRLHTGVPSRFSGSGSGTDYSFTISSLQPEDIATYYCQQGNTLPWTFGQGTKVEIK

SEQ ID NO: 199 - protein, 243 aa, synthetic construct
  REGION 1..243: note = Synthetic Polypeptide
QVQLVQSGAKVIKPGASVKVSCKASGYTFTNHIIHWVRQKPGQGLEWMGYINPYPGYHAYNQKFQGRVTMTRDKSTSTVYMELSSLTSEDTAVYYCARDGYYRDTDVLDYWGQGTLVTVSSGGGGSGGGGSGGGGSDIQMTQSPSSLSASVGDRVTITCQASQDISNYLNWYQQKPGRAPKLLIYYTSRLHTGVPSRFSGSGSGTDYSFTISSLQPEDIATYYCQQGNTLPWTFGQGTKVEIK

SEQ ID NO: 200 - protein, 498 aa, synthetic construct
  REGION 1..498: note = Synthetic Polypeptide
QVQLVQSGAKVIKPGASVKVSCKASGYTFTNHIIHWVRQKPGQGLEWMGYINPYPGYHAYNQKFQGRVTMTRDKSTSTVYMELSSLTSEDTAVYYCARDGYYRDTDVLDYWGQGTLVTVSSGGGGSGGGGSGGGGSDIQMTQSPSSLSASVGDRVTITCQASQDISNYLNWYQQKPGRAPKLLIYYTSRLHTGVPSRFSGSGSGTDYSFTISSLQPEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 201 - protein, 5 aa, synthetic construct
  REGION 1..5: note = Synthetic Polypeptide
NHIIH

SEQ ID NO: 202 - protein, 17 aa, synthetic construct
  REGION 1..17: note = Synthetic Polypeptide
YINPYDGWGDYNQKFQG

SEQ ID NO: 203 - protein, 12 aa, synthetic construct
  REGION 1..12: note = Synthetic Polypeptide
DGYYRDADVLDY

SEQ ID NO: 204 - protein, 11 aa, synthetic construct
  REGION 1..11: note = Synthetic Polypeptide
QASQDISNYLN

SEQ ID NO: 205 - protein, 7 aa, synthetic construct
  REGION 1..7: note = Synthetic Polypeptide
YTSRLHT

SEQ ID NO: 206 - protein, 9 aa, synthetic construct
  REGION 1..9: note = Synthetic Polypeptide
QQGNTLPWT

SEQ ID NO: 207 - protein, 121 aa, synthetic construct
  REGION 1..121: note = Synthetic Polypeptide
QVQLVQSGAKVIKPGASVKVSCKASGYTFTNHIIHWVRQKPGQGLEWMGYINPYDGWGDYNQKFQGRVTMTRDKSTSTVYMELSSLTSEDTAVYYCARDGYYRDADVLDYWGQGTLVTVSS

SEQ ID NO: 208 - protein, 107 aa, synthetic construct; identical to SEQ ID NO: 198
  REGION 1..107: note = Synthetic Polypeptide

SEQ ID NO: 209 - protein, 243 aa, synthetic construct
  REGION 1..243: note = Synthetic Polypeptide
QVQLVQSGAKVIKPGASVKVSCKASGYTFTNHIIHWVRQKPGQGLEWMGYINPYDGWGDYNQKFQGRVTMTRDKSTSTVYMELSSLTSEDTAVYYCARDGYYRDADVLDYWGQGTLVTVSSGGGGSGGGGSGGGGSDIQMTQSPSSLSASVGDRVTITCQASQDISNYLNWYQQKPGRAPKLLIYYTSRLHTGVPSRFSGSGSGTDYSFTISSLQPEDIATYYCQQGNTLPWTFGQGTKVEIK

SEQ ID NO: 210 - protein, 498 aa, synthetic construct
  REGION 1..498: note = Synthetic Polypeptide
QVQLVQSGAKVIKPGASVKVSCKASGYTFTNHIIHWVRQKPGQGLEWMGYINPYDGWGDYNQKFQGRVTMTRDKSTSTVYMELSSLTSEDTAVYYCARDGYYRDADVLDYWGQGTLVTVSSGGGGSGGGGSGGGGSDIQMTQSPSSLSASVGDRVTITCQASQDISNYLNWYQQKPGRAPKLLIYYTSRLHTGVPSRFSGSGSGTDYSFTISSLQPEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 211 - protein, 5 aa, synthetic construct
  REGION 1..5: note = Synthetic Polypeptide
NHIIH

SEQ ID NO: 212 - protein, 17 aa, synthetic construct
  REGION 1..17: note = Synthetic Polypeptide
YINPYPGYHAYNQKFQG

SEQ ID NO: 213 - protein, 12 aa, synthetic construct
  REGION 1..12: note = Synthetic Polypeptide
DGYYRDTDVLDY

SEQ ID NO: 214 - protein, 11 aa, synthetic construct
  REGION 1..11: note = Synthetic Polypeptide
QASQDISNYLN

SEQ ID NO: 215 - protein, 7 aa, synthetic construct
  REGION 1..7: note = Synthetic Polypeptide
YTSRLHT

SEQ ID NO: 216 - protein, 9 aa, synthetic construct
  REGION 1..9: note = Synthetic Polypeptide
QQGNTLPWT

SEQ ID NO: 217 - protein, 121 aa, synthetic construct
  REGION 1..121: note = Synthetic Polypeptide
QVQLVQSGAEVIKPGASVKVSCKASGYTFTNHIIHWVRQKPGQGLEWIGYINPYPGYHAYNQKFQGKVTMTRDTSTSTVYMELSSLTSEDTAVYYCARDGYYRDTDVLDYWGQGTLVTVSS

SEQ ID NO: 218 - protein, 107 aa, synthetic construct; identical to SEQ ID NO: 158
  REGION 1..107: note = Synthetic Polypeptide

SEQ ID NO: 219 - protein, 243 aa, synthetic construct
  REGION 1..243: note = Synthetic Polypeptide
QVQLVQSGAEVIKPGASVKVSCKASGYTFTNHIIHWVRQKPGQGLEWIGYINPYPGYHAYNQKFQGKVTMTRDTSTSTVYMELSSLTSEDTAVYYCARDGYYRDTDVLDYWGQGTLVTVSSGGGGSGGGGSGGGGSDIQMTQSPSSLSASLGDRVTITCQASQDISNYLNWYQQKPGKAPKLLIYYTSRLHTGVPSRFSGSGSGTDFTFTISSLQQEDIATYYCQQGNTLPWTFGQGTKVEIK

SEQ ID NO: 220 - protein, 498 aa, synthetic construct
  REGION 1..498: note = Synthetic Polypeptide
QVQLVQSGAEVIKPGASVKVSCKASGYTFTNHIIHWVRQKPGQGLEWIGYINPYPGYHAYNQKFQGKVTMTRDTSTSTVYMELSSLTSEDTAVYYCARDGYYRDTDVLDYWGQGTLVTVSSGGGGSGGGGSGGGGSDIQMTQSPSSLSASLGDRVTITCQASQDISNYLNWYQQKPGKAPKLLIYYTSRLHTGVPSRFSGSGSGTDFTFTISSLQQEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 221 - protein, 5 aa, synthetic construct
  REGION 1..5: note = Synthetic Polypeptide
NHIIH

SEQ ID NO: 222 - protein, 17 aa, synthetic construct
  REGION 1..17: note = Synthetic Polypeptide
YINPYDGWGDYNQKFQG

SEQ ID NO: 223 - protein, 12 aa, synthetic construct
  REGION 1..12: note = Synthetic Polypeptide
DGYYRDADVLDY

SEQ ID NO: 224 - protein, 11 aa, synthetic construct
  REGION 1..11: note = Synthetic Polypeptide
QASQDISNYLN

SEQ ID NO: 225 - protein, 7 aa, synthetic construct
  REGION 1..7: note = Synthetic Polypeptide
YTSRLHT

SEQ ID NO: 226 - protein, 9 aa, synthetic construct
  REGION 1..9: note = Synthetic Polypeptide
QQGNTLPWT

SEQ ID NO: 227 - protein, 121 aa, synthetic construct
  REGION 1..121: note = Synthetic Polypeptide
QVQLVQSGAEVIKPGASVKVSCKASGYTFTNHIIHWVRQKPGQGLEWIGYINPYDGWGDYNQKFQGKVTMTRDTSTSTVYMELSSLTSEDTAVYYCARDGYYRDADVLDYWGQGTLVTVSS

SEQ ID NO: 228 - protein, 107 aa, synthetic construct; identical to SEQ ID NO: 158
  REGION 1..107: note = Synthetic Polypeptide

SEQ ID NO: 229 - protein, 243 aa, synthetic construct
  REGION 1..243: note = Synthetic Polypeptide
QVQLVQSGAEVIKPGASVKVSCKASGYTFTNHIIHWVRQKPGQGLEWIGYINPYDGWGDYNQKFQGKVTMTRDTSTSTVYMELSSLTSEDTAVYYCARDGYYRDADVLDYWGQGTLVTVSSGGGGSGGGGSGGGGSDIQMTQSPSSLSASLGDRVTITCQASQDISNYLNWYQQKPGKAPKLLIYYTSRLHTGVPSRFSGSGSGTDFTFTISSLQQEDIATYYCQQGNTLPWTFGQGTKVEIK

SEQ ID NO: 230 - protein, 498 aa, synthetic construct
  REGION 1..498: note = Synthetic Polypeptide
QVQLVQSGAEVIKPGASVKVSCKASGYTFTNHIIHWVRQKPGQGLEWIGYINPYDGWGDYNQKFQGKVTMTRDTSTSTVYMELSSLTSEDTAVYYCARDGYYRDADVLDYWGQGTLVTVSSGGGGSGGGGSGGGGSDIQMTQSPSSLSASLGDRVTITCQASQDISNYLNWYQQKPGKAPKLLIYYTSRLHTGVPSRFSGSGSGTDFTFTISSLQQEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 231 - protein, 5 aa, synthetic construct
  REGION 1..5: note = Synthetic Polypeptide
NYDMA

SEQ ID NO: 232 - protein, 17 aa, synthetic construct
  REGION 1..17: note = Synthetic Polypeptide
SIITSGGDNYYRDSVKG

SEQ ID NO: 233 - protein, 12 aa, synthetic construct
  REGION 1..12: note = Synthetic Polypeptide
HDYYDGSYGFAY

SEQ ID NO: 234 - protein, 11 aa, synthetic construct
  REGION 1..11: note = Synthetic Polypeptide
KASQSVGINVD

SEQ ID NO: 235 - protein, 7 aa, synthetic construct
  REGION 1..7: note = Synthetic Polypeptide
GASNRHT

SEQ ID NO: 236 - protein, 9 aa, synthetic construct
  REGION 1..9: note = Synthetic Polypeptide
LQYGSIPFT

SEQ ID NO: 237 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGGDNYYRDSVKGRFTISRDNAKNTLYLQMNSLRAEDTAVYYCVRHDYYDGSYGFAYWGQGTLVTVSS

SEQ ID NO: 238 - protein, 107 aa, synthetic construct; identical to SEQ ID NO: 48
  REGION 1..107: note = Synthetic Polypeptide

SEQ ID NO: 239 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGGDNYYRDSVKGRFTISRDNAKNTLYLQMNSLRAEDTAVYYCVRHDYYDGSYGFAYWGQGTLVTVSSGGGGSGGGGSGGGGSEIVMTQSPASMSVSPGERATLSCKASQSVGINVDWYQQKPGQAPRLLIYGASNRHTGIPARFSGSGSGTEFTLTISSLQSEDFAVYYCLQYGSIPFTFGPGTKVDIK

SEQ ID NO: 240 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGGDNYYRDSVKGRFTISRDNAKNTLYLQMNSLRAEDTAVYYCVRHDYYDGSYGFAYWGQGTLVTVSSGGGGSGGGGSGGGGSEIVMTQSPASMSVSPGERATLSCKASQSVGINVDWYQQKPGQAPRLLIYGASNRHTGIPARFSGSGSGTEFTLTISSLQSEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 241 - protein, 5 aa, synthetic construct
  REGION 1..5: note = Synthetic Polypeptide
NYDMA

SEQ ID NO: 242 - protein, 17 aa, synthetic construct
  REGION 1..17: note = Synthetic Polypeptide
SIITSGGDNYYRDSVKG

SEQ ID NO: 243 - protein, 12 aa, synthetic construct
  REGION 1..12: note = Synthetic Polypeptide
HDYYDGSYGFAY

SEQ ID NO: 244 - protein, 11 aa, synthetic construct
  REGION 1..11: note = Synthetic Polypeptide
KASQSVGINVD

SEQ ID NO: 245 - protein, 7 aa, synthetic construct
  REGION 1..7: note = Synthetic Polypeptide
GASNRHT

SEQ ID NO: 246 - protein, 9 aa, synthetic construct
  REGION 1..9: note = Synthetic Polypeptide
LQYGSIPFT

SEQ ID NO: 247 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGGDNYYRDSVKGRFTISRDNSKNTLYLQMNSLRAEDTAVYYCVRHDYYDGSYGFAYWGQGTLVTVSS

SEQ ID NO: 248 - protein, 107 aa, synthetic construct; identical to SEQ ID NO: 8
  REGION 1..107: note = Synthetic Polypeptide

SEQ ID NO: 249 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGGDNYYRDSVKGRFTISRDNSKNTLYLQMNSLRAEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGREFTLTISSLQSEDFAVYYCLQYGSIPFTFGPGTKVDIK

SEQ ID NO: 250 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGGDNYYRDSVKGRFTISRDNSKNTLYLQMNSLRAEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGREFTLTISSLQSEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 251 - protein, 5 aa, synthetic construct
  REGION 1..5: note = Synthetic Polypeptide
NYDMA

SEQ ID NO: 252 - protein, 17 aa, synthetic construct
  REGION 1..17: note = Synthetic Polypeptide
SIITSGGDNYYRDSVKG

SEQ ID NO: 253 - protein, 12 aa, synthetic construct
  REGION 1..12: note = Synthetic Polypeptide
HDYYDGSYGFAY

SEQ ID NO: 254 - protein, 11 aa, synthetic construct
  REGION 1..11: note = Synthetic Polypeptide
KASQSVGINVD

SEQ ID NO: 255 - protein, 7 aa, synthetic construct
  REGION 1..7: note = Synthetic Polypeptide
GASNRHT

SEQ ID NO: 256 - protein, 9 aa, synthetic construct
  REGION 1..9: note = Synthetic Polypeptide
LQYGSIPFT

SEQ ID NO: 257 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGGDNYYRDSVKGRFTISRDNSKNTLYLQMNSLRSEDTAVYYCVRHDYYDGSYGFAYWGQGTLVTVSS

SEQ ID NO: 258 - protein, 107 aa, synthetic construct; identical to SEQ ID NO: 28
  REGION 1..107: note = Synthetic Polypeptide

SEQ ID NO: 259 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGGDNYYRDSVKGRFTISRDNSKNTLYLQMNSLRSEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGTEFTLTISSLQSEDFAVYYCLQYGSIPFTFGPGTKVDIK

SEQ ID NO: 260 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGGDNYYRDSVKGRFTISRDNSKNTLYLQMNSLRSEDTAVYYCVRHDYYDGSYGFAYWGQGTLVTVSSGGGGSGGGGSGGGGSEIVMTQSPATLSVSPGERVTLSCKASQSVGINVDWYQQKPGQAPRLLIYGASNRHTGIPARFSGSGSGTEFTLTISSLQSEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 261 - protein, 5 aa, synthetic construct
  REGION 1..5: note = Synthetic Polypeptide
NYDMA

SEQ ID NO: 262 - protein, 17 aa, synthetic construct
  REGION 1..17: note = Synthetic Polypeptide
SIITSGGDNYYRDSVKG

SEQ ID NO: 263 - protein, 12 aa, synthetic construct
  REGION 1..12: note = Synthetic Polypeptide
HDYYDGSYGFAY

SEQ ID NO: 264 - protein, 11 aa, synthetic construct
  REGION 1..11: note = Synthetic Polypeptide
KASQSVGINVD

SEQ ID NO: 265 - protein, 7 aa, synthetic construct
  REGION 1..7: note = Synthetic Polypeptide
GASNRHT

SEQ ID NO: 266 - protein, 9 aa, synthetic construct
  REGION 1..9: note = Synthetic Polypeptide
LQYGSIPFT

SEQ ID NO: 267 - protein, 121 aa, synthetic construct
  REGION 1..121: note = Synthetic Polypeptide
QVQLVESGGGVVQPGRSLRLSCAASGFTFSNYDMAWVRQAPGKGLEWVASIITSGGDNYYRDSVKGRFTVSRDNSKNTLYLQMNSLRAEDTAVYYCVRHDYYDGSYGFAYWGQGTLVTVSS

SEQ ID NO: 268 - protein, 107 aa, synthetic construct; identical to SEQ ID NO: 68
  REGION 1..107: note = Synthetic Polypeptide

SEQ ID NO: 269 - protein, 243 aa, synthetic construct
  REGION 1..243: note = Synthetic Polypeptide
QVQLVESGGGVVQPGRSLRLSCAASGFTFSNYDMAWVRQAPGKGLEWVASIITSGGDNYYRDSVKGRFTVSRDNSKNTLYLQMNSLRAEDTAVYYCVRHDYYDGSYGFAYWGQGTLVTVSSGGGGSGGGGSGGGGSEIVMTQSPATLSVSPGERATLSCKASQSVGINVDWYQQKPGQAPRLLIYGASNRHTGIPARFSGSGSGTEFTLTISSLQAEDFAVYYCLQYGSIPFTFGPGTKVDIK

SEQ ID NO: 270 - protein, 498 aa, synthetic construct
  REGION 1..498: note = Synthetic Polypeptide
QVQLVESGGGVVQPGRSLRLSCAASGFTFSNYDMAWVRQAPGKGLEWVASIITSGGDNYYRDSVKGRFTVSRDNSKNTLYLQMNSLRAEDTAVYYCVRHDYYDGSYGFAYWGQGTLVTVSSGGGGSGGGGSGGGGSEIVMTQSPATLSVSPGERATLSCKASQSVGINVDWYQQKPGQAPRLLIYGASNRHTGIPARFSGSGSGTEFTLTISSLQAEDFAVYYCLQYGSIPFTFGPGTKVDIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 271 - protein, 5 aa, synthetic construct
  REGION 1..5: note = Synthetic Polypeptide
NYDMA

SEQ ID NO: 272 - protein, 17 aa, synthetic construct
  REGION 1..17: note = Synthetic Polypeptide
SISTRGDITSYRDSVKG

SEQ ID NO: 273 - protein, 12 aa, synthetic construct
  REGION 1..12: note = Synthetic Polypeptide
QDYYTDYMGFAY

SEQ ID NO: 274 - protein, 11 aa, synthetic construct
  REGION 1..11: note = Synthetic Polypeptide
RASEDIYNGLA

SEQ ID NO: 275 - protein, 7 aa, synthetic construct
  REGION 1..7: note = Synthetic Polypeptide
GASSLQD

SEQ ID NO: 276 - protein, 9 aa, synthetic construct
  REGION 1..9: note = Synthetic Polypeptide
QQSYKYPLT

SEQ ID NO: 277 - protein, 121 aa, synthetic construct
  REGION 1..121: note = Synthetic Polypeptide
EVQLLESGGGLVQPGGSLRLSCAASGFTFSNYDMAWVRQAPGKGLEWVSSISTRGDITSYRDSVKGRFTISRDNAKNTLYLQMNSLRAEDTAVYYCARQDYYTDYMGFAYWGQGTLVTVSS

SEQ ID NO: 278 - protein, 107 aa, synthetic construct
  REGION 1..107: note = Synthetic Polypeptide
AIQMTQSPSSLSASVGDTVTITCRASEDIYNGLAWYQQKPGKAPKLLIYGASSLQDGVPSRFSGSGSGTDYTLTISSLQPEDEATYYCQQSYKYPLTFGGGTKVEIK

SEQ ID NO: 279 - protein, 243 aa, synthetic construct
  REGION 1..243: note = Synthetic Polypeptide
EVQLLESGGGLVQPGGSLRLSCAASGFTFSNYDMAWVRQAPGKGLEWVSSISTRGDITSYRDSVKGRFTISRDNAKNTLYLQMNSLRAEDTAVYYCARQDYYTDYMGFAYWGQGTLVTVSSGGGGSGGGGSGGGGSAIQMTQSPSSLSASVGDTVTITCRASEDIYNGLAWYQQKPGKAPKLLIYGASSLQDGVPSRFSGSGSGTDYTLTISSLQPEDEATYYCQQSYKYPLTFGGGTKVEIK

SEQ ID NO: 280 - protein, 498 aa, synthetic construct
  REGION 1..498: note = Synthetic Polypeptide
EVQLLESGGGLVQPGGSLRLSCAASGFTFSNYDMAWVRQAPGKGLEWVSSISTRGDITSYRDSVKGRFTISRDNAKNTLYLQMNSLRAEDTAVYYCARQDYYTDYMGFAYWGQGTLVTVSSGGGGSGGGGSGGGGSAIQMTQSPSSLSASVGDTVTITCRASEDIYNGLAWYQQKPGKAPKLLIYGASSLQDGVPSRFSGSGSGTDYTLTISSLQPEDEATYYCQQSYKYPLTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 281 - protein, 5 aa, synthetic construct
  REGION 1..5: note = Synthetic Polypeptide
NYDMA

SEQ ID NO: 282 - protein, 17 aa, synthetic construct
  REGION 1..17: note = Synthetic Polypeptide
SISTRGDITSYRDSVKG

SEQ ID NO: 283 - protein, 12 aa, synthetic construct
  REGION 1..12: note = Synthetic Polypeptide
QDYYTDYMGFAY

SEQ ID NO: 284 - protein, 11 aa, synthetic construct
  REGION 1..11: note = Synthetic Polypeptide
RASEDIYNGLA

SEQ ID NO: 285 - protein, 7 aa, synthetic construct
  REGION 1..7: note = Synthetic Polypeptide
GASSLQD

SEQ ID NO: 286 - protein, 9 aa, synthetic construct
  REGION 1..9: note = Synthetic Polypeptide
QQSYKYPLT

SEQ ID NO: 287 - protein, 121 aa, synthetic construct
  REGION 1..121: note = Synthetic Polypeptide
EVQLLESGGGLVQPGRSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSS

SEQ ID NO: 288 - protein, 107 aa, synthetic construct
  REGION 1..107: note = Synthetic Polypeptide
AIQMTQSPSSLSASVGDRVTITCRASEDIYNGLAWYQQKPGKAPKLLIYGASSLQDGVPSRFSGSGSGTDFTLTISSMQPEDEATYYCQQSYKYPLTFGGGTKVEIK

SEQ ID NO: 289 - protein, 243 aa, synthetic construct
  REGION 1..243: note = Synthetic Polypeptide
EVQLLESGGGLVQPGRSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTDFTLTISSMQPEDEATYYCQQSYKYPLTFGGGTKVEIK

SEQ ID NO: 290 - protein, 498 aa, synthetic construct
  REGION 1..498: note = Synthetic Polypeptide
EVQLLESGGGLVQPGRSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTDFTLTISSMQPEDEATYYCQQSYKYPLTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 291 - protein, 5 aa, synthetic construct
  REGION 1..5: note = Synthetic Polypeptide
NYDMA

SEQ ID NO: 292 - protein, 17 aa, synthetic construct
  REGION 1..17: note = Synthetic Polypeptide
SISTRGDITSYRDSVKG

SEQ ID NO: 293 - protein, 12 aa, synthetic construct
  REGION 1..12: note = Synthetic Polypeptide
QDYYTDYMGFAY

SEQ ID NO: 294 - protein, 11 aa, synthetic construct
  REGION 1..11: note = Synthetic Polypeptide
RASEDIYNGLA

SEQ ID NO: 295 - protein, 7 aa, synthetic construct
  REGION 1..7: note = Synthetic Polypeptide
GASSLQD

SEQ ID NO: 296 - protein, 9 aa, synthetic construct
  REGION 1..9: note = Synthetic Polypeptide
QQSYKYPLT

SEQ ID NO: 297 - protein, 121 aa, synthetic construct
  REGION 1..121: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSS

SEQ ID NO: 298 - protein, 107 aa, synthetic construct
  REGION 1..107: note = Synthetic Polypeptide
AIQMTQSPSSLSASVGDRVTITCRASEDIYNGLAWYQQKPGKAPKLLIYGASSLQDGVPSRFSGSGSGTHYTLTISSLQPEDEATYYCQQSYKYPLTFGGGTKVEIK

SEQ ID NO: 299 - protein, 243 aa, synthetic construct
  REGION 1..243: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTHYTLTISSLQPEDEATYYCQQSYKYPLTFGGGTKVEIK

SEQ ID NO: 300 - protein, 498 aa, synthetic construct
  REGION 1..498: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTHYTLTISSLQPEDEATYYCQQSYKYPLTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 301 - protein, 5 aa, synthetic construct
  REGION 1..5: note = Synthetic Polypeptide
NYDMA

SEQ ID NO: 302 - protein, 17 aa, synthetic construct
  REGION 1..17: note = Synthetic Polypeptide
SISTRGDITSYRDSVKG

SEQ ID NO: 303 - protein, 12 aa, synthetic construct
  REGION 1..12: note = Synthetic Polypeptide
QDYYTDYMGFAY

SEQ ID NO: 304 - protein, 11 aa, synthetic construct
  REGION 1..11: note = Synthetic Polypeptide
RASEDIYNGLA

SEQ ID NO: 305 - protein, 7 aa, synthetic construct
  REGION 1..7: note = Synthetic Polypeptide
GASSLQD

SEQ ID NO: 306 - protein, 9 aa, synthetic construct
  REGION 1..9: note = Synthetic Polypeptide
AGPHKYPLT

SEQ ID NO: 307 - protein, 121 aa, synthetic construct; identical to SEQ ID NO: 277
  REGION 1..121: note = Synthetic Polypeptide

SEQ ID NO: 308 - protein, 107 aa, synthetic construct
  REGION 1..107: note = Synthetic Polypeptide
AIQMTQSPSSLSASVGDTVTITCRASEDIYNGLAWYQQKPGKAPKLLIYGASSLQDGVPSRFSGSGSGTDYTLTISSLQPEDEATYYCAGPHKYPLTFGGGTKVEIK

SEQ ID NO: 309 - protein, 243 aa, synthetic construct
  REGION 1..243: note = Synthetic Polypeptide
EVQLLESGGGLVQPGGSLRLSCAASGFTFSNYDMAWVRQAPGKGLEWVSSISTRGDITSYRDSVKGRFTISRDNAKNTLYLQMNSLRAEDTAVYYCARQDYYTDYMGFAYWGQGTLVTVSSGGGGSGGGGSGGGGSAIQMTQSPSSLSASVGDTVTITCRASEDIYNGLAWYQQKPGKAPKLLIYGASSLQDGVPSRFSGSGSGTDYTLTISSLQPEDEATYYCAGPHKYPLTFGGGTKVEIK

SEQ ID NO: 310 - protein, 498 aa, synthetic construct
  REGION 1..498: note = Synthetic Polypeptide
EVQLLESGGGLVQPGGSLRLSCAASGFTFSNYDMAWVRQAPGKGLEWVSSISTRGDITSYRDSVKGRFTISRDNAKNTLYLQMNSLRAEDTAVYYCARQDYYTDYMGFAYWGQGTLVTVSSGGGGSGGGGSGGGGSAIQMTQSPSSLSASVGDTVTITCRASEDIYNGLAWYQQKPGKAPKLLIYGASSLQDGVPSRFSGSGSGTDYTLTISSLQPEDEATYYCAGPHKYPLTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 311 - protein, 5 aa, synthetic construct
  REGION 1..5: note = Synthetic Polypeptide
NYDMA

SEQ ID NO: 312 - protein, 17 aa, synthetic construct
  REGION 1..17: note = Synthetic Polypeptide
SISTRGDITSYRDSVKG

SEQ ID NO: 313 - protein, 12 aa, synthetic construct
  REGION 1..12: note = Synthetic Polypeptide
QDYYTDYMGFAY

SEQ ID NO: 314 - protein, 11 aa, synthetic construct
  REGION 1..11: note = Synthetic Polypeptide
RASEDIYNGLA

SEQ ID NO: 315 - protein, 7 aa, synthetic construct
  REGION 1..7: note = Synthetic Polypeptide
GASSLQD

SEQ ID NO: 316 - protein, 9 aa, synthetic construct
  REGION 1..9: note = Synthetic Polypeptide
AGPHKYPLT

SEQ ID NO: 317 - protein, 121 aa, synthetic construct; identical to SEQ ID NO: 297
  REGION 1..121: note = Synthetic Polypeptide

SEQ ID NO: 318 - protein, 107 aa, synthetic construct
  REGION 1..107: note = Synthetic Polypeptide
AIQMTQSPSSLSASVGDRVTITCRASEDIYNGLAWYQQKPGKAPKLLIYGASSLQDGVPSRFSGSGSGTHYTLTISSLQPEDEATYYCAGPHKYPLTFGGGTKVEIK

SEQ ID NO: 319 - protein, 243 aa, synthetic construct
  REGION 1..243: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTHYTLTISSLQPEDEATYYCAGPHKYPLTFGGGTKVEIK

SEQ ID NO: 320 - protein, 498 aa, synthetic construct
  REGION 1..498: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTHYTLTISSLQPEDEATYYCAGPHKYPLTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 321 - protein, 5 aa, synthetic construct
  REGION 1..5: note = Synthetic Polypeptide
NYDMA

SEQ ID NO: 322 - protein, 17 aa, synthetic construct
  REGION 1..17: note = Synthetic Polypeptide
SISTRGDITSYRDSVKG

SEQ ID NO: 323 - protein, 12 aa, synthetic construct
  REGION 1..12: note = Synthetic Polypeptide
QDYYTDYMGFAY

SEQ ID NO: 324 - protein, 11 aa, synthetic construct
  REGION 1..11: note = Synthetic Polypeptide
RASEDIYNGLA

SEQ ID NO: 325 - protein, 7 aa, synthetic construct
  REGION 1..7: note = Synthetic Polypeptide
GASSLQD

SEQ ID NO: 326 - protein, 9 aa, synthetic construct
  REGION 1..9: note = Synthetic Polypeptide
QQSRNYQQT

SEQ ID NO: 327 - protein, 121 aa, synthetic construct; identical to SEQ ID NO: 277
  REGION 1..121: note = Synthetic Polypeptide

SEQ ID NO: 328 - protein, 107 aa, synthetic construct
  REGION 1..107: note = Synthetic Polypeptide
AIQMTQSPSSLSASVGDTVTITCRASEDIYNGLAWYQQKPGKAPKLLIYGASSLQDGVPSRFSGSGSGTDYTLTISSLQPEDEATYYCQQSRNYQQTFGGGTKVEIK

SEQ ID NO: 329 - protein, 243 aa, synthetic construct
  REGION 1..243: note = Synthetic Polypeptide
EVQLLESGGGLVQPGGSLRLSCAASGFTFSNYDMAWVRQAPGKGLEWVSSISTRGDITSYRDSVKGRFTISRDNAKNTLYLQMNSLRAEDTAVYYCARQDYYTDYMGFAYWGQGTLVTVSSGGGGSGGGGSGGGGSAIQMTQSPSSLSASVGDTVTITCRASEDIYNGLAWYQQKPGKAPKLLIYGASSLQDGVPSRFSGSGSGTDYTLTISSLQPEDEATYYCQQSRNYQQTFGGGTKVEIK

SEQ ID NO: 330 - protein, 498 aa, synthetic construct
  REGION 1..498: note = Synthetic Polypeptide
EVQLLESGGGLVQPGGSLRLSCAASGFTFSNYDMAWVRQAPGKGLEWVSSISTRGDITSYRDSVKGRFTISRDNAKNTLYLQMNSLRAEDTAVYYCARQDYYTDYMGFAYWGQGTLVTVSSGGGGSGGGGSGGGGSAIQMTQSPSSLSASVGDTVTITCRASEDIYNGLAWYQQKPGKAPKLLIYGASSLQDGVPSRFSGSGSGTDYTLTISSLQPEDEATYYCQQSRNYQQTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 331 - protein, 5 aa, synthetic construct
  REGION 1..5: note = Synthetic Polypeptide
NYDMA

SEQ ID NO: 332 - protein, 17 aa, synthetic construct
  REGION 1..17: note = Synthetic Polypeptide
SISTRGDITSYRDSVKG

SEQ ID NO: 333 - protein, 12 aa, synthetic construct
  REGION 1..12: note = Synthetic Polypeptide
QDYYTDYMGFAY

SEQ ID NO: 334 - protein, 11 aa, synthetic construct
  REGION 1..11: note = Synthetic Polypeptide
RASEDIYNGLA

SEQ ID NO: 335 - protein, 7 aa, synthetic construct
  REGION 1..7: note = Synthetic Polypeptide
GASSLQD

SEQ ID NO: 336 - protein, 9 aa, synthetic construct
  REGION 1..9: note = Synthetic Polypeptide
QQSRNYQQT

SEQ ID NO: 337 - protein, 121 aa, synthetic construct; identical to SEQ ID NO: 297
  REGION 1..121: note = Synthetic Polypeptide

SEQ ID NO: 338 - protein, 107 aa, synthetic construct
  REGION 1..107: note = Synthetic Polypeptide
AIQMTQSPSSLSASVGDRVTITCRASEDIYNGLAWYQQKPGKAPKLLIYGASSLQDGVPSRFSGSGSGTHYTLTISSLQPEDEATYYCQQSRNYQQTFGGGTKVEIK

SEQ ID NO: 339 - protein, 243 aa, synthetic construct
  REGION 1..243: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTHYTLTISSLQPEDEATYYCQQSRNYQQTFGGGTKVEIK

SEQ ID NO: 340 - protein, 498 aa, synthetic construct
  REGION 1..498: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTHYTLTISSLQPEDEATYYCQQSRNYQQTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 341 - protein, 5 aa, synthetic construct
  REGION 1..5: note = Synthetic Polypeptide
NYDMA

SEQ ID NO: 342 - protein, 17 aa, synthetic construct
  REGION 1..17: note = Synthetic Polypeptide
SISTRGDITSYRDSVKG

SEQ ID NO: 343 - protein, 12 aa, synthetic construct
  REGION 1..12: note = Synthetic Polypeptide
QDYYTDYMGFAY

SEQ ID NO: 344 - protein, 11 aa, synthetic construct
  REGION 1..11: note = Synthetic Polypeptide
RASEDIYNGLA

SEQ ID NO: 345 - protein, 7 aa, synthetic construct
  REGION 1..7: note = Synthetic Polypeptide
GASSLQD

SEQ ID NO: 346 - protein, 9 aa, synthetic construct
  REGION 1..9: note = Synthetic Polypeptide
QQSRNYQQT

SEQ ID NO: 347 - protein, 121 aa, synthetic construct; identical to SEQ ID NO: 297
  REGION 1..121: note = Synthetic Polypeptide

SEQ ID NO: 348 - protein, 107 aa, synthetic construct
  REGION 1..107: note = Synthetic Polypeptide
AIQMTQSPSSLSASVGDRVTITCRASEDIYNGLAWYQQKPGKAPKLLIYGASSLQDGVPSRFSGSGSGTEFTLTISSLQPEDEATYYCQQSRNYQQTFGGGTKVEIK

SEQ ID NO: 349 - protein, 243 aa, synthetic construct
  REGION 1..243: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTEFTLTISSLQPEDEATYYCQQSRNYQQTFGGGTKVEIK

SEQ ID NO: 350 - protein, 498 aa, synthetic construct
  REGION 1..498: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTEFTLTISSLQPEDEATYYCQQSRNYQQTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 351 - protein, 5 aa, synthetic construct
  REGION 1..5: note = Synthetic Polypeptide
NYDMA

SEQ ID NO: 352 - protein, 17 aa, synthetic construct
  REGION 1..17: note = Synthetic Polypeptide
SISTRGDITSYRDSVKG

SEQ ID NO: 353 - protein, 12 aa, synthetic construct
  REGION 1..12: note = Synthetic Polypeptide
QDYYTDYMGFAY

SEQ ID NO: 354 - protein, 11 aa, synthetic construct
  REGION 1..11: note = Synthetic Polypeptide
RASEDIYNGLA

SEQ ID NO: 355 - protein, 7 aa, synthetic construct
  REGION 1..7: note = Synthetic Polypeptide
GASSLQD

SEQ ID NO: 356 - protein, 9 aa, synthetic construct
  REGION 1..9: note = Synthetic Polypeptide
AGPHKYPLT

SEQ ID NO: 357 - protein, 121 aa, synthetic construct; identical to SEQ ID NO: 297
  REGION 1..121: note = Synthetic Polypeptide

SEQ ID NO: 358 - protein, 107 aa, synthetic construct
  REGION 1..107: note = Synthetic Polypeptide
AIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIK

SEQ ID NO: 359 - protein, 243 aa, synthetic construct
  REGION 1..243: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIK

SEQ ID NO: 360 - protein, 498 aa, synthetic construct
  REGION 1..498: note = Synthetic Polypeptide
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 361 - protein, 5 aa, synthetic construct
  REGION 1..5: note = Synthetic Polypeptide
NYDMA

SEQ ID NO: 362 - protein, 17 aa, synthetic construct
  REGION 1..17: note = Synthetic Polypeptide
SISTRGDITSYRDSVKG

SEQ ID NO: 363 - protein, 12 aa, synthetic construct
  REGION 1..12: note = Synthetic Polypeptide
QDYYTDYMGFAY

SEQ ID NO: 364 - protein, 11 aa, synthetic construct
  REGION 1..11: note = Synthetic Polypeptide
RASEDIYNGLA

SEQ ID NO: 365 - protein, 7 aa, synthetic construct
  REGION 1..7: note = Synthetic Polypeptide
GASSLQD

SEQ ID NO: 366 - protein, 9 aa, synthetic construct
  REGION 1..9: note = Synthetic Polypeptide
AGPHKYPLT

SEQ ID NO: 367 - protein, 121 aa, synthetic construct; identical to SEQ ID NO: 287
  REGION 1..121: note = Synthetic Polypeptide

SEQ ID NO: 368 - protein, 107 aa, synthetic construct
  REGION 1..107: note = Synthetic Polypeptide
AIQMTQSPSSLSASVGDRVTITCRASEDIYNGLAWYQQKPGKAPKLLIYGASSLQDGVPSRFSGSGSGTDFTLTISSMQPEDEATYYCAGPHKYPLTFGGGTKVEIK

SEQ ID NO: 369 - protein, 243 aa, synthetic construct
  REGION 1..243: note = Synthetic Polypeptide
EVQLLESGGGLVQPGRSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTDFTLTISSMQPEDEATYYCAGPHKYPLTFGGGTKVEIK

SEQ ID NO: 370 - protein, 498 aa, synthetic construct
  REGION 1..498: note = Synthetic Polypeptide
EVQLLESGGGLVQPGRSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTDFTLTISSMQPEDEATYYCAGPHKYPLTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 371 - protein, 5 aa, synthetic construct
  REGION 1..5: note = Synthetic Polypeptide
NYDMA

SEQ ID NO: 372 - protein, 17 aa, synthetic construct
  REGION 1..17: note = Synthetic Polypeptide
SISTRGDITSYRDSVKG

SEQ ID NO: 373 - protein, 12 aa, synthetic construct
  REGION 1..12: note = Synthetic Polypeptide
QDYYTDYMGFAY

SEQ ID NO: 374 - protein, 11 aa, synthetic construct
  REGION 1..11: note = Synthetic Polypeptide
RASEDIYNGLA

SEQ ID NO: 375 - protein, 7 aa, synthetic construct
  REGION 1..7: note = Synthetic Polypeptide
GASSLQD

SEQ ID NO: 376 - protein, 9 aa, synthetic construct
  REGION 1..9: note = Synthetic Polypeptide
QQSRNYQQT

SEQ ID NO: 377 - protein, 121 aa, synthetic construct; identical to SEQ ID NO: 287
  REGION 1..121: note = Synthetic Polypeptide

SEQ ID NO: 378 - protein, 107 aa, synthetic construct
  REGION 1..107: note = Synthetic Polypeptide
AIQMTQSPSSLSASVGDRVTITCRASEDIYNGLAWYQQKPGKAPKLLIYGASSLQDGVPSRFSGSGSGTDFTLTISSMQPEDEATYYCQQSRNYQQTFGGGTKVEIK

SEQ ID NO: 379 - protein, 243 aa, synthetic construct
  REGION 1..243: note = Synthetic Polypeptide
EVQLLESGGGLVQPGRSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTDFTLTISSMQPEDEATYYCQQSRNYQQTFGGGTKVEIK

SEQ ID NO: 380 - protein, 498 aa, synthetic construct
  REGION 1..498: note = Synthetic Polypeptide
EVQLLESGGGLVQPGRSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSGGGGSGGGGSGGGGSAIQMTQSPSSLSASVGDRVTITCRASEDIYNGLAWYQQKPGKAPKLLIYGASSLQDGVPSRFSGSGSGTDFTLTISSMQPEDEATYYCQQSRNYQQTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 381 - protein, 5 aa, synthetic construct
  REGION 1..5: note = Synthetic Polypeptide
NFDMA

SEQ ID NO: 382 - protein, 17 aa, synthetic construct
  REGION 1..17: note = Synthetic Polypeptide
SITTGGGDTYYADSVKG

SEQ ID NO: 383 - protein, 12 aa, synthetic construct
  REGION 1..12: note = Synthetic Polypeptide
HGYYDGYHLFDY

SEQ ID NO: 384 - protein, 11 aa, synthetic construct
  REGION 1..11: note = Synthetic Polypeptide
RASQGISNYLN

SEQ ID NO: 385 - protein, 7 aa, synthetic construct
  REGION 1..7: note = Synthetic Polypeptide
YTSNLQS

SEQ ID NO: 386 - protein, 9 aa, synthetic construct
  REGION 1..9: note = Synthetic Polypeptide
MGQTISSYT

SEQ ID NO: 387 - protein, 121 aa, synthetic construct
  REGION 1..121: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRAEDTAVYYCVRHGYYDGYHLFDYWGQGTLVTVSS

SEQ ID NO: 388 - protein, 107 aa, synthetic construct; identical to SEQ ID NO: 98
  REGION 1..107: note = Synthetic Polypeptide

SEQ ID NO: 389 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 390 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 391 - protein, 5 aa, synthetic construct
  REGION 1..5: note = Synthetic Polypeptide
NFDMA

SEQ ID NO: 392 - protein, 17 aa, synthetic construct
  REGION 1..17: note = Synthetic Polypeptide
SITTGGGDTYYADSVKG

SEQ ID NO: 393 - protein, 12 aa, synthetic construct
  REGION 1..12: note = Synthetic Polypeptide
HGYYDGYHLFDY

SEQ ID NO: 394 - protein, 11 aa, synthetic construct
  REGION 1..11: note = Synthetic Polypeptide
RASQGISNYLN

SEQ ID NO: 395 - protein, 7 aa, synthetic construct
  REGION 1..7: note = Synthetic Polypeptide
YTSNLQS

SEQ ID NO: 396 - protein, 9 aa, synthetic construct
  REGION 1..9: note = Synthetic Polypeptide
MGQTISSYT

SEQ ID NO: 397 - protein, 121 aa, synthetic construct
  REGION 1..121: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMNSLRAEDTAVYYCVRHGYYDGYHLFDYWGQGTLVTVSS

SEQ ID NO: 398 - protein, 107 aa, synthetic construct; identical to SEQ ID NO: 98
  REGION 1..107: note = Synthetic Polypeptide

SEQ ID NO: 399 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 400 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDYTLTISSLQPEDFATYYCMGQTISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 401 - protein, 5 aa, synthetic construct
  REGION 1..5: note = Synthetic Polypeptide
NFDMA

SEQ ID NO: 402 - protein, 17 aa, synthetic construct
  REGION 1..17: note = Synthetic Polypeptide
SITTGGGDTYYADSVKG

SEQ ID NO: 403 - protein, 12 aa, synthetic construct
  REGION 1..12: note = Synthetic Polypeptide
HGYYDGYHLFDY

SEQ ID NO: 404 - protein, 11 aa, synthetic construct
  REGION 1..11: note = Synthetic Polypeptide
RASQGISNYLN

SEQ ID NO: 405 - protein, 7 aa, synthetic construct
  REGION 1..7: note = Synthetic Polypeptide
YTSNLQS

SEQ ID NO: 406 - protein, 9 aa, synthetic construct
  REGION 1..9: note = Synthetic Polypeptide
MGQTISSYT

SEQ ID NO: 407 - protein, 121 aa, synthetic construct
  REGION 1..121: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRSEDTAVYYCVRHGYYDGYHLFDYWGQGTLVTVSS

SEQ ID NO: 408 - protein, 107 aa, synthetic construct; identical to SEQ ID NO: 138
  REGION 1..107: note = Synthetic Polypeptide

SEQ ID NO: 409 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRSEDTAVYYCVRHGYYDGYHLFDYWGQGTLVTVSSGGGGSGGGGSGGGGSDIQMTQSPSSLSASVGDRVTISCRASQGISNYLNWYQQKPGKAPKPLIYYTSNLQSGVPSRFSGSGSGTDYTLTISSLQPEDFATYYCMGQTISSYTFGQGTKLEIK

SEQ ID NO: 410 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRSEDTAVYYCVRHGYYDGYHLFDYWGQGTLVTVSSGGGGSGGGGSGGGGSDIQMTQSPSSLSASVGDRVTISCRASQGISNYLNWYQQKPGKAPKPLIYYTSNLQSGVPSRFSGSGSGTDYTLTISSLQPEDFATYYCMGQTISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 411 - protein, 5 aa, synthetic construct
  REGION 1..5: note = Synthetic Polypeptide
NFDMA

SEQ ID NO: 412 - protein, 17 aa, synthetic construct
  REGION 1..17: note = Synthetic Polypeptide
SITTGGGDTYYADSVKG

SEQ ID NO: 413 - protein, 12 aa, synthetic construct
  REGION 1..12: note = Synthetic Polypeptide
HGYYDGYHLFDY

SEQ ID NO: 414 - protein, 11 aa, synthetic construct
  REGION 1..11: note = Synthetic Polypeptide
RASQGISNYLN

SEQ ID NO: 415 - protein, 7 aa, synthetic construct
  REGION 1..7: note = Synthetic Polypeptide
YTSNLQS

SEQ ID NO: 416 - protein, 9 aa, synthetic construct
  REGION 1..9: note = Synthetic Polypeptide
MGQTISSYT

SEQ ID NO: 417 - protein, 121 aa, synthetic construct
  REGION 1..121: note = Synthetic Polypeptide
EVQLVESGGGLVQPGRSLRLSCAASGFTFSNFDMAWVRQAPAKGLEWVSSITTGGGDTYYADSVKGRFTISRDNAKNTLYLQMNSLRAEDTAVYYCVRHGYYDGYHLFDYWGQGTLVTVSS

SEQ ID NO: 418 - protein, 107 aa, synthetic construct; identical to SEQ ID NO: 148
  REGION 1..107: note = Synthetic Polypeptide

SEQ ID NO: 419 - protein, 243 aa, synthetic construct
  REGION 1..243: note = Synthetic Polypeptide
EVQLVESGGGLVQPGRSLRLSCAASGFTFSNFDMAWVRQAPAKGLEWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDFTLTISSLQPEDFATYYCMGQTISSYTFGQGTKLEIK

SEQ ID NO: 420 - protein, 498 aa, synthetic construct
  REGION 1..498: note = Synthetic Polypeptide
EVQLVESGGGLVQPGRSLRLSCAASGFTFSNFDMAWVRQAPAKGLEWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTITCRASQGISNYLNWYQQKPGKAPKPLIYYTSNLQSGVPSRFSGSGSGTDFTLTISSLQPEDFATYYCMGQTISSYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 421 - protein, 5 aa, synthetic construct
  REGION 1..5: note = Synthetic Polypeptide
NFDMA

SEQ ID NO: 422 - protein, 17 aa, synthetic construct
  REGION 1..17: note = Synthetic Polypeptide
SITTGGGDTYYADSVKG

SEQ ID NO: 423 - protein, 12 aa, synthetic construct
  REGION 1..12: note = Synthetic Polypeptide
HGYYDGYHLFDY

SEQ ID NO: 424 - protein, 11 aa, synthetic construct
  REGION 1..11: note = Synthetic Polypeptide
RASQGISNHLN

SEQ ID NO: 425 - protein, 7 aa, synthetic construct
  REGION 1..7: note = Synthetic Polypeptide
YTSNLQS

SEQ ID NO: 426 - protein, 9 aa, synthetic construct
  REGION 1..9: note = Synthetic Polypeptide
QQYFDRPYT

SEQ ID NO: 427 - protein, 121 aa, synthetic construct; identical to SEQ ID NO: 397
  REGION 1..121: note = Synthetic Polypeptide

SEQ ID NO: 428 - protein, 107 aa, synthetic construct
  REGION 1..107: note = Synthetic Polypeptide
DIQMTQSPSSLSASVGDRVTISCRASQGISNHLNWFQQKPGRAPKPLIYYTSNLQSGVPSRFSGSGSGTDFTLTISSLQPEDFATYYCQQYFDRPYTFGGGTKVEIK

SEQ ID NO: 429 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTISCRASQGISNHLNWFQQKPGRAPKPLIYYTSNLQSGVPSRFSGSGSGTDFTLTISSLQPEDFATYYCQQYFDRPYTFGGGTKVEIK

SEQ ID NO: 430 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTISCRASQGISNHLNWFQQKPGRAPKPLIYYTSNLQSGVPSRFSGSGSGTDFTLTISSLQPEDFATYYCQQYFDRPYTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 431 - protein, 5 aa, synthetic construct
  REGION 1..5: note = Synthetic Polypeptide
NFDMA

SEQ ID NO: 432 - protein, 17 aa, synthetic construct
  REGION 1..17: note = Synthetic Polypeptide
SITTGGGDTYYADSVKG

SEQ ID NO: 433 - protein, 12 aa, synthetic construct
  REGION 1..12: note = Synthetic Polypeptide
HGYYDGYHLFDY

SEQ ID NO: 434 - protein, 11 aa, synthetic construct
  REGION 1..11: note = Synthetic Polypeptide
RASQGISNHLN

SEQ ID NO: 435 - protein, 7 aa, synthetic construct
  REGION 1..7: note = Synthetic Polypeptide
YTSNLQS

SEQ ID NO: 436 - protein, 9 aa, synthetic construct
  REGION 1..9: note = Synthetic Polypeptide
QQYFDRPYT

SEQ ID NO: 437 - protein, 121 aa, synthetic construct; identical to SEQ ID NO: 387
  REGION 1..121: note = Synthetic Polypeptide

SEQ ID NO: 438 - protein, 107 aa, synthetic construct
  REGION 1..107: note = Synthetic Polypeptide
DIQMTQSPSSLSASVGDRVTISCRASQGISNHLNWYQQKPGKAPKPLIYYTSNLQSGVPSRFSGSGSGTDYTLTISSLQPEDFATYYCQQYFDRPYTFGGGTKVEIK

SEQ ID NO: 439 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTISCRASQGISNHLNWYQQKPGKAPKPLIYYTSNLQSGVPSRFSGSGSGTDYTLTISSLQPEDFATYYCQQYFDRPYTFGGGTKVEIK

SEQ ID NO: 440 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTISCRASQGISNHLNWYQQKPGKAPKPLIYYTSNLQSGVPSRFSGSGSGTDYTLTISSLQPEDFATYYCQQYFDRPYTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 441 - protein, 5 aa, synthetic construct
  REGION 1..5: note = Synthetic Polypeptide
NFDMA

SEQ ID NO: 442 - protein, 17 aa, synthetic construct
  REGION 1..17: note = Synthetic Polypeptide
SITTGGGDTYYADSVKG

SEQ ID NO: 443 - protein, 12 aa, synthetic construct
  REGION 1..12: note = Synthetic Polypeptide
HGYYDGYHLFDY

SEQ ID NO: 444 - protein, 11 aa, synthetic construct
  REGION 1..11: note = Synthetic Polypeptide
RASQGISNHLN

SEQ ID NO: 445 - protein, 7 aa, synthetic construct
  REGION 1..7: note = Synthetic Polypeptide
YTSNLQS

SEQ ID NO: 446 - protein, 9 aa, synthetic construct
  REGION 1..9: note = Synthetic Polypeptide
QQYFDRPYT

SEQ ID NO: 447 - protein, 121 aa, synthetic construct; identical to SEQ ID NO: 387
  REGION 1..121: note = Synthetic Polypeptide

SEQ ID NO: 448 - protein, 107 aa, synthetic construct
  REGION 1..107: note = Synthetic Polypeptide
DIQMTQSPSSLSASVGDRVTITCRASQGISNHLNWFQQKPGKAPKPLIYYTSNLQSGVPSRFSGSGSGTDFTLTISSLQPEDFATYYCQQYFDRPYTFGGGTKVEIK

SEQ ID NO: 449 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTITCRASQGISNHLNWFQQKPGKAPKPLIYYTSNLQSGVPSRFSGSGSGTDFTLTISSLQPEDFATYYCQQYFDRPYTFGGGTKVEIK

SEQ ID NO: 450 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTITCRASQGISNHLNWFQQKPGKAPKPLIYYTSNLQSGVPSRFSGSGSGTDFTLTISSLQPEDFATYYCQQYFDRPYTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 451 - protein, 5 aa, synthetic construct
  REGION 1..5: note = Synthetic Polypeptide
NFDMA

SEQ ID NO: 452 - protein, 17 aa, synthetic construct
  REGION 1..17: note = Synthetic Polypeptide
SITTGGGDTYYADSVKG

SEQ ID NO: 453 - protein, 12 aa, synthetic construct
  REGION 1..12: note = Synthetic Polypeptide
HGYYDGYHLFDY

SEQ ID NO: 454 - protein, 11 aa, synthetic construct
  REGION 1..11: note = Synthetic Polypeptide
RASQGISNHLN

SEQ ID NO: 455 - protein, 7 aa, synthetic construct
  REGION 1..7: note = Synthetic Polypeptide
YTSNLQS

SEQ ID NO: 456 - protein, 9 aa, synthetic construct
  REGION 1..9: note = Synthetic Polypeptide
QQYFDRPYT

SEQ ID NO: 457 - protein, 121 aa, synthetic construct; identical to SEQ ID NO: 417
  REGION 1..121: note = Synthetic Polypeptide

SEQ ID NO: 458 - protein, 107 aa, synthetic construct
  REGION 1..107: note = Synthetic Polypeptide
DIQMTQSPSSLSASVGERVTITCRASQGISNHLNWYQQKPGKAPKSLIYYTSNLQSGVPSRFSGSGSGTDFTLTISSLQPEDFATYYCQQYFDRPYTFGGGTKVEIK

SEQ ID NO: 459 - protein, 243 aa, synthetic construct
  REGION 1..243: note = Synthetic Polypeptide
EVQLVESGGGLVQPGRSLRLSCAASGFTFSNFDMAWVRQAPAKGLEWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGERVTITCRASQGISNHLNWYQQKPGKAPKSLIYYTSNLQSGVPSRFSGSGSGTDFTLTISSLQPEDFATYYCQQYFDRPYTFGGGTKVEIK

SEQ ID NO: 460 - protein, 498 aa, synthetic construct
  REGION 1..498: note = Synthetic Polypeptide
EVQLVESGGGLVQPGRSLRLSCAASGFTFSNFDMAWVRQAPAKGLEWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGERVTITCRASQGISNHLNWYQQKPGKAPKSLIYYTSNLQSGVPSRFSGSGSGTDFTLTISSLQPEDFATYYCQQYFDRPYTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 461 - protein, 5 aa, synthetic construct
  REGION 1..5: note = Synthetic Polypeptide
NFDMA

SEQ ID NO: 462 - protein, 17 aa, synthetic construct
  REGION 1..17: note = Synthetic Polypeptide
SITTGGGDTYYADSVKG

SEQ ID NO: 463 - protein, 12 aa, synthetic construct
  REGION 1..12: note = Synthetic Polypeptide
HGYYDGYHLFDY

SEQ ID NO: 464 - protein, 11 aa, synthetic construct
  REGION 1..11: note = Synthetic Polypeptide
RASQGISNHLN

SEQ ID NO: 465 - protein, 7 aa, synthetic construct
  REGION 1..7: note = Synthetic Polypeptide
YTSNLQS

SEQ ID NO: 466 - protein, 9 aa, synthetic construct
  REGION 1..9: note = Synthetic Polypeptide
QQYSNLPYT

SEQ ID NO: 467 - protein, 121 aa, synthetic construct; identical to SEQ ID NO: 397
  REGION 1..121: note = Synthetic Polypeptide

SEQ ID NO: 468 - protein, 107 aa, synthetic construct
  REGION 1..107: note = Synthetic Polypeptide
DIQMTQSPSSLSASVGDRVTISCRASQGISNHLNWFQQKPGRAPKPLIYYTSNLQSGVPSRFSGSGSGTDFTLTISSLQPEDFATYYCQQYSNLPYTFGGGTKVEIK

SEQ ID NO: 469 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTISCRASQGISNHLNWFQQKPGRAPKPLIYYTSNLQSGVPSRFSGSGSGTDFTLTISSLQPEDFATYYCQQYSNLPYTFGGGTKVEIK

SEQ ID NO: 470 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGDRVTISCRASQGISNHLNWFQQKPGRAPKPLIYYTSNLQSGVPSRFSGSGSGTDFTLTISSLQPEDFATYYCQQYSNLPYTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 471 - protein, 5 aa, synthetic construct
  REGION 1..5: note = Synthetic Polypeptide
NFDMA

SEQ ID NO: 472 - protein, 17 aa, synthetic construct
  REGION 1..17: note = Synthetic Polypeptide
SITTGGGDTYYADSVKG

SEQ ID NO: 473 - protein, 12 aa, synthetic construct
  REGION 1..12: note = Synthetic Polypeptide
HGYYDGYHLFDY

SEQ ID NO: 474 - protein, 11 aa, synthetic construct
  REGION 1..11: note = Synthetic Polypeptide
RASQGISNHLN

SEQ ID NO: 475 - protein, 7 aa, synthetic construct
  REGION 1..7: note = Synthetic Polypeptide
YTSNLQS

SEQ ID NO: 476 - protein, 9 aa, synthetic construct
  REGION 1..9: note = Synthetic Polypeptide
QQYSNLPYT

SEQ ID NO: 477 - protein, 121 aa, synthetic construct; identical to SEQ ID NO: 387
  REGION 1..121: note = Synthetic Polypeptide

SEQ ID NO: 478 - protein, 107 aa, synthetic construct
  REGION 1..107: note = Synthetic Polypeptide
DIQMTQSPSSLSASVGDRVTISCRASQGISNHLNWYQQKPGKAPKPLIYYTSNLQSGVPSRFSGSGSGTDYTLTISSLQPEDFATYYCQQYSNLPYTFGGGTKVEIK

SEQ ID NO: 479 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTISCRASQGISNHLNWYQQKPGKAPKPLIYYTSNLQSGVPSRFSGSGSGTDYTLTISSLQPEDFATYYCQQYSNLPYTFGGGTKVEIK

SEQ ID NO: 480 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRAEDTAVYYCVRHGYYDGYHLFDYWGQGTLVTVSSGGGGSGGGGSGGGGSDIQMTQSPSSLSASVGDRVTISCRASQGISNHLNWYQQKPGKAPKPLIYYTSNLQSGVPSRFSGSGSGTDYTLTISSLQPEDFATYYCQQYSNLPYTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 481 - protein, 5 aa, synthetic construct
  REGION 1..5: note = Synthetic Polypeptide
NFDMA

SEQ ID NO: 482 - protein, 17 aa, synthetic construct
  REGION 1..17: note = Synthetic Polypeptide
SITTGGGDTYYADSVKG

SEQ ID NO: 483 - protein, 12 aa, synthetic construct
  REGION 1..12: note = Synthetic Polypeptide
HGYYDGYHLFDY

SEQ ID NO: 484 - protein, 11 aa, synthetic construct
  REGION 1..11: note = Synthetic Polypeptide
RASQGISNHLN

SEQ ID NO: 485 - protein, 7 aa, synthetic construct
  REGION 1..7: note = Synthetic Polypeptide
YTSNLQS

SEQ ID NO: 486 - protein, 9 aa, synthetic construct
  REGION 1..9: note = Synthetic Polypeptide
QQYSNLPYT

SEQ ID NO: 487 - protein, 121 aa, synthetic construct; identical to SEQ ID NO: 417
  REGION 1..121: note = Synthetic Polypeptide

SEQ ID NO: 488 - protein, 107 aa, synthetic construct
  REGION 1..107: note = Synthetic Polypeptide
DIQMTQSPSSLSASVGERVTITCRASQGISNHLNWYQQKPGKAPKSLIYYTSNLQSGVPSRFSGSGSGTDFTLTISSLQPEDFATYYCQQYSNLPYTFGGGTKVEIK

SEQ ID NO: 489 - protein, 243 aa, synthetic construct
  REGION 1..243: note = Synthetic Polypeptide
EVQLVESGGGLVQPGRSLRLSCAASGFTFSNFDMAWVRQAPAKGLEWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGERVTITCRASQGISNHLNWYQQKPGKAPKSLIYYTSNLQSGVPSRFSGSGSGTDFTLTISSLQPEDFATYYCQQYSNLPYTFGGGTKVEIK

SEQ ID NO: 490 - protein, 498 aa, synthetic construct
  REGION 1..498: note = Synthetic Polypeptide
EVQLVESGGGLVQPGRSLRLSCAASGFTFSNFDMAWVRQAPAKGLEWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSLSASVGERVTITCRASQGISNHLNWYQQKPGKAPKSLIYYTSNLQSGVPSRFSGSGSGTDFTLTISSLQPEDFATYYCQQYSNLPYTFGGGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 491 - protein, 5 aa, synthetic construct
  REGION 1..5: note = Synthetic Polypeptide
NFDMA

SEQ ID NO: 492 - protein, 17 aa, synthetic construct
  REGION 1..17: note = Synthetic Polypeptide
SITTGGGDTYYADSVKG

SEQ ID NO: 493 - protein, 12 aa, synthetic construct
  REGION 1..12: note = Synthetic Polypeptide
HGYYDGYHLFDY

SEQ ID NO: 494 - protein, 11 aa, synthetic construct
  REGION 1..11: note = Synthetic Polypeptide
RASQGISNNLN

SEQ ID NO: 495 - protein, 7 aa, synthetic construct
  REGION 1..7: note = Synthetic Polypeptide
YTSNLQS

SEQ ID NO: 496 - protein, 9 aa, synthetic construct
  REGION 1..9: note = Synthetic Polypeptide
QQFTSLPYT

SEQ ID NO: 497 - protein, 121 aa, synthetic construct; identical to SEQ ID NO: 397
  REGION 1..121: note = Synthetic Polypeptide

SEQ ID NO: 498 - protein, 107 aa, synthetic construct
  REGION 1..107: note = Synthetic Polypeptide
DIQMTQSPSSMSASVGDRVTITCRASQGISNNLNWYQQKPGKAPKSLIYYTSNLQSGVPSRFSGSGSGTDYTLTISSLQPEDFATYYCQQFTSLPYTFGQGTKLEIK

SEQ ID NO: 499 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSMSASVGDRVTITCRASQGISNNLNWYQQKPGKAPKSLIYYTSNLQSGVPSRFSGSGSGTDYTLTISSLQPEDFATYYCQQFTSLPYTFGQGTKLEIK

SEQ ID NO: 500 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMNSLRAEDTAVYYCVRHGYYDGYHLFDYWGQGTLVTVSSGGGGSGGGGSGGGGSDIQMTQSPSSMSASVGDRVTITCRASQGISNNLNWYQQKPGKAPKSLIYYTSNLQSGVPSRFSGSGSGTDYTLTISSLQPEDFATYYCQQFTSLPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 501 - protein, 5 aa, synthetic construct
  REGION 1..5: note = Synthetic Polypeptide
NFDMA

SEQ ID NO: 502 - protein, 17 aa, synthetic construct
  REGION 1..17: note = Synthetic Polypeptide
SITTGGGDTYYADSVKG

SEQ ID NO: 503 - protein, 12 aa, synthetic construct
  REGION 1..12: note = Synthetic Polypeptide
HGYYDGYHLFDY

SEQ ID NO: 504 - protein, 11 aa, synthetic construct
  REGION 1..11: note = Synthetic Polypeptide
RANQGISNNLN

SEQ ID NO: 505 - protein, 7 aa, synthetic construct
  REGION 1..7: note = Synthetic Polypeptide
YTSNLQS

SEQ ID NO: 506 - protein, 9 aa, synthetic construct
  REGION 1..9: note = Synthetic Polypeptide
QQFTSLPYT

SEQ ID NO: 507 - protein, 121 aa, synthetic construct
  REGION 1..121: note = Synthetic Polypeptide
EVQLVESGGGLVQPGGSLRLSCAASGFTFSNFDMAWVRQAPGKGLVWVSSITTGGGDTYYADSVKGRFTISRDNAKSTLYLQMDSLRSEDTAVYYCVRHGYYDGYHLFDYWGQGTLVTVSS

SEQ ID NO: 508 - protein, 107 aa, synthetic construct
  REGION 1..107: note = Synthetic Polypeptide
DIQMTQSPSSLSASVGDRVTITCRANQGISNNLNWYQQKPGKAPKPLIYYTSNLQSGVPSRFSGSGSGTDYTLTISSLQPEDFATYYCQQFTSLPYTFGQGTKLEIK

SEQ ID NO: 509 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKSTLYLQMDSLRSEDTAVYYCVRHGYYDGYHLFDYWGQGTLVTVSSGGGGSGGGGSGGGGSDIQMTQSPSSLSASVGDRVTITCRANQGISNNLNWYQQKPGKAPKPLIYYTSNLQSGVPSRFSGSGSGTDYTLTISSLQPEDFATYYCQQFTSLPYTFGQGTKLEIK

SEQ ID NO: 510 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKSTLYLQMDSLRSEDTAVYYCVRHGYYDGYHLFDYWGQGTLVTVSSGGGGSGGGGSGGGGSDIQMTQSPSSLSASVGDRVTITCRANQGISNNLNWYQQKPGKAPKPLIYYTSNLQSGVPSRFSGSGSGTDYTLTISSLQPEDFATYYCQQFTSLPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 511 - protein, 5 aa, synthetic construct
  REGION 1..5: note = Synthetic Polypeptide
NFDMA

SEQ ID NO: 512 - protein, 17 aa, synthetic construct
  REGION 1..17: note = Synthetic Polypeptide
SITTGGGDTYYADSVKG

SEQ ID NO: 513 - protein, 12 aa, synthetic construct
  REGION 1..12: note = Synthetic Polypeptide
HGYYDGYHLFDY

SEQ ID NO: 514 - protein, 11 aa, synthetic construct
  REGION 1..11: note = Synthetic Polypeptide
RASQGISNNLN

SEQ ID NO: 515 - protein, 7 aa, synthetic construct
  REGION 1..7: note = Synthetic Polypeptide
YTSNLQS

SEQ ID NO: 516 - protein, 9 aa, synthetic construct
  REGION 1..9: note = Synthetic Polypeptide
QQFTSLPYT

SEQ ID NO: 517 - protein, 121 aa, synthetic construct; identical to SEQ ID NO: 407
  REGION 1..121: note = Synthetic Polypeptide

SEQ ID NO: 518 - protein, 107 aa, synthetic construct; identical to SEQ ID NO: 498
  REGION 1..107: note = Synthetic Polypeptide

SEQ ID NO: 519 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRSEDTAVYYCVRHGYYDGYHLFDYWGQGTLVTVSSGGGGSGGGGSGGGGSDIQMTQSPSSMSASVGDRVTITCRASQGISNNLNWYQQKPGKAPKSLIYYTSNLQSGVPSRFSGSGSGTDYTLTISSLQPEDFATYYCQQFTSLPYTFGQGTKLEIK

SEQ ID NO: 520 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRSEDTAVYYCVRHGYYDGYHLFDYWGQGTLVTVSSGGGGSGGGGSGGGGSDIQMTQSPSSMSASVGDRVTITCRASQGISNNLNWYQQKPGKAPKSLIYYTSNLQSGVPSRFSGSGSGTDYTLTISSLQPEDFATYYCQQFTSLPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 521 - protein, 5 aa, synthetic construct
  REGION 1..5: note = Synthetic Polypeptide
NFDMA

SEQ ID NO: 522 - protein, 17 aa, synthetic construct
  REGION 1..17: note = Synthetic Polypeptide
SITTGGGDTYYADSVKG

SEQ ID NO: 523 - protein, 12 aa, synthetic construct
  REGION 1..12: note = Synthetic Polypeptide
HGYYDGYHLFDY

SEQ ID NO: 524 - protein, 11 aa, synthetic construct
  REGION 1..11: note = Synthetic Polypeptide
RASQGISNNLN

SEQ ID NO: 525 - protein, 7 aa, synthetic construct
  REGION 1..7: note = Synthetic Polypeptide
YTSNLQS

SEQ ID NO: 526 - protein, 9 aa, synthetic construct
  REGION 1..9: note = Synthetic Polypeptide
QQFAHLPYT

SEQ ID NO: 527 - protein, 121 aa, synthetic construct; identical to SEQ ID NO: 407
  REGION 1..121: note = Synthetic Polypeptide

SEQ ID NO: 528 - protein, 107 aa, synthetic construct
  REGION 1..107: note = Synthetic Polypeptide
DIQMTQSPSSMSASVGDRVTITCRASQGISNNLNWYQQKPGKAPKSLIYYTSNLQSGVPSRFSGSGSGTDYTLTISSLQPEDFATYYCQQFAHLPYTFGQGTKLEIK

SEQ ID NO: 529 - protein, 243 aa, synthetic construct
  REGION 1..243: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRSEDTAVYYCVRHGYYDGYHLFDYWGQGTLVTVSSGGGGSGGGGSGGGGSDIQMTQSPSSMSASVGDRVTITCRASQGISNNLNWYQQKPGKAPKSLIYYTSNLQSGVPSRFSGSGSGTDYTLTISSLQPEDFATYYCQQFAHLPYTFGQGTKLEIK

SEQ ID NO: 530 - protein, 498 aa, synthetic construct
  REGION 1..498: note = Synthetic Polypeptide
EVQLVESGGGLVQPGGSLRLSCAASGFTFSNFDMAWVRQAPGKGLVWVSSITTGGGDTYYADSVKGRFTISRDNAKNTLYLQMDSLRSEDTAVYYCVRHGYYDGYHLFDYWGQGTLVTVSSGGGGSGGGGSGGGGSDIQMTQSPSSMSASVGDRVTITCRASQGISNNLNWYQQKPGKAPKSLIYYTSNLQSGVPSRFSGSGSGTDYTLTISSLQPEDFATYYCQQFAHLPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 531 - protein, 14 aa, synthetic construct
  REGION 1..14: note = Synthetic Polypeptide
GSSTGAVTSGYYPN

SEQ ID NO: 532 - protein, 7 aa, synthetic construct
  REGION 1..7: note = Synthetic Polypeptide
GTKFLAP

SEQ ID NO: 533 - protein, 9 aa, synthetic construct
  REGION 1..9: note = Synthetic Polypeptide
ALWYSNRWV

SEQ ID NO: 534 - protein, 5 aa, synthetic construct
  REGION 1..5: note = Synthetic Polypeptide
IYAMN

SEQ ID NO: 535 - protein, 19 aa, synthetic construct
  REGION 1..19: note = Synthetic Polypeptide
RIRSKYNNYATYYADSVKS

SEQ ID NO: 536 - protein, 14 aa, synthetic construct
  REGION 1..14: note = Synthetic Polypeptide
HGNFGNSYVSFFAY

SEQ ID NO: 537 - protein, 125 aa, synthetic construct
  REGION 1..125: note = Synthetic Polypeptide
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 538 - protein, 125 aa, synthetic construct
  REGION 1..125: note = Synthetic Polypeptide
EVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 539 - protein, 109 aa, synthetic construct
  REGION 1..109: note = Synthetic Polypeptide
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 540 - protein, 249 aa, synthetic construct
  REGION 1..249: note = Synthetic Polypeptide
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 541 - protein, 249 aa, synthetic construct
  REGION 1..249: note = Synthetic Polypeptide
EVQLLESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 542 - protein, 14 aa, synthetic construct
  REGION 1..14: note = Synthetic Polypeptide
GSSTGAVTSGYYPN

SEQ ID NO: 543 - protein, 7 aa, synthetic construct
  REGION 1..7: note = Synthetic Polypeptide
GTKFLAP

SEQ ID NO: 544 - protein, 9 aa, synthetic construct
  REGION 1..9: note = Synthetic Polypeptide
ALWYSNRWV

SEQ ID NO: 545 - protein, 5 aa, synthetic construct
  REGION 1..5: note = Synthetic Polypeptide
KYAMN

SEQ ID NO: 546 - protein, 19 aa, synthetic construct
  REGION 1..19: note = Synthetic Polypeptide
RIRSKYNNYATYYADSVKD

SEQ ID NO: 547 - protein, 14 aa, synthetic construct
  REGION 1..14: note = Synthetic Polypeptide
HGNFGNSYISYWAY

SEQ ID NO: 548 - protein, 125 aa, synthetic construct
  REGION 1..125: note = Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 549 - protein, 125 aa, synthetic construct
  REGION 1..125: note = Synthetic Polypeptide
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 550 - protein, 109 aa, synthetic construct; identical to SEQ ID NO: 539
  REGION 1..109: note = Synthetic Polypeptide

SEQ ID NO: 551 - protein, 109 aa, synthetic construct
  REGION 1..109: note = Synthetic Polypeptide
ELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 552 - protein, 249 aa, synthetic construct
  REGION 1..249: note = Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 553 - protein, 249 aa, synthetic construct
  REGION 1..249: note = Synthetic Polypeptide
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 554 - protein, 14 aa, synthetic construct
  REGION 1..14: note = Synthetic Polypeptide
GSSTGAVTSGYYPN

SEQ ID NO: 555 - protein, 7 aa, synthetic construct
  REGION 1..7: note = Synthetic Polypeptide
GTKFLAP

SEQ ID NO: 556 - protein, 9 aa, synthetic construct
  REGION 1..9: note = Synthetic Polypeptide
ALWYSNRWV

SEQ ID NO: 557 - protein, 5 aa, synthetic construct
  REGION 1..5: note = Synthetic Polypeptide
SYAMN

SEQ ID NO: 558 - protein, 19 aa, synthetic construct
  REGION 1..19: note = Synthetic Polypeptide
RIRSKYNNYATYYADSVKG

SEQ ID NO: 559 - protein, 14 aa, synthetic construct
  REGION 1..14: note = Synthetic Polypeptide
HGNFGNSYLSFWAY

SEQ ID NO: 560 - protein, 125 aa, synthetic construct
  REGION 1..125: note = Synthetic Polypeptide
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 561 - protein, 125 aa, synthetic construct
  REGION 1..125: note = Synthetic Polypeptide
EVQLLESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 562 - protein, 109 aa, synthetic construct; identical to SEQ ID NO: 539
  REGION 1..109: note = Synthetic Polypeptide

SEQ ID NO: 563 - protein, 249 aa, synthetic construct
  REGION 1..249: note = Synthetic Polypeptide
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 564 - protein, 249 aa, synthetic construct
  REGION 1..249: note = Synthetic Polypeptide
EVQLLESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 565 - protein, 14 aa, synthetic construct
  REGION 1..14: note = Synthetic Polypeptide
GSSTGAVTSGYYPN

SEQ ID NO: 566 - protein, 7 aa, synthetic construct
  REGION 1..7: note = Synthetic Polypeptide
GTKFLAP

SEQ ID NO: 567 - protein, 9 aa, synthetic construct
  REGION 1..9: note = Synthetic Polypeptide
ALWYSNRWV

SEQ ID NO: 568 - protein, 5 aa, synthetic construct
  REGION 1..5: note = Synthetic Polypeptide
RYAMN

SEQ ID NO: 569 - protein, 19 aa, synthetic construct
  REGION 1..19: note = Synthetic Polypeptide
RIRSKYNNYATYYADSVKG

SEQ ID NO: 570 - protein, 14 aa, synthetic construct
  REGION 1..14: note = Synthetic Polypeptide
HGNFGNSYLSYFAY

SEQ ID NO: 571 - protein, 125 aa, synthetic construct
  REGION 1..125: note = Synthetic Polypeptide
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 572 - protein, 125 aa, synthetic construct
  REGION 1..125: note = Synthetic Polypeptide
EVQLLESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 573 - protein, 109 aa, synthetic construct; identical to SEQ ID NO: 539
  REGION 1..109: note = Synthetic Polypeptide

SEQ ID NO: 574 - protein, 249 aa, synthetic construct
  REGION 1..249: note = Synthetic Polypeptide
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 575 - protein, 249 aa, synthetic construct
  REGION 1..249: note = Synthetic Polypeptide
EVQLLESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 576 - protein, 14 aa, synthetic construct
  REGION 1..14: note = Synthetic Polypeptide
RSSTGAVTSGYYPN

SEQ ID NO: 577 - protein, 7 aa, synthetic construct
  REGION 1..7: note = Synthetic Polypeptide
ATDMRPS

SEQ ID NO: 578 - protein, 9 aa, synthetic construct
  REGION 1..9: note = Synthetic Polypeptide
ALWYSNRWV

SEQ ID NO: 579 - protein, 5 aa, synthetic construct
  REGION 1..5: note = Synthetic Polypeptide
VYAMN

SEQ ID NO: 580 - protein, 19 aa, synthetic construct
  REGION 1..19: note = Synthetic Polypeptide
RIRSKYNNYATYYADSVKK

SEQ ID NO: 581 - protein, 14 aa, synthetic construct
  REGION 1..14: note = Synthetic Polypeptide
HGNFGNSYLSWWAY

SEQ ID NO: 582 - protein, 125 aa, synthetic construct
  REGION 1..125: note = Synthetic Polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 583 - protein, 125 aa, synthetic construct
  REGION 1..125: note = Synthetic Polypeptide
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 584 - protein, 109 aa, synthetic construct
  REGION 1..109: note = Synthetic Polypeptide
QTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 585 - protein, 109 aa, synthetic construct
  REGION 1..109: note = Synthetic Polypeptide
ELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 586 - protein, 249 aa, synthetic construct
  REGION 1..249: note = Synthetic Polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 587 - protein, 249 aa, synthetic construct
  REGION 1..249: note = Synthetic Polypeptide
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 588 - protein, 14 aa, synthetic construct
  REGION 1..14: note = Synthetic Polypeptide
GSSTGAVTSGYYPN

SEQ ID NO: 589 - protein, 7 aa, synthetic construct
  REGION 1..7: note = Synthetic Polypeptide
GTKFLAP

SEQ ID NO: 590 - protein, 9 aa, synthetic construct
  REGION 1..9: note = Synthetic Polypeptide
ALWYSNRWV

SEQ ID NO: 591 - protein, 5 aa, synthetic construct
  REGION 1..5: note = Synthetic Polypeptide
KYAMN

SEQ ID NO: 592 - protein, 19 aa, synthetic construct
  REGION 1..19: note = Synthetic Polypeptide
RIRSKYNNYATYYADSVKS

SEQ ID NO: 593 - protein, 14 aa, synthetic construct
  REGION 1..14: note = Synthetic Polypeptide
HGNFGNSYTSYYAY

SEQ ID NO: 594 - protein, 125 aa, synthetic construct
  REGION 1..125: note = Synthetic Polypeptide
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 595 - protein, 125 aa, synthetic construct
  REGION 1..125: note = Synthetic Polypeptide
EVQLLESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 596 - protein, 109 aa, synthetic construct; identical to SEQ ID NO: 539
  REGION 1..109: note = Synthetic Polypeptide

SEQ ID NO: 597 - protein, 249 aa, synthetic construct
  REGION 1..249: note = Synthetic Polypeptide
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 598 - protein, 249 aa, synthetic construct
  REGION 1..249: note = Synthetic Polypeptide
EVQLLESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 599 - protein, 14 aa, synthetic construct
  REGION 1..14: note = Synthetic Polypeptide
RSSTGAVTSGYYPN

SEQ ID NO: 600 - protein, 7 aa, synthetic construct
  REGION 1..7: note = Synthetic Polypeptide
ATDMRPS

SEQ ID NO: 601 - protein, 9 aa, synthetic construct
  REGION 1..9: note = Synthetic Polypeptide
ALWYSNRWV

SEQ ID NO: 602 - protein, 5 aa, synthetic construct
  REGION 1..5: note = Synthetic Polypeptide
GYAMN

SEQ ID NO: 603 - protein, 19 aa, synthetic construct
  REGION 1..19: note = Synthetic Polypeptide
RIRSKYNNYATYYADSVKE

SEQ ID NO: 604 - protein, 14 aa, synthetic construct
  REGION 1..14: note = Synthetic Polypeptide
HRNFGNSYLSWFAY

SEQ ID NO: 605 - protein, 125 aa, synthetic construct
  REGION 1..125: note = Synthetic Polypeptide
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 606 - protein, 125 aa, synthetic construct
  REGION 1..125: note = Synthetic Polypeptide
EVQLLESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 607 - protein, 109 aa, synthetic construct; identical to SEQ ID NO: 584
  REGION 1..109: note = Synthetic Polypeptide

SEQ ID NO: 608 - protein, 249 aa, synthetic construct
  REGION 1..249: note = Synthetic Polypeptide
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 609 - protein, 249 aa, synthetic construct
  REGION 1..249: note = Synthetic Polypeptide
EVQLLESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSELVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 610 - protein, 14 aa, synthetic construct
  REGION 1..14: note = Synthetic Polypeptide
GSSTGAVTSGYYPN

SEQ ID NO: 611 - protein, 7 aa, synthetic construct
  REGION 1..7: note = Synthetic Polypeptide
GTKFLAP

SEQ ID NO: 612 - protein, 9 aa, synthetic construct
  REGION 1..9: note = Synthetic Polypeptide
ALWYSNRWV

SEQ ID NO: 613 - protein, 5 aa, synthetic construct
  REGION 1..5: note = Synthetic Polypeptide
VYAMN

SEQ ID NO: 614 - protein, 19 aa, synthetic construct
  REGION 1..19: note = Synthetic Polypeptide
RIRSKYNNYATYYADSVKK

SEQ ID NO: 615 - protein, 14 aa, synthetic construct
  REGION 1..14: note = Synthetic Polypeptide
HGNFGNSYISWWAY

SEQ ID NO: 616 - protein, 125 aa, synthetic construct
  REGION 1..125: note = Synthetic Polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 617 - protein, 125 aa, synthetic construct
  REGION 1..125: note = Synthetic Polypeptide
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 618 - protein, 109 aa, synthetic construct; identical to SEQ ID NO: 539
  REGION 1..109: note = Synthetic Polypeptide

SEQ ID NO: 619 - protein, 249 aa, synthetic construct
  REGION 1..249: note = Synthetic Polypeptide
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 620 - protein, 249 aa, synthetic construct
  REGION 1..249: note = Synthetic Polypeptide
EVQLLESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSELVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 621 - protein, 14 aa, synthetic construct
  REGION 1..14: note = Synthetic Polypeptide
GSSTGAVTSGNYPN

SEQ ID NO: 622 - protein, 7 aa, synthetic construct
  REGION 1..7: note = Synthetic Polypeptide
GTKFLAP

SEQ ID NO: 623 - protein, 9 aa, synthetic construct
  REGION 1..9: note = Synthetic Polypeptide
VLWYSNRWV

SEQ ID NO: 624 - protein, 5 aa, synthetic construct
  REGION 1..5: note = Synthetic Polypeptide
SYAMN

SEQ ID NO: 625 - protein, 19 aa, synthetic construct
  REGION 1..19: note = Synthetic Polypeptide
RIRSKYNNYATYYADSVKG

SEQ ID NO: 626 - protein, 14 aa, synthetic construct
  REGION 1..14: note = Synthetic Polypeptide
HGNFGNSYVSWWAY

SEQ ID NO: 627 - protein, 125 aa, synthetic construct
  REGION 1..125: note = Synthetic Polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 628 - protein, 125 aa, synthetic construct
  REGION 1..125: note = Synthetic Polypeptide
EVQLLESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 629 - protein, 109 aa, synthetic construct
  REGION 1..109: note = Synthetic Polypeptide
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 630 - protein, 109 aa, synthetic construct
  REGION 1..109: note = Synthetic Polypeptide
ELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 631 - protein, 249 aa, synthetic construct
  REGION 1..249: note = Synthetic Polypeptide
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 632 - protein, 249 aa, synthetic construct
  REGION 1..249: note = Synthetic Polypeptide
EVQLLESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 633 - protein, 14 aa, synthetic construct
  REGION 1..14: note = Synthetic Polypeptide
GSSTGAVTSGNYPN

SEQ ID NO: 634 - protein, 7 aa, synthetic construct
  REGION 1..7: note = Synthetic Polypeptide
GTKFLAP

SEQ ID NO: 635 - protein, 9 aa, synthetic construct
  REGION 1..9: note = Synthetic Polypeptide
VLWYSNRWV

SEQ ID NO: 636 - protein, 5 aa, synthetic construct
  REGION 1..5: note = Synthetic Polypeptide
KYAMN

SEQ ID NO: 637 - protein, 19 aa, synthetic construct
  REGION 1..19: note = Synthetic Polypeptide
RIRSKYNNYATYYADSVKD

SEQ ID NO: 638 - protein, 14 aa, synthetic construct
  REGION 1..14: note = Synthetic Polypeptide
HGNFGNSYISYWAY

SEQ ID NO: 639 - protein, 125 aa, synthetic construct; identical to SEQ ID NO: 548
  REGION 1..125: note = Synthetic Polypeptide

SEQ ID NO: 640 - protein, 125 aa, synthetic construct; identical to SEQ ID NO: 549
  REGION 1..125: note = Synthetic Polypeptide

SEQ ID NO: 641 - protein, 109 aa, synthetic construct; identical to SEQ ID NO: 629
  REGION 1..109: note = Synthetic Polypeptide

SEQ ID NO: 642 - protein, 249 aa, synthetic construct
  REGION 1..249: note = Synthetic Polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 643 - protein, 249 aa, synthetic construct
  REGION 1..249: note = Synthetic Polypeptide
EVQLLESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSELVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 644 - protein, 125 aa, synthetic construct
  REGION 1..125: note = Synthetic Polypeptide
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSS

SEQ ID NO: 645 - protein, 109 aa, synthetic construct; identical to SEQ ID NO: 629
  REGION 1..109: note = Synthetic Polypeptide

SEQ ID NO: 646 - protein, 249 aa, synthetic construct
  REGION 1..249: note = Synthetic Polypeptide
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 647 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 648 - protein, 183 aa, Homo sapiens
MLQMARQCSQNEYFDSLLHDCKPCQLRCSSTPPLTCQRYCNASMTNSVKGMNAILWTCLGLSLIISLAVFVLTFLLRKMSSEPLKDEFKNTGSGLLGMANIDLEKGRTGDEIVLPRGLEYTVEECTCEDCIKNKPKVDSDHCFPLPAMEEGATILVTTKTNDYCNSLSAALSVTEIEKSISAR

SEQ ID NO: 649 - protein, 54 aa, Homo sapiens
MLQMAGQCSQNEYFDSLLHACIPCQLRCSSNTPPLTCQRYCNASVTNSVKGTNA

SEQ ID NO: 650 - protein, 53 aa, Homo sapiens
MLQMARQCSQNEYFDSLLHDCKPCQLRCSSTPPLTCQRYCNASMTNSVKGMNA

SEQ ID NO: 651 - protein, 18 aa, Homo sapiens
CQLRCSSNTPPLTCQRYC

SEQ ID NO: 652 - protein, 17 aa, Homo sapiens
CQLRCSSTPPLTCQRYC

SEQ ID NO: 653 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 654 - protein, 27 aa, Homo sapiens
QDGNEEMGGITQTPYKVSISGTTVILT

SEQ ID NO: 655 - protein, 96 aa, Callithrix jacchus
QDGNEEMGDTTQNPYKVSISGTTVTLTCPRYDGHEIKWLVNSQNKEGHEDHLLLEDFSEMEQSGYYACLSKETPAEEASHYLYLKARVCENCVEVD

SEQ ID NO: 656 - protein, 27 aa, Callithrix jacchus
QDGNEEMGDTTQNPYKVSISGTTVTLT

SEQ ID NO: 657 - protein, 96 aa, Saguinus oedipus; identical to SEQ ID NO: 655

SEQ ID NO: 658 - protein, 27 aa, Saguinus oedipus
QDGNEEMGDTTQNPYKVSISGTTVTLT

SEQ ID NO: 659 - protein, 96 aa, Saimiri sciureus
QDGNEEIGDTTQNPYKVSISGTTVTLTCPRYDGQEIKWLVNDQNKEGHEDHLLLEDFSEMEQSGYYACLSKETPTEEASHYLYLKARVCENCVEVD

SEQ ID NO: 660 - protein, 27 aa, Saimiri sciureus
QDGNEEIGDTTQNPYKVSISGTTVTLT

SEQ ID NO: 661 - protein, 504 aa, synthetic construct
  REGION 1..504: note = Synthetic Polypeptide
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 662 - protein, 5 aa, synthetic construct
  REGION 1..5: note = Synthetic Polypeptide
HHHHH

SEQ ID NO: 663 - protein, 6 aa, synthetic construct
  REGION 1..6: note = Synthetic Polypeptide
HHHHHH

SEQ ID NO: 664 - protein, 7 aa, synthetic construct
  REGION 1..7: note = Synthetic Polypeptide
HHHHHHH

SEQ ID NO: 665 - protein, 8 aa, synthetic construct
  REGION 1..8: note = Synthetic Polypeptide
HHHHHHHH

SEQ ID NO: 666 - protein, 6 aa, synthetic construct
  REGION 1..6: note = Synthetic Polypeptide
DTYRYI

SEQ ID NO: 667 - protein, 6 aa, synthetic construct
  REGION 1..6: note = Synthetic Polypeptide
TDFYLK

SEQ ID NO: 668 - protein, 11 aa, synthetic construct
  REGION 1..11: note = Synthetic Polypeptide
MASMTGGQQMG

SEQ ID NO: 669 - protein, 14 aa, synthetic construct
  REGION 1..14: note = Synthetic Polypeptide
GKPIPNPLLGLDST

SEQ ID NO: 670 - protein, 6 aa, synthetic construct
  REGION 1..6: note = Synthetic Polypeptide
QYPALT

SEQ ID NO: 671 - protein, 10 aa, synthetic construct
  REGION 1..10: note = Synthetic Polypeptide
SSTSSDFRDR

SEQ ID NO: 672 - protein, 7 aa, synthetic construct
  REGION 1..7: note = Synthetic Polypeptide
DYKDDDK

SEQ ID NO: 673 - protein, 6 aa, synthetic construct
  REGION 1..6: note = Synthetic Polypeptide
EYMPME

SEQ ID NO: 674 - protein, 6 aa, synthetic construct
  REGION 1..6: note = Synthetic Polypeptide
EFMPME

SEQ ID NO: 675 - protein, 19 aa, synthetic construct
  REGION 1..19: note = Synthetic Polypeptide
KDHLIHNVHKEFHAHAHNK

SEQ ID NO: 676 - protein, 8 aa, synthetic construct
  REGION 1..8: note = Synthetic Polypeptide
QPELAPED

SEQ ID NO: 677 - protein, 11 aa, synthetic construct
  REGION 1..11: note = Synthetic Polypeptide
KPPTPPPEPET

SEQ ID NO: 678 - protein, 11 aa, synthetic construct
  REGION 1..11: note = Synthetic Polypeptide
CEQKLISEEDL

SEQ ID NO: 679 - protein, 9 aa, synthetic construct
  REGION 1..9: note = Synthetic Polypeptide
NANNPDWDF

SEQ ID NO: 680 - protein, 15 aa, synthetic construct
  REGION 1..15: note = Synthetic Polypeptide
KETAAAKFERQHMDS

SEQ ID NO: 681 - protein, 8 aa, synthetic construct
  REGION 1..8: note = Synthetic Polypeptide
WSHPQFEK

SEQ ID NO: 682 - protein, 9 aa, synthetic construct
  REGION 1..9: note = Synthetic Polypeptide
AWAHPQPGG

SEQ ID NO: 683 - protein, 6 aa, synthetic construct
  REGION 1..6: note = Synthetic Polypeptide
HTTPHH

SEQ ID NO: 684 - protein, 11 aa, synthetic construct
  REGION 1..11: note = Synthetic Polypeptide
YTDIEMNRLGK

SEQ ID NO: 685 - protein, 12 aa, synthetic construct
  REGION 1..12: note = Synthetic Polypeptide
EDQVDPRLIDGK

SEQ ID NO: 686 - protein, 4 aa, synthetic construct
  REGION 1..4: note = Synthetic Polypeptide
GGGG

SEQ ID NO: 687 - protein, 5 aa, synthetic construct
  REGION 1..5: note = Synthetic Polypeptide
GGGGS

SEQ ID NO: 688 - protein, 5 aa, synthetic construct
  REGION 1..5: note = Synthetic Polypeptide
GGGGQ

SEQ ID NO: 689 - protein, 6 aa, synthetic construct
  REGION 1..6: note = Synthetic Polypeptide
SGGGGS

SEQ ID NO: 690 - protein, 6 aa, synthetic construct
  REGION 1..6: note = Synthetic Polypeptide
PGGGGS

SEQ ID NO: 691 - protein, 6 aa, synthetic construct
  REGION 1..6: note = Synthetic Polypeptide
PGGDGS

SEQ ID NO: 692 - protein, 9 aa, synthetic construct
  REGION 1..9: note = Synthetic Polypeptide
GGGGSGGGS

SEQ ID NO: 693 - protein, 10 aa, synthetic construct
  REGION 1..10: note = Synthetic Polypeptide
GGGGSGGGGS

SEQ ID NO: 694 - protein, 15 aa, synthetic construct
  REGION 1..15: note = Synthetic Polypeptide
GGGGSGGGGSGGGGS

SEQ ID NO: 695 - protein, 20 aa, synthetic construct
  REGION 1..20: note = Synthetic Polypeptide
GGGGSGGGGSGGGGSGGGGS

SEQ ID NO: 696 - protein, 25 aa, synthetic construct
  REGION 1..25: note = Synthetic Polypeptide
GGGGSGGGGSGGGGSGGGGSGGGGS

SEQ ID NO: 697 - protein, 30 aa, synthetic construct
  REGION 1..30: note = Synthetic Polypeptide
GGGGSGGGGSGGGGSGGGGSGGGGSGGGGS

SEQ ID NO: 698 - protein, 35 aa, synthetic construct
  REGION 1..35: note = Synthetic Polypeptide
GGGGSGGGGSGGGGSGGGGSGGGGSGGGGSGGGGS

SEQ ID NO: 699 - protein, 40 aa, synthetic construct
  REGION 1..40: note = Synthetic Polypeptide
GGGGSGGGGSGGGGSGGGGSGGGGSGGGGSGGGGSGGGGS

(19) United States
(12) Patent Application Publication
(10) Pub. No.: US 2025/0154255 A1
(43) Pub. Date: May 15, 2025

(54) DOSING REGIMEN FOR BCMAx-CD3 ANTIBODIES CONSTRUCTS

(71) Applicants: AMGEN RESEARCH (MUNICH) GMBH, Munich (DE); AMGEN INC., Thousand Oaks, CA (US)

(72) Inventors: Gerhard Zugmaier, Munich (DE); Max Topp, Würzburg (DE); Gerd Munzert, Munich (DE)

(21) Appl. No.: 19/022,400

(22) Filed: Jan. 15, 2025

Related U.S. Application Data
(63) Continuation of application No. 17/264,386, filed on Jan. 29, 2021.
(60) Provisional application No. 62/712,357, filed on Jul. 31, 2018.

(51) Int. Cl. C07K 16/28; A61K 39/00

(52) U.S. Cl. CPC C07K 16/2809; C07K 16/2878; A61K 2039/505; A61K 2039/545; C07K 2317/21; C07K 2317/31; C07K 2317/34; C07K 2317/94

(57) ABSTRACT

The present invention relates to an antibody construct comprising a first domain which binds to BCMA and a second domain which binds to CD3, for use in the treatment or amelioration of a BCMA positive neoplasm, wherein the antibody construct is administered at a specified dose in at least one cycle, wherein one cycle comprises a specified period of administration of the antibody construct. Moreover, the invention relates to a method for the treatment of a BCMA positive neoplasm comprising administering a specified amount of such antibody construct, and the use of such antibody construct for the manufacture of a medicament for the treatment of a BCMA positive neoplasm.

This application contains, as a separate part of disclosure, a Sequence Listing in computer-readable form (Filename: 53347B_SeqListing.xml; Size: 928,638 bytes; Created: Jan. 8, 2025) which is incorporated by reference herein in its entirety.

The present invention relates to an antibody construct comprising a first domain which binds to BCMA and a second domain which binds to CD3, for use in the treatment or amelioration of a BCMA positive neoplasm, wherein the antibody construct is administered at a specified dose in at least one cycle, wherein one cycle comprises a specified period of administration of the antibody construct. Moreover, the invention relates to a method for the treatment of a BCMA positive neoplasm comprising administering a specified amount of such antibody construct, and the use of such antibody construct for the manufacture of a medicament for the treatment of a BCMA positive neoplasm.

### BACKGROUND OF THE INVENTION

Multiple Myeloma (MM) is a malignant tumor of plasma cells which proliferate in bone marrow and release para protein. The resulting clinical laboratory pictures include infection, bone destruction, bone marrow failure, renal failure and hypercalcemia. The age adjusted annual incidence is increasing with approximately 6 new cases per 100,000. The incidence 2 times higher in the black US population than in Caucasians. The 5-year survival rate for MM has increased from ˜25% for newly diagnosed patients in 1975 to ˜45% in 2006. This improvement is mainly due to new drugs such as proteasome inhibitors and immunomodulators. However, MM is not considered curable with current approaches. Patients refractory to proteasome inhibitors and immunomodulators show an unfavorable outcome with a median overall survival of 9 months.

Outcome is particularly poor in high-risk populations such as the subgroup with del17p13 positive MM. Although many drugs are in clinical development for MM, new treatment options are still needed. Patients who show symptomatic disease are initially treated with primary induction therapy followed by high dose chemotherapy with autologous stem cell support in eligible patients. Patients eligible for intensive treatment are determined by age (65 to 75 years as upper limit), no comorbidities and intact renal function. Although this regimen has improved survival of younger and fit patients, the median duration of response does not exceed 3 years, and few patients remain free of the disease for more than 10 years.

Consolidation and maintenance approaches have been tested in order to increase the depth and duration of remission. Because maintenance treatment is challenging due to no efficacy or tolerability, there is still the option of improving survival outcome in the transplant setting by adding novel treatments to induction, consolidation or maintenance regimens. Patients not eligible for high dose therapy commonly receive induction regimens similar to the ones for the transplant candidates. These regimens include the proteasome inhibitor Bortezomib or a Melphalan based combination with Thalidomide. Median overall survival (OS) of Melphalan-Thalidomide-Prednisone (MPT) in elderly patients is 40 months. Lenalidomide in combination with Dexamethasone is the standard regimen for relapsed/refractory MM, but may move to the first line setting in transplant ineligible patients.

Other established regimens in the relapsed setting are repeat induction regimens or bortezomib or immunomodulator based salvage combinations with alkylating agents. An improvement of outcome (progression-free survival (PFS) and OS) is needed for patients in relapsed disease. Patients which are refractory to established treatments and progressing on treatment have a dismal outcome of 9 months OS on treatment and 3 months without treatment. The unmet need is highest in these patients.

Bispecific molecules such as BiTE® (bispecific T cell engager) antibody constructs are recombinant protein constructs having one binding domain that is specific for a selected tumor-associated surface antigen on target cells, and a second binding domain that is specific for CD3, a subunit of the T cell receptor complex on T cells. By their particular design, BiTE® antibody constructs are uniquely suited to transiently connect T cells with target cells and, at the same time, potently activate the inherent cytolytic potential of T cells against target cells. The first generation of BiTE® antibody constructs (see WO 99/54440 and WO 2005/040220) was brought into the clinic as AMG 103 (blinatumomab, anti-CD19 x anti-CD3) and AMG 110 (solitomab, anti-EpCAM x anti-CD3). Blinatumomab, which is approved for patients with ALL, is administered via continuous intravenous infusion, with a lower initial dose in the first period of administration and a higher dose in the remaining treatment, during the first cycle and in all subsequent cycles. In a comparison of step dosing versus constant (flat) dosing as a means to reach the target dose of blinatumomab, step dosing was found to be more effective at mitigating adverse events. In view of the positive efficacy signal and favorable safety profile, a similar administration scheme has hence been used for solitomab which was also administered with increasing doses in each cycle. An important further development of the first generation of BiTER antibody constructs was the provision of bispecific antibody constructs binding to a context independent epitope at the N-terminus of the CD3-epsilon chain of human and Callithrix jacchus, Saguinus oedipus or Saimiri sciureus (WO 2008/119567). The first BITE® molecule comprising this new CD3-epsilon binding domain that was tested in the clinic was AMG 330. In line with the previously used administration schemes, where the initiation of treatment with a lower dose before escalation to the target dose reduced the magnitude of cytokine elevation, AMG 330 was also administered with a step dosing regimen. The administration of a lower dose of an anti-target x anti-CD3 antibody construct in the first dose step is understood as a run-in phase or adaptation phase which should avoid or limit side effects resulting from the first contact of the patient with the antibody construct.

B cell maturation antigen (BCMA, TNFRSF17, CD269) is a transmembrane protein belonging to the TNF receptor super family. BCMA expression is selectively induced during late stage plasma cell differentiation and is absent on naïve and memory B cells. Upon BCMA binding to its ligands, B cell activating factor (BAFF) and a proliferation inducing ligand (APRIL), the survival of the bone marrow plasma cells and plasmablasts is promoted. BCMA does not maintain normal B cell homeostasis, but is required for the survival of long lived plasma cells. Studies in BCMA −/− mice showed that the survival of long lived bone marrow plasma cells was impaired, but B cell development and early humoral immune responses were indistinguishable from wild type mice. The mRNA expression of BCMA is highly elevated in malignant plasma cell disorders. By contrast, mRNA expression in normal tissues is very low and restricted to lymphoid tissues where normal long-lived plasma cells are located. BCMA protein expression is reported to be restricted to plasma cells only. Expression of BCMA is confined to plasma blasts and long-lived plasma cells and cannot be detected on other normal human tissues. BCMA is universally expressed on the cell surface of MM cells and at relatively higher levels on malignant plasma cells than the level observed on normal plasma cells. There is no correlation between BCMA expression and MM disease stage, response to last treatment and time from diagnosis. Neither T cells nor myeloid cells or CD34+ hematopoietic stem cells express BCMA. The selective expression of BCMA makes it a very attractive target for antibody-based and chimeric antigen receptor (CAR)-based therapies.

### DETAILED DESCRIPTION OF THE INVENTION

AMG 420 (formerly BI 836909) is a bispecific T cell engager which binds to BCMA on target cells as well as to CD3-epsilon on T cells. It functions as a bridge between MM cells and cytotoxic T lymphocytes (CTLs) by directing the cytolytic activity of CTLs to MM cells. AMG 420 consists of two single chain variable fragments (scFv), one being directed to BCMA and the other one to CD3. Each of the scFv fragments consists of a VH and a VL domain connected with a glycine/serine linker. The two scFv fragments are also connected with a glycine/serine linker.

The inventors surprisingly found that in the case of the anti-BCMA x anti-CD3 antibody construct, it is most beneficial for an MM patient that the target dose in the patient's serum is reached without delay. It is believed that the faster the target dose is achieved in the patient, the faster the onset of response. This can be demonstrated by the quick onset of CR in ALL, a tumor in bone marrow, which can be easily reached by the T cells (Stackelberg et al. JCO 2016). On the other hand, if it takes longer for the antibody construct to reach its therapeutically effective dose in a patient's body (e.g. due to step dosing), the antibody construct seems to be less efficient. Without wanting to be bound by the theory, the inventors believe that this phenomenon is related to T cell expansion and T cell redistribution.

It is hence an object of the present invention to provide an administration scheme for an anti- BCMA x anti-CD3 antibody construct which provides for a favorable safety and tolerability profile while resulting in a positive efficacy signal.

The present invention therefore relates to an antibody construct comprising a first domain which binds to BCMA and a second domain which binds to CD3, for use in the treatment or amelioration of a BCMA positive neoplasm, wherein the antibody construct is administered at a dose of 6.5 μg/day up to 650 μg/day in at least one cycle, wherein one cycle comprises a period of administration of the antibody construct of at least seven consecutive days.

Moreover, the invention relates to a method for the treatment or amelioration of a BCMA positive neoplasm, comprising administering an antibody construct comprising a first domain which binds to BCMA and a second domain which binds to CD3 to a subject in need thereof, wherein the antibody construct is administered at a dose of 6.5 μg/day up to 650 μg/day in at least one cycle, wherein one cycle comprises a period of administration of the antibody construct of at least seven consecutive days.

The invention further relates to the use of an antibody construct comprising a first domain which binds to BCMA and a second domain which binds to CD3 for the manufacture of a medicament for the treatment of a BCMA positive neoplasm, wherein the antibody construct is administered at a dose of 6.5 μg/day up to 650 μg/day in at least one cycle, wherein one cycle comprises a period of administration of the antibody construct of at least seven consecutive days.

The invention also relates to the use of an antibody construct comprising a first domain which binds to BCMA and a second domain which binds to CD3 for the treatment or amelioration of a BCMA positive neoplasm, wherein the antibody construct is administered at a dose of 6.5 μg/day up to 650 μg/day in at least one cycle, wherein one cycle comprises a period of administration of the antibody construct of at least seven consecutive days.

A “neoplasm” is an abnormal growth of tissue, usually but not always forming a mass. When also forming a mass, it is commonly referred to as a “tumor”. In brain tumors, the uncontrolled division of cells means that the mass of a neoplasm increases in size, and in a confined space such as the intracranial cavity this quickly becomes problematic because the mass invades the space of the brain pushing it aside, leading to compression of the brain tissue and increased intracranial pressure and destruction of parenchyma. According to the invention, the term “neoplasm” or “tumor” also refers to a condition that would benefit from treatment with the antibody construct as described herein. This includes chronic and acute disorders or diseases, including those pathological conditions that predispose a mammal to the condition (neoplasm or tumor) in question.

Neoplasms or tumors can be benign, potentially malignant (pre-cancerous), or malignant (cancerous). Malignant neoplasms/tumors are commonly called cancer. They usually invade and destroy the surrounding tissue and may form metastases, i.e., they spread to other parts, tissues or organs of the body. A “primary tumor” is a tumor growing at the anatomical site where tumor progression began and proceeded to yield a cancerous mass. For example, a brain tumor occurs when abnormal cells form within the brain. Most cancers develop at their primary site but then go on to form metastases or spread to other parts (e.g. tissues and organs) of the body. These further tumors are secondary tumors. Most cancers continue to be called after their primary site, even after they have spread to other parts of the body.

Lymphomas and leukemias are hematopoietic or lymphoid neoplasms. For the purposes of the present invention, lymphomas and leukemias are also encompassed by the terms “tumor”, “cancer” or “neoplasm”. Lymphoma is a group of blood cancers that develop from lymphocytes (a type of white blood cell). Leukemia is a group of cancers that usually begin in the bone marrow and result in high numbers of abnormal white blood cells. These white blood cells are not fully developed and are called blasts or leukemia cells. Lymphomas and leukemias are a part of the broader group of tumors of the hematopoietic and lymphoid tissues.

For the purposes of the present invention, the terms “neoplasm”, “tumor” and “cancer” may be used interchangeably, and they comprise both primary tumors/cancers and secondary tumors/cancers (or “metastases”), as well as mass-forming neoplasms (tumors) and lymphoid neoplasms (such as lymphomas and leukemias), and also MRD.

The term “minimal residual disease” (MRD) refers to the evidence for the presence of small numbers of residual cancer cells that remain in the patient after cancer treatment, e.g. when the patient is in remission (the patient has no symptoms or signs of disease). A very small number of remaining cancer cells usually cannot be detected by routine means because the standard tests used to assess or detect cancer are not sensitive enough to detect MRD. Nowadays, very sensitive molecular biology tests for MRD are available, such as flow cytometry, PCR and next-generation sequencing. These tests can measure minimal levels of cancer cells in tissue samples, sometimes as low as one cancer cell in a million normal cells. In the context of the present invention, the terms “prevention”, “treatment” or “amelioration” of a neoplasm are envisaged to also encompass “prevention, treatment or amelioration of MRD”, whether the MRD was detected or not.

Is it envisaged that the BCMA positive neoplasm is a B cell neoplasm or a plasma cell neoplasm. B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells) and secrete cytokines. In mammals, B cells mature in the bone marrow, which is at the core of most bones. B cells, unlike the other two classes of lymphocytes—T cells and natural killer (NK) cells—express B cell receptors (BCRs) on their cell membrane. BCRs allow the B cell to bind to a specific antigen, against which it will initiate an antibody response. Plasma cells, also called plasma B cells, plasmocytes, or effector B cells, are white blood cells that secrete large volumes of antibodies. They are usually transported by the blood plasma and the lymphatic system. Plasma cells originate in the bone marrow. B cells differentiate into plasma cells that produce antibody molecules closely modelled after the receptors of the precursor B cell. Once released into the blood and lymph, these antibody molecules bind to the target antigen and initiate its neutralization or destruction.

The level of expression of BCMA on the cell surface can be determined e.g. by flow cytometry analysis. The subpopulation of cells (e.g. B cells, plasma cells, MM cells, CD138+ cells) that is selected for analysis of BCMA expression can e.g. be stained with an anti-BCMA antibody, followed by a secondary antibody, and then analyzed in a FACS assay. A BCMA negative cell line (such as K562, A549, TC71, CCRF-CEM) can be used as control. A shift in the FACS assay (with the BCMA negative cell line defining 0% BCMA expression) indicates that the cells to be analyzed are BCMA positive. Different levels of BCMA expression can exist on the surface cells, such as low, medium or high expression. See also Quinn et al., Blood (2011) 117:890-901 and Sanchez et al, Br J Heamatol 2012 Jul. 18.

The “BCMA positive neoplasm” or the “(BCMA positive) B cell neoplasm or plasma cell neoplasm” can be selected from the group including, but not limited to, multiple myeloma, relapsed and/or refractory multiple myeloma, heavy chain multiple myeloma, light chain multiple myeloma, extramedullary myeloma (extramedullary plasmacytoma, extramedullary multiple myeloma), plasmacytoma, plasma cell leukemia, Waldenström's macroglobulinemia (lymphoplasmacytic lymphoma), and smoldering myeloma (smoldering multiple myeloma). The present disclosure hence also relates to an antibody construct for use in the treatment or amelioration of multiple myeloma (MM), plasmacytoma, plasma cell leukemia and Waldenström's macroglobulinemia, as described herein. The MM can be selected from the group consisting of or comprising relapsed and/or refractory multiple myeloma, heavy chain multiple myeloma, light chain multiple myeloma, extramedullary multiple myeloma, and smoldering multiple myeloma.

The antibody construct of the invention (and the pharmaceutical composition comprising such antibody construct) is/are useful in the treatment, amelioration and/or prevention of the BCMA positive neoplasm as described herein in a subject in need thereof. The term “treatment” refers to both therapeutic treatment and prophylactic or preventative measures. Treatment includes the administration of the antibody construct (or the pharmaceutical composition comprising such antibody construct) to the patient's body, to an isolated tissue, or to a cell from a patient or a subject in need who has a BCMA positive neoplasm as described herein, a symptom of such neoplasm, or a predisposition toward such neoplasm, with the purpose to cure, heal, alleviate, relieve, alter, remedy, ameliorate, improve, or affect the BCMA positive neoplasm, one or more symptoms of the BCMA positive neoplasm, or the predisposition toward the disease.

The terms “subject in need”, “patient” or those “in need of treatment” include those already with the BCMA positive neoplasm, as well as those in an MRD setting and those in which the neoplasm is to be prevented. The terms also include human and other mammalian subjects that receive either prophylactic or therapeutic treatment.

The term “amelioration” as used herein refers to any improvement of the disease state (the disease being a BCMA positive neoplasm) of a patient, by the administration of an antibody construct according to the invention to such patient or subject in need thereof. Such an improvement may be seen as a slowing down the progression or stopping the progression of the disease of the patient, and/or as a decrease in severity of disease symptoms, an increase in frequency or duration of disease symptom-free periods or a prevention of impairment or disability due to the disease.

The term “prevention” as used herein means the avoidance of the occurrence or of the re- occurrence of a disease as specified herein, by the administration of an antibody construct according to the invention to a subject in need thereof.

In the case of Multiple Myeloma, the symptoms and signs vary greatly because many organs can be affected by the disease. The common symptoms of multiple myeloma include elevated calcium levels, renal failure, anemia, and bone lesions (together, “CRAB” features). In advanced MM, bone pain, bleeding, and frequent infections may occur. Complications may also include amyloidosis. The International Myeloma Working Group (IMWG) has established criteria for the diagnosis of MM which teach, in addition to the classic CRAB features, three “myeloma defining events” (MDEs):
60% or greater clonal plasma cells on bone marrow examination
Serum involved/uninvolved free light chain ratio of 100 or greater, provided the absolute level of the involved light chain is at least 100 mg/L (a patient's “involved” free light chain—either kappa or lambda—is the one that is above the normal reference range; the “uninvolved” free light chain is the one that is typically in, or below, the normal range).
More than one focal lesion on MRI that is at least 5 mm or greater in size

The presence of at least one of these markers is considered sufficient for a diagnosis of multiple myeloma, regardless of the presence or absence of symptoms or CRAB features. See also Palumbo A. J Clin Oncol. 2014 Feb. 20; 32(6): 587-600.

Bone pain affects almost 70% of patients and is the most common symptom. It usually involves the spine and ribs. Involvement of the vertebrae may lead to spinal cord compression or kyphosis. Myeloma bone disease is due to the overexpression of receptor activator for nuclear factor κB ligand (RANKL) by bone marrow stroma. RANKL activates osteoclasts, which resorb bone. The resultant bone lesions are lytic (i.e. they cause breakdown) in nature, and are best seen in plain radiographs. The breakdown of bone also leads to the release of calcium into the blood, leading to hypercalcemia and its associated symptoms.

The anemia found in myeloma is usually normocytic and normochromic. It results from the replacement of normal bone marrow by infiltrating tumor cells and inhibition of normal red blood cell production (hematopoiesis) by cytokines.

A bone marrow biopsy can be performed to estimate the percentage of bone marrow occupied by plasma cells. This percentage is used in the diagnostic criteria for MM. Usually, MM patients have ≥10% clonal bone marrow plasma cells. Immunohistochemistry (staining particular cell types using antibodies against surface proteins) can detect plasma cells which express immunoglobulin in the cytoplasm and occasionally on the cell surface; for example, myeloma cells are typically positive for the markers CD56, CD38, CD138, CD319, but other markers may be included as well to define or identify MM.

The so-called “paraprotein” (also called myeloma protein, monoclonal protein or M protein,) is an abnormal immunoglobulin fragment that is produced in excess by an abnormal monoclonal proliferation of plasma cells, typically in MM. In theory, MM patients can produce all classes of immunoglobulin, but IgG paraproteins are most common, followed by IgA and IgM, while IgD and IgE myeloma are very rare. In addition, antibody light chains and/or heavy chains may be secreted in isolation: kappa or lambda light chains or any of the five types of heavy chains (alpha, gamma, delta, epsilon or my (μ)-heavy chains). This proliferation of the paraprotein has several deleterious effects on the body, including impaired immune function, abnormally high blood viscosity, and kidney damage. Patients without evidence of paraprotein may have “nonsecretory” myeloma (not producing immunoglobulins); they represent approximately 3% of all MM patients. The presence of serum and/or urinary paraprotein is an indicator for MM, except in patients with true nonsecretory MM. Quantitative measurements of the paraprotein in urine and/or serum of a patient can be used to establish a diagnosis and/or to monitor the disease.

Kidney failure may develop both acutely and chronically. The most common cause of kidney failure in MM is due to proteins secreted by the malignant cells. Myeloma cells produce monoclonal proteins of varying types, most commonly immunoglobulins (antibodies) and free light chains, resulting in abnormally high levels of these proteins (paraproteins) in the blood. Depending on the size of these proteins, they may be excreted through the kidneys, but kidneys can also be damaged by their effects. Furthermore, increased bone resorption leads to hypercalcemia and causes nephrocalcinosis, thereby contributing to the kidney failure.

The most common infections occurring in MM are pneumonias and pyelonephritis. The increased risk of infection is due to immune deficiency. Although the total immunoglobulin level is typically elevated in MM, the majority of the antibodies are ineffective monoclonal antibodies from the clonal plasma cell.

It is envisaged that the administration of the antibody construct in the treatment or amelioration of a BCMA positive neoplasm according to the invention.
reduces the level of paraprotein or free light chain in the urine and/or serum by at least about 20%, at least about 30%, at least about 40%, at least about 50%, at least about 60%, at least about 70%, at least about 80%, or at least about 90% relative to the paraprotein or free light chain level in the urine and/or serum, respectively, prior to the start of the treatment, i.e. prior to the first administration of the antibody construct (“prior to”, in this specific context, means within 1, 2, 4, 6, 8, or 12 hours before, within 1, 2, 3, 4, 5 or 6 days before, within 1, 2, 3 or 4 weeks before, or within 1, 2, 3 or 4 months before);
reduces the percentage of plasma cells in the bone marrow by at least about 20%, at least about 30%, at least about 40%, at least about 50%, at least about 60%, at least about 70%, at least about 80%, or at least about 90% relative to the percentage of plasma cells in the bone marrow prior to the start of the treatment, i.e. prior to the first administration of the antibody construct (“prior to”, in this specific context, means within 1, 2, 4, 6, 8, or 12 hours before, within 1, 2, 3, 4, 5 or 6 days before, within 1, 2, 3 or 4 weeks before, or within 1, 2, 3 or 4 months before);
induces a reduction of any of the symptoms described above by at least about 20%, at least about 30%, at least about 40%, at least about 50%, at least about 60%, at least about 70%, at least about 80%, or at least about 90% relative to the symptoms prior to the start of the treatment, i.e. to the first administration of the antibody construct (“prior to”, in this specific context, means within 1, 2, 4, 6, 8, or 12 hours before, within 1, 2, 3, 4, 5 or 6 days before, within 1, 2, 3 or 4 weeks before, or within 1, 2, 3 or 4 months before, depending on the symptom);
inhibits tumor growth or tumor cell proliferation by at least about 20%, at least about 30%, at least about 40%, at least about 50%, at least about 60%, at least about 70%, at least about 80%, or at least about 90% relative to untreated patients or relative to untreated cells; and/or.
induces lysis of the cells of the BCMA positive neoplasm of at least about 20%, at least about 30%, at least about 40%, at least about 50%, at least about 60%, at least about 70%, at least about 80%, or at least about 90% relative to untreated patients or relative to untreated cells.

The ability of an antibody construct of the invention to inhibit tumor growth/tumor cell proliferation or to induce cell lysis may be evaluated in an animal model predictive of efficacy in human tumors, or in an in vitro or ex vivo study (such as depletion of BCMA positive cells by autologous T cells from a multiple myeloma patient's BM aspirate induced by the antibody construct). Efficacy assessments of the antibody construct may furthermore be performed as follows: Tumor assessment can be done by analysis of percent myeloma involvement, by FISH (fluorescent in situ hybridization) as well as by karyotyping in the bone marrow (BM). Data for BM karyotyping and FISH may be obtained from a BM sample. Serum protein electrophoresis (SPEP) and urine protein electrophoresis (UPEP) allow for measurement of serum/urine M protein. Immunofixation is another means to detect serum and/or urine M protein. It is also envisaged that serum free light chain assay and ratio analysis can be performed. In case of free light chain (FLC) multiple myeloma, FLC can be analyzed in serum and urine (sFLC and uFLC). Levels of involved/uninvolved FLC, ratio of monoclonal lambda-FLC/kappa-FLC, and ratio of monoclonal kappa-FLC/lambda-FLC can be determined. Furthermore, quantitative and qualitative immunoglobulin (Ig) can also be analyzed, and beta-2 microglobulin in serum can be assessed. It is also envisaged that skeletal survey and plasmacytoma assessments can be performed. Screening imaging to evaluate for extramedullary relapse using whole-body MRI or PET/CT can be performed. Imaging appropriate for assessment of bone lesions includes, but is not limited to, CT scan, MRI, PET, PET-CT, or other standard-of-care method. It is also envisaged that minimal residual disease is measured by a next generation sequencing (NGS) based assay. For this purpose, bone marrow aspirates can be collected from subjects suspected to be complete responders. Plasma samples can additionally be collected from subjects at the same time points as BM MRD samples are collected, to assess the feasibility of MRD detection on ctDNA (circulating tumor DNA). MRD response may be defined as <1 tumor cell/10⁴ normal cells in the bone marrow per FACS using antibodies to cytlgλ, cytlgκ, CD19, CD56 or CD138, CD38, and CD45, as needed. In one embodiment of the invention, these markers are a sufficient condition to define a tumor cell in the context of the present invention.

It is envisaged that a patient's or subject's response to the administration of the antibody construct according to the invention is measured in one of the following ways:
quantitative measurement of the paraprotein (M protein) or free light chain in the urine and/or serum;
determination of the percentage of plasma cells in the bone marrow; and/or
imaging (CT, MRI) of extramedullary manifestations

The antibody construct of the invention is administered for at least one cycle, but more cycles of administration such as 2, 3, 4, 5, 6, 7, 8, 9 or 10 cycles are also envisaged. For example, an administration of 1-5 cycles is considered to be beneficial for a patient in need.

According to the invention, “one cycle” comprises a period of administration of the antibody construct of at least seven consecutive days (one week). It is furthermore envisaged that one cycle comprises a period of administration of the antibody construct, followed by a period without administration of the antibody construct (“treatment holiday”, “pause” or “break”). If a patient receives only one single cycle, this cycle ends with the last day of administration of the antibody construct. On the other hand, if a patient receives more than one cycle, each cycle comprises a period of administration followed by a period without administration. The period without administration of the antibody construct (“pause”) is defined by its length, see below. It is envisaged that if a patient receives more than one cycle (e.g. 2, 3, 4, 5, 6, 7, 8, 9 or 10 cycles), the last administration cycle does not comprise a period without administration of the antibody construct (i.e. the last cycle ends with the last day of administration).

The period of administration of the antibody construct is at least seven consecutive days (one week). It is envisaged that this period is up to eight weeks. Any duration between one week and eight weeks is also envisaged, such as two weeks, three weeks, four weeks, five weeks, six weeks, or seven weeks, or any other period between one week and eight weeks composed of a certain number of weeks and a certain number of days (such as, one, two, three, four, five, or six days). The period of administration can hence also be 7, 10, 14, 15, 20, 21, 25, 28, 30, 35, 40, 42, 45, 48 50, 55, or 56 consecutive days. The period of administration can e.g. be from 1 to 8 weeks, from 1 to 7 weeks, from 2 to 6 weeks, from 3 to 5 weeks, or from 25 to 30 days. For example, four weeks of administration (i.e. 28 consecutive days) within one cycle is considered to be beneficial for a patient in need. Independent of its specific duration, it is envisaged that the period of administration is characterized by a daily administration of the antibody construct.

According to one embodiment of the invention, the period without administration of the antibody construct is at least seven consecutive days (one week), and it may be up to three months. Any duration between one week and three months is also envisaged, such as two weeks, three weeks, four weeks, one month, five weeks, six weeks, seven weeks, eight weeks, two months, nine weeks, ten weeks, eleven weeks, twelve weeks or thirteen weeks, or any other period between one week and three months composed of a certain number of months and/or a certain number of weeks and/or a certain number of days (such as, one, two, three, four, five, or six days). The period without administration of the antibody construct can e.g. be from 1 week to 3 months, from 1 week to 2 months, from 1 week to 1 month, from 1 week to 4 weeks, from 1 week to 3 weeks, from 10 days to 20 days, or from 10 to 15 days. For example, two or three weeks without administration within one cycle is considered to be particularly beneficial for a patient in need. Starting after the fourth cycle, a longer period without administration of the antibody construct may be implemented, such as a period of three to five weeks, or four weeks.

According to one embodiment, the antibody construct is administered in one to five cycles, preferably four or five cycles, wherein one cycle comprises or consists of a period of administration of two to five weeks (i.e. 14 to 35 consecutive days), preferably four weeks (i.e. 28 consecutive days), followed by a period without administration of one to four weeks, preferably two or three weeks.

The different administration cycles that a patient can receive do not have to be fully identical in terms of the length of the period of administration of the antibody construct and the length of the period without its administration. As a consequence, the entire length of the different cycles is not necessarily identical either. For example, the length of the period of administration can vary between the different cycles; it can e.g. become longer or shorter with every cycle or from one cycle to the next. Alternatively or additionally, the length of the period without administration can vary between the different cycles; it can e.g. become longer or shorter with every cycle or from one cycle to the next. The same applies to the dose of the antibody construct, as discussed herein below.

According to the invention, the antibody construct is administered at a dose of 6.5 μg/day up to 650 μg/day in at least one cycle, wherein one cycle comprises a period of administration of at least seven consecutive days (one week). This means that the antibody construct is administered for at least 7 consecutive days at a dose between and including 6.5 μg/day and 650 μg/day. The dose of the antibody construct may be 6.5, 10, 20, 30, 40, 50, 60, 70, 80, 90, 100, 125, 150, 200, 250, 300, 350, 400, 450, 500, 550, 600 or 650 μg/day. According to one embodiment, the dose is about 400 μg/day. A preferred dose or dose range of the antibody construct of the invention may also be 6.5 to 650 μg/day, 10 to 600 μg/day, 20 to 600 μg/day, 30 to 600 μg/day, 40 to 600 μg/day, 50 to 600 μg/day, 60 to 600 μg/day, 70 to 600 μg/day, 80 to 600 μg/day, 90 to 600 μg/day, 100 to 600 μg/day, 150 to 650 μg/day, 200 to 600 μg/day, 300 to 600 μg/day, 250 to 550 μg/day, 300 to 550 μg/day, 300 to 500 μg/day, 350 to 450 μg/day, 350 to 650 μg/day, or 400 to 600 μg/day. It is envisaged that these doses may apply for an antibody construct having a molecular weight of about 20 to about 90 kDa, about 30 to about 80 kDa, about 40 to about 70 kDa, about 50 to about 60 kDa, about 52 to about 58 kDa, and preferably about 54 to about 56 kDa. In case the antibody construct has a different (lower or higher) molecular weight, respective equimolar doses can easily be determined. For example, assuming that one preferred antibody construct according to the invention has a molecular weight of 55 kDa, then 650 μg of this antibody construct correspond to 1.18 x 10⁻⁸ mol. Likewise, 6.5 μg of this antibody construct correspond to 1.18 x 10⁻¹⁰ mol.

It is envisaged that the antibody construct of the invention has a molecular weight of about 20 to about 90 kDa, about 30 to about 80 kDa, about 40 to about 70 kDa, about 50 to about 60 kDa, about 52 to about 58 kDa, and preferably about 54 to about 56 kDa. It is furthermore envisaged that the antibody construct of the invention has a terminal elimination half-life or an elimination half-life (T_{1/2}) of about 3-36 h, about 6-30 h, or about 12-24 h. “Half-life” is the time required for a quantity to reduce to half its initial value. The medical sciences refer to the half-life of substances or drugs in the human body. In a medical context, half-life may refer to the time it takes for a substance/drug to lose one-half of its activity, e.g. pharmacologic, physiologic, or radiological activity. The half-life may also describe the time that it takes for the concentration of a drug or substance in blood plasma to reach one-half of its steady-state value (“elimination half-life”). Typically, the elimination or removal of an administered substance/drug refers to the body's cleansing through biological processes such as metabolism, excretion, also involving the function of kidneys and liver. The “first-pass metabolism” is a phenomenon of drug metabolism whereby the concentration of a drug is reduced before it reaches the circulation. It is the fraction of drug lost during the process of absorption. Accordingly, by “hepatic first-pass metabolism” is meant the propensity of a drug to be metabolized upon first contact with the liver, i.e. during its first pass through the liver.

It is envisaged that the antibody construct is administered at a dose of 6.5 μg/day up to 650 μg/day in at least one cycle, wherein one cycle comprises a period of administration of at least seven consecutive days (one week), and wherein the dose is constant during each cycle. A “constant” dose means that during the period of administration within one cycle, the same dose is administered to the patient every day. It is furthermore envisaged that the antibody construct is administered at a dose of 6.5 μg/day up to 650 μg/day in more than one cycle (e.g. from 1 up to 10 cycles), wherein one cycle comprises a period of administration of at least seven consecutive days (one week), and wherein the dose is constant during each cycle and from one cycle to the subsequent cycle. This could mean e.g. that the antibody construct is administered at the same dose in cycles 1 and 2, in cycles 2 and 3, in cycles 3 and 4, in cycles 4 and 5, in cycles 1 to 3, in cycles 2 to 4, in cycles 3 to 5, in cycles 1 to 4, in cycles 2 to 5, or in cycles 1 to 5, etc. It is furthermore envisaged that the antibody construct is administered at a dose of 6.5 μg/day up to 650 μg/day in more than one cycle (e.g. from 1 up to 10 cycles), wherein one cycle comprises a period of administration of at least seven consecutive days (one week), and wherein the dose is constant during each cycle, but differs between the cycles. For example, the dose of the antibody construct can increase from one cycle to the subsequent cycle, or can decrease from one cycle to the subsequent cycle. The antibody construct might e.g. be administered at a dose of 400 μg/day during one or more cycles (such as the first cycle or the first and second cycle), and at a dose of 500 μg/day or 600 μg/day during one or more subsequent cycles (such as the second, third, fourth or fifth cycle). It is also envisaged that the antibody construct is administered at a dose of 6.5 μg/day up to 650 μg/day in at least one cycle, wherein one cycle comprises a period of administration of at least seven consecutive days (one week), and wherein the dose increases during at least one cycle. The dose increase may be during the first cycle or during the first and the second cycle, but may also be during more than the first two cycles. The lower dose may be administered during a period of 1-7 days, 1-5 days, 2-4 days or 3 days, followed by a higher dose administered until the end of the cycle. As an example, the lower dose may be 100-300 μg/day or 200 μg/day, followed by a higher dose of 350-450 μg/day or 400 μg/day; or the lower dose may e.g. be 300-500 μg/day or 400 μg/day, followed by a higher dose of 550-650 μg/day or 600 μg/day; or the lower dose may be 200 μg/day, for example during a period of 3 days, for example during the first cycle, followed by a higher dose of 400 or 600 or 650 μg/day administered until the end of the first cycle. In the latter case, it is envisaged that from the second cycle onwards, the respective higher dose is administered. It is envisaged that the higher dose does not exceed 650 μg/day.

The antibody construct of the invention will generally be designed for specific routes and methods of administration. A route of administration in pharmacology is the path by which a substance is taken into the body. Routes of administration are generally classified by the location at which the substance is applied, these can be topical (local), enteral (system-wide effect of the substance, but delivered through the gastrointestinal tract), or parenteral (systemic action of the substance, but delivered by routes other than the gastrointestinal tract). In the context of the present invention, the routes of administration include topical, enteral and parenteral routes. The reason for the choice of routes of drug administration are governed by various factors such as:
Physical and chemical properties of the drug. The physical properties are solid, liquid and gas. The chemical properties are solubility, stability, pH, irritancy etc.
Site of desired action: The action may be localized and approachable or generalized and not approachable.
Rate of extent of absorption of the drug from different routes.
Condition of the patient.

The antibody construct of this invention (and a pharmaceutical composition comprising this antibody construct) is particularly useful for parenteral administration. Parenteral administration generally acts more rapidly than topical or enteral administration, and often comes along with a very high bioavailability of up to 100% (in particular, in the case of IV administration). In general, parenteral administration includes, but is not limited to, intravenous, intracerebral, intraarterial, intraperitoneal, intraosseous, and intravesical delivery.

The administration according to the present invention is preferably intravenous. Parenteral or intravenous administration can be performed by injection (e.g. using a needle and a syringe) or by infusion (e.g. via a catheter and a pump system). It is hence envisaged that the administration according to the present invention is via intravenous injection or via intravenous infusion. Usually, an IV infusion is administered via a line, a port or a catheter (small, flexible tube), such as a central venous access or a central venous catheter (CVC) which is a catheter placed into a large vein, or a peripheral venous catheter (PVC), which is a catheter placed into a peripheral vein. In general, catheters or lines can be placed in veins in the neck (internal jugular vein), chest (subclavian vein or axillary vein), groin (femoral vein), or through veins in the arms (also known as a PICC line, or peripherally inserted central catheters). Central IV lines have their catheters that are advanced through a vein and empty into a large central vein, usually the superior vena cava, inferior vena cava or even the right atrium of the heart. A peripheral intravenous (PIV) line is used on peripheral veins (the veins in the arms, hands, legs and feet). A port is a central venous line that does not have an external connector; instead, it has a small reservoir that is covered with silicone rubber and is implanted under the skin. Medication is administered intermittently by placing a small needle through the skin, piercing the silicone, into the reservoir. When the needle is withdrawn, the reservoir cover reseals itself. The cover can accept hundreds of needle sticks during its lifetime.

The present invention provides for a bolus administration of the antibody construct of the invention. A bolus is the administration of a discrete amount of a medication, drug, or other compound within a specific negligible time, generally within 1-30 minutes. In most cases, the bolus administration is given intravenously. A bolus is usually administered via injection (e.g. an intravenous bolus injection), but a bolus infusion (e.g. an intravenous bolus infusion) is also possible. A short infusion is an infusion (usually an IV infusion), of a small volume (such as 20 mL to 100 mL), which is administered over a period of, at most, three hours, usually of 30 to 60 minutes.

Intravenous “intermittent infusion” is an infusion of a volume of medication over a set period of time, such as 20-120 minutes or 30-60 minutes, at prescribed intervals, such as every 4, 6, 8, 10, 12, 14, 16, 18, 20, 22 or 24 hours. The purpose is to administer small amounts of medication at regular intervals. An intermittent medication—like any other form of infusion—may be administered by gravity or via an electronic infusion device (EID), also known as an infusion pump.

In the case of an infusion, an infusion pump may be used to infuse the medication (antibody construct) into a patient's circulatory system. The pump is generally used intravenously, although arterial and epidural infusions with pumps are also possible. The solution for infusion may be prepared in bags for IV infusion and delivered through infusion lines. Infusion pumps can administer fluids in ways that would be unreliable if performed manually. For example, they can administer as little as 0.1 mL per hour injections, injections every minute, injections with repeated boluses, up to a maximum number per hour, or fluids whose volumes vary by the time of day. It is also possible that infusions are administered using only the pressure supplied by gravity. Different types of infusions according to the present invention include, but are not limited to, continuous infusion, bolus infusion, short infusion, and intermittent infusion. A continuous intravenous (cIV) administration or infusion is one preferred embodiment of the administration of the antibody construct according to the invention. It is for example envisaged that the antibody construct is administered as a cIV infusion at a constant flow rate.

The present invention provides for a continuous administration, i.e. an uninterrupted or substantially uninterrupted administration of the antibody construct/composition comprising the antibody construct of the invention. As a non-limiting example, this may be realized by a small pump system worn by the patient for metering the influx of the therapeutic agent into the body of the patient. The antibody construct of the invention (or a pharmaceutical composition comprising the antibody construct) can be administered using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of the therapeutic agent into the body of the patient may ensue. In such a case, the phase of administration prior to cartridge replacement and the phase of administration following cartridge replacement would still be considered within the meaning of the pharmaceutical means and methods of the invention and would together make up one “uninterrupted administration” of such therapeutic agent. The same applies for bag changes, i.e. in cases the continuous administration is realized by means of a bag (instead of a small cartridges) containing the antibody construct solution, or any other recipient or reservoir comprising the antibody construct of the invention for continuous administration. It is envisaged that the antibody construct of the invention is provided in infusion bags. These bags are changed in accordance with country regulations and local pharmacy standards for infusion of compounded sterile products, usually up to 48 hours in the US and up to 96 hours in Australia and Europe.

A continuous or uninterrupted administration of the antibody construct of the invention may be carried out by way of a fluid delivery device or small pump system including a fluid driving mechanism for driving fluid out of a reservoir and an actuating mechanism for actuating the driving mechanism. Pump systems for such administration may include a needle or a cannula for penetrating the skin of a patient and delivering the suitable composition into the patient's body. The pump system can be attached to the skin of the patient for 24 hours up to several days. The pump system may be of small size with a reservoir for small volumes. As a non-limiting example, the volume of the reservoir for the suitable pharmaceutical composition to be administered can be between 0.1 and 50 ml.

The (daily) dose of the antibody construct may e.g. be administered as a bolus administration (bolus injection or bolus infusion), an injection, a continuous administration, a continuous infusion, or as a short infusion. A continuous administration is preferred, and a continuous IV (cIV) administration/continuous IV infusion is most preferred.

Pharmaceutical compositions may be administered using a medical device. Examples of medical devices for administering pharmaceutical compositions are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

In the context of the present invention, it is envisaged that a premedication is administered prior to the start of administration of the antibody construct in the first cycle, and optionally also prior to the start of administration of the antibody construct in one or more of the following cycles, such as the second, third, fourth and/or fifth cycle. It is envisaged that “prior to”, in this specific context, means within 24 hours, 18 hours, twelve hours, six hours, five hours, four hours, or three hours, and preferably within 120, 90, 60 or 30 minutes before the start of administration of the antibody construct. The premedication may e.g. be administered 30-120 or 30-60 minutes prior to start of administration of the antibody construct. It is also envisaged that a comedication is administered concurrent with or after the start of administration of the antibody construct in the first cycle, and optionally also concurrent with or after the start of administration of the antibody construct in one or more of the following cycles, such as the second, third, fourth and/or fifth cycle. It is envisaged that “after”, in this specific context, means within 24 hours, 18 hours, twelve hours, six hours, five hours, four hours, or three hours, and preferably within 120, 90, 60, 30, 20, 15 or 10 minutes after the start of administration of the antibody construct. The comedication may e.g. be administered 10-120, 10-60, 10-30 or 15-20 minutes after start of administration of the antibody construct. The purpose of the premedication or comedication may be e.g. to prevent or reduce severity of infusion-related reactions. Premedication is preferred over comedication.

The premedication or comedication may include any one or a combination of the following:
Paracetamol (acetaminophen, APAP) or an equivalent; to be preferably administered orally (p.o.) or intravenously; and to be preferably administered at a dose of 100-4000 mg, preferably 200-3000 mg or 300-2500 mg or 400-2000 mg or 500-1500 mg, preferably 600-1400 mg, 700-1300 mg, 800-1200 mg, 900-1100 mg or about 1000 mg p.o. paracetamol (or an equivalent dose for an equivalent medication and/or another route of administration). The skilled person knows how to identify paracetamol equivalents. They include, but are not limited to, ibuprofen (to be administered e.g. at a dose of 100-3200 mg, preferably 200-3000 mg or 300-2500 mg or 400-2000 mg, preferably 500-1500 mg, 600-1200 mg, 700-1000 mg, 750-900 mg or about 800 mg) and metamizole (to be administered e.g. at a dose of 100-4000 mg, preferably 200-3000 mg or 300-2500 mg or 400-2000 mg, or about 500-1000 mg)
One or more analgesics selected from meperidine, dipyrone, hydromorphone, fentanyl, and tramadol
Antihistamine, to be preferably administered orally or intravenously, and to be preferably administered at a dose equivalent to diphenhydramine 50 mg i.v. The skilled person knows how to identify antihistamines. They include, but are not limited to, antihistamines of oral, parenteral or rectal route such as: azatadine (maximum dose e.g. 4 mg/day), brompheniramine (maximum dose e.g. 30 mg/day), cetirizine (maximum dose e.g. 15 mg/day), chlorpheniramine (maximum dose e.g. 30 mg/day), clemastine (maximum dose e.g. 10 mg/day), cyproheptadine (maximum dose e.g. 15 mg/day), desloratadine (maximum dose e.g. 7 mg/day), dexchlorpheniramine (maximum dose e.g. 15 mg/day), diphenhydramine (maximum dose e.g. 350/per day), doxylamine (maximum dose e.g. 180 mg/day), fexofenadine (maximum dose e.g. 200 mg/day), loratadine (maximum dose e.g.15 mg/day), phenindamine (maximum dose e.g. 180 mg/day)
Glucocorticoid, to be preferably administered orally or intravenously, and to be preferably administered at a dose equivalent to 4-20 mg or 6-18 mg or 8-16 mg or 16 mg dexamethasone i.v. (the equivalence referring to the glucocorticoid potency)

All four substances or substance groups listed above may be administered as premedication or comedication, or a combination of only two or of three substances, or only one of the four substances. It is envisaged that the glucocorticoid (GC) dose administered before the start of the second cycle may be identical to the GC dose administered before start of the first cycle, or may be reduced to about 50% of the dose administered before start of the first cycle, or may be omitted for the second (and potentially any subsequent) cycle. A reduction of the GC dosage may apply e.g. if the antibody construct according to the invention is well tolerated without significant signs of infusion-related reactions during the first cycle. It is furthermore envisaged that the dose may further be reduced before start of the third and any subsequent cycle. Alternatively, while GC is administered as premedication (and potentially comedication) before the start of the first cycle, no GC premedication or comedication is administered in the second, third, fourth and/or fifth cycle. In general, the dose of the premedication or comedication that is to be used in accordance with the embodiments of the present invention will depend on the circumstances of the individual patient.

Glucocortocoids are a class of corticosteroids, which are a class of steroid hormones. Glucocorticoids are corticosteroids that bind to the glucocorticoid receptor. A less common synonym is glucocorticosteroid. Cortisol (known as hydrocortisone when used as a medication) is the most important human glucocorticoid. A variety of synthetic glucocorticoids, some far more potent than cortisol, have been created for therapeutic use. They differ in both pharmacokinetics (e.g. absorption factor, half-life, volume of distribution, clearance) and pharmacodynamics (e.g. glucocorticoid potency or mineralocorticoid potency). Cortisol is the standard of comparison for glucocorticoid potency. One example for commonly prescribed replacement steroid equivalents may be prednisone (5 mg)=cortisone (25 mg)=dexamethasone (0.75 mg)=hydrocortisone (20 mg)=methylprednisolone (4 mg). These doses indicate the equivalent pharmacologic dose of systemic glucocorticoids. Another corticosteroid comparison chart indicating the half-lives of the different substances can be found e.g. in www.nadf.us/downloads/adrenalhormone.pdf.

Examples of GCs to be used as premedication or comedication in the present embodiment include, but are not limited to, cortisone, hydrocortisone, prednisone, prednisolone, methylprednisolone, dexamethasone, betamethasone, beclomethasone, budesonide, triamcinolone, cloprednol, deflazacort, fluocortolone, cortivazol, paramethasone, fluticasone, fluticasone propionate, triamcinolone acetonide, as well as combinations and/or pharmaceutically acceptable derivatives thereof. In the context of the present invention, the different GCs may be used alone or in combination. Dexamethasone, prednisone and prednisolone are preferred embodiments of GCs.

It is envisaged that all substances which already are or will be classified as a “glucocorticoid” may be employed in the context of the present invention as well. Such future glucocorticoids include compounds which specifically bind to and activate the glucocorticoid receptor. The term “specifically binds to the GC receptor” means in accordance with the present invention that the GC (or a compound which is assumed to act like a GC) associates with (e.g., interacts with) the GC receptor (also known as NR3C1) to a statistically significant degree as compared to association with proteins/receptors generally (i.e., non-specific binding). When the GC receptor binds to glucocorticoids, its primary mechanism of action is the regulation of gene transcription. In the absence of GC, the glucocorticoid receptor (GR) resides in the cytosol complexed with a variety of proteins including heat shock protein 90 (hsp90), heat shock protein 70 (hsp70) and the protein FKBP52 (FK506-binding protein 52). Binding of the GC to the glucocorticoid receptor results in release of the heat shock proteins. It is thus envisaged that a future GC, or a pharmaceutically acceptable derivative or salt of a GC, is able to bind to the GC receptor and to release the above mentioned heat shock proteins. The activated GR complex then up-regulates the expression of anti-inflammatory proteins in the nucleus or represses the expression of pro-inflammatory proteins in the cytosol by preventing the translocation of other transcription factors from the cytosol into the nucleus.

The antibody construct of the present invention comprises a first domain which binds to BCMA and a second domain which binds to CD3.

The term “antibody construct” refers to a molecule in which the structure and/or function is/are based on the structure and/or function of an antibody, e.g., of a full-length immunoglobulin molecule. An antibody construct hence immunospecifically binds to its target or antigen, and/or it comprises domains which are derived from or which are the heavy chain variable region (VH) and/or the light chain variable region (VL) of an antibody. Furthermore, an antibody construct according to the invention comprises the minimum structural requirements of an antibody which allow for immunospecific target binding. This minimum requirement may e.g. be defined by the presence of at least three light chain CDRs (i.e. CDR1, CDR2 and CDR3 of the VL region) and/or three heavy chain CDRs (i.e. CDR1, CDR2 and CDR3 of the VH region), preferably of all six CDRs.

Within the definition of “antibody” according to the invention are full-length antibodies, also including camelid antibodies and other immunoglobulins generated by biotechnological or protein engineering methods or processes. These full-length antibodies may be for example monoclonal, recombinant, chimeric, deimmunized, humanized and human antibodies, as well as antibodies from other species such as mouse, hamster, rabbit, rat, goat, or non-human primates.

“Antibody constructs” of the present invention may have the general structure of a full-length immunoglobulin as it occurs naturally. For example, they may comprise (at least) two full-length antibody heavy chains and two full-length antibody light chains. However, given that the antibody constructs according to the invention comprise one domain binding to BCMA and another domain binding to CD3, they do not occur naturally, and they are markedly different in their function from naturally occurring products. An antibody construct of the invention is hence an artificial “hybrid” molecule comprising at least two distinct binding domains with different specificities.

“Antibody constructs” of the present invention may also comprise fragments of full-length antibodies, such as VH, VHH, VL, (s)dAb, Fv, light chain (VL-CL), Fd (VH-CH1), heavy chain, Fab, Fab′, F(ab′)₂ or “r IgG” (“half antibody” consisting of a heavy chain and a light chain). Antibody constructs according to the invention may also comprise modified fragments of antibodies, also called antibody variants or antibody derivatives. Examples include, but are not limited to, scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “minibodies” exemplified by a structure which is as follows: (VH-VL-CH3)₂, (scFv-CH3)₂, ((scFv)₂-CH3 +CH3), ((scFv)₂-CH3) or (scFv-CH3-scFv)₂, multibodies such as triabodies or tetrabodies, and single domain antibodies such as nanobodies or single variable domain antibodies comprising merely one variable region, which might be VHH, VH or VL, that specifically binds to an antigen or target independently of other variable regions or domains. Further possible formats of the antibody constructs according to the invention are cross bodies, maxi bodies, hetero Fc constructs, mono Fc constructs and scFc constructs.

Furthermore, the definition of the term “antibody construct” includes bivalent and polyvalent/multivalent constructs, which specifically bind to two, three or more antigenic structures, through distinct binding domains. An antibody construct can hence have more binding valences than specificities, e.g. in a case where it has two binding domains for the first target (BCMA) and one binding domain for the second target (CD3)—or vice versa—in which case the construct is trivalent and bispecific. Moreover, the definition of the term “antibody construct” includes molecules consisting of only one polypeptide chain as well as molecules consisting of two, three, four or more polypeptide chains, which chains can be either identical (homodimers, homotrimers or homo oligomers) or different (heterodimer, heterotrimer or heterooligomer). Examples for the above identified antibodies and their fragments, variants, derivatives and antibody constructs derived therefrom are described inter alia in Harlow and Lane, Antibodies: A laboratory manual, CSHL Press (1988); Kontermann and Dübel, Antibody Engineering, Springer, 2nd ed. 2010; and Little, Recombinant Antibodies for Immunotherapy, Cambridge University Press 2009. It is envisaged that the antibody construct is a single chain antibody construct/a single chain polypeptide.

The term “binding domain” or “domain which binds to . . . ” characterizes in connection with the present invention a domain of the antibody construct which immunospecifically binds to/interacts with/recognizes a given epitope on the target or antigen (here: BCMA in the case of the first domain, and CD3 in the case of the second domain). The structure and function of the first domain (binding to BCMA), and preferably also the structure and/or function of the second domain (binding to CD3), is/are based on the structure and/or function of an antibody, e.g. of a full-length immunoglobulin molecule. The “binding domain” or “domain which binds to . . . ” may hence comprise the minimum structural requirements of an antibody which allow for immunospecific target binding. This minimum structural requirement of the first domain may e.g. be defined by the presence of at least three light chain CDRs (i.e. CDR1, CDR2 and CDR3 of the VL region) and/or of three heavy chain CDRs (i.e. CDR1, CDR2 and CDR3 of the VH region), preferably of all six CDRs. It is envisaged that the second domain also comprises this minimum structural requirement of an antibody which allow for the immunospecific target binding. More preferably, the second domain also comprises at least three light chain CDRs (i.e. CDR1, CDR2 and CDR3 of the VL region) and/or three heavy chain CDRs (i.e. CDR1, CDR2 and CDR3 of the VH region), preferably all six CDRs. A “domain which binds to” (or a “binding domain”) may typically comprise an antibody light chain variable region (VL) and an antibody heavy chain variable region (VH); however, it does not have to comprise both, but may comprise only one of VH or VL. Fd fragments, for example, often retain some antigen-binding function of the intact antigen-binding domain.

Examples for the format of a “domain which binds to” (or a “binding domain”) include, but are not limited to, full-length antibodies, fragments of full-length antibodies (such as VH, VHH, VL), (s)dAb, Fv, light chain (VL-CL), Fd (VH-CH1), heavy chain, Fab, Fab′, F(ab′)₂ or “r IgG” (“half antibody”)), antibody variants or derivatives such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “minibodies” (selected from formats such as (VH-VL-CH3)₂, (scFv-CH3)₂, ((scFv)₂-CH3+CH3)), ((scFv)₂-CH3) or (scFv-CH3-scFv)₂, multibodies such as triabodies or tetrabodies, and single domain antibodies such as nanobodies or single variable domain antibodies comprising merely one variable region, which might be VHH, VH or VL. Further examples for the format of a “domain which binds to” (or a “binding domain”) include (1) an antibody fragment or variant comprising VL, VH, CL and CH1 (such as Fab); (2) an antibody fragment or variant comprising two linked Fab fragments (such as a F(ab′)₂); (3) an antibody fragment or variant comprising VH and CH₁ (such as Fd); (4) an antibody fragment or variant comprising VL and CL (such as the light chain); (5) an antibody fragment or variant comprising VL and VH (such as Fv); (5) a dAb fragment (Ward et al., (1989) Nature 341:544-546), which has a VH domain; (6) an antibody variant comprising at least three isolated CDRs of the heavy and/or the light chain; and (7) a single chain Fv (scFv). Examples for embodiments of antibody constructs or binding domains according to the invention are e.g. described in WO 00/006605, WO 2005/040220, WO 2008/119567, WO 2010/037838, WO 2013/026837, WO 2013/026833, US 2014/0308285, US 2014/0302037, W O2014/144722, WO 2014/151910, and WO 2015/048272.

It is envisaged that the antibody construct according to the invention may have a molecular weight of about 20 to about 90 kDa, about 30 to about 80 kDa, about 40 to about 70 kDa, about 50 to about 60 kDa, about 52 to about 58 kDa, and preferably about 54 to about 56 kDa.

The terms “(specifically or immunospecifically) binds to”, “(specifically or immunospecifically) recognizes”, or “(specifically or immunospecifically) reacts with” mean in accordance with this invention that an antibody construct or a binding domain interacts or (immuno-)specifically interacts with a given epitope on the target molecule (antigen), here: BCMA and CD3, respectively. This interaction or association occurs more frequently, more rapidly, with greater duration, with greater affinity, or with some combination of the aforementioned, to an epitope on the specific target than to alternative substances (non-target molecules). Because of the sequence similarity between homologous proteins in different species, an antibody construct or a binding domain that immunspecifically binds to its target (such as a human target) may, however, cross-react with homologous target molecules from different species (such as, from non-human primates, e.g. macaque). The term “specific/immunospecific binding” can hence include the binding of an antibody construct or binding domain to epitopes or structurally related epitopes in more than one species.

In the context of the present invention, the term “epitope” refers to the part or region of the antigen that is recognized/immunospecifically recognized by the binding domain. An “epitope” is antigenic, and thus the term epitope is sometimes also referred to as “antigenic structure” or “antigenic determinant”. The part of the binding domain that binds to the epitope is called a paratope. Specific binding is believed to be accomplished by specific motifs in the amino acid sequence of the binding domain and the antigen. Thus, binding is achieved as a result of their primary, secondary and/or tertiary structure as well as the result of potential secondary modifications of said structures. The specific interaction of the paratope with its antigenic determinant may result in a simple binding of said site to the antigen. In some cases, the specific interaction may alternatively or additionally result in the initiation of a signal, e.g. due to the induction of a change of the conformation of the antigen, an oligomerization of the antigen, etc.

The epitopes of protein antigens are divided into two categories, conformational epitopes and linear epitopes, based on their structure and interaction with the paratope. A conformational epitope is composed of discontinuous sections of the antigen's amino acid sequence. These epitopes interact with the paratope based on the three-dimensional surface features and shape or tertiary structure (folding) of the antigen. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy. By contrast, linear epitopes interact with the paratope based on their primary structure. A linear epitope is formed by a continuous sequence of amino acids from the antigen and typically includes at least 3 or at least 4, and more usually, at least 5 or at least 6 or at least 7, for example, about 8 to about 10 amino acids in a unique sequence.

The interaction between the binding domain and the epitope of the target antigen implies that a binding domain exhibits appreciable or significant affinity for the epitope/the target antigen (here: BCMA and CD3, respectively). In general, it does furthermore not exhibit significant affinity for proteins or antigens other than the target antigen (here: BCMA/CD3)—notwithstanding the above discussed cross-reactivity with homologous targets e.g. from other species. “Significant affinity” includes binding with an affinity (dissociation constant, KD) of ≤10⁻⁶ M. Preferably, binding is considered specific when the binding affinity is ≤10⁻⁷ M, ≤10⁻⁸ M, ≤10⁻⁹ M, ≤10⁻¹⁰ M, or even ≤10 ⁻¹¹ M, or ≤10⁻¹² M. Whether a binding domain (immuno-)specifically reacts with or binds to a target can be tested readily e.g. by comparing the affinity of said binding domain to its desired target protein or antigen with the affinity of said binding domain to non-target proteins or antigens (here: proteins other than BCMA or CD3, respectively). Preferably, an antibody construct of the invention does not significantly bind to proteins or antigens other than BCMA or CD3, respectively (i.e., the first domain does not bind to proteins other than BCMA and the second domain does not bind to proteins other than CD3). For example, it is envisaged that the antibody construct of the invention (and more specifically its first domain) does not significantly bind to, interact with, recognize or cross-react with human BAFF-R and/or human TACI.

The equilibrium dissociation constant (KD) of an antibody construct of the invention to BCMA can be determined by Scatchard or by biacore analysis, as described e.g. in WO 2013/072406. The KD values for CD3 can e.g. be determined by surface plasmon resonance analysis, as described e.g. in WO 2013/072406. It is envisaged that the antibody construct of the present invention has a KD value for BCMA and/or for CD3 in the 2-digit or 1-digit nanomolar range or in the three digit or even two digit picomolar range.

The term “does not significantly bind” means that an antibody construct or binding domain of the present invention does not bind a protein or antigen other than BCMA or CD3, i.e., shows reactivity of ≤30%, preferably ≤20%, more preferably ≤10%, particularly preferably ≤9%, 8%, 7%, 6% or 5% with proteins or antigens other than BCMA or CD3, whereby binding to BCMA or CD3, respectively, is set to be 100%.

According to one embodiment of the antibody construct of the present invention, the first and/or the second domain are in the format of an scFv. In an scFv, the VH region and the and VL region are arranged in the order VH-VL or VL-VH (from N- to C-terminus). It is envisaged that the VH and the VL regions of the first and/or the second binding domain are connected via a linker, preferably a peptide linker. According to one embodiment of the first and/or the second domain, the VH-region is positioned N-terminally of the linker, and the VL-region is positioned C-terminally of the linker. It is furthermore envisaged that the first domain and the second domain of the antibody construct are connected via a linker, preferably a peptide linker. The linkers are preferably peptide linkers, more preferably short peptide linkers. Examples are shown in SEQ ID NOs: 686-699. In the present context, a “short” linker has between 2 and 50 amino acids, preferably between 3 and 35, between 4 and 30, between 5 and 25, between 6 and 20 or between 6 and 17 amino acids. The linker between two variable regions of one binding domain may have a different length (e.g. may be longer) than the linker between the two binding domains. For example, the linker between two variable regions of one binding domain may have a length between 7 and 15 amino acids, preferably between 9 and 13, and the linker between the two binding domains may have a length between 3 and 10 amino acids, preferably between 4 and 8. It is further envisaged that the peptide linkers are glycine/serine linkers, such as those depicted in SEQ ID NOs: 687, 689-699.

It is envisaged for the antibody construct of the present invention that
a) the antibody construct is a single chain polypeptide,
b) the first domain is in the format of an scFv,
c) the second domain is in the format of an scFv, and/or
d) the first and the second domain are connected via a linker, preferably a peptide linker, more preferably a glycine/serine linker.

The first domain of the antibody construct of the invention binds to BCMA (B cell maturation antigen, TNFRSF17, CD269). More preferably, it binds to BCMA on the surface of a target cell. The “target cell” can be any prokaryotic or eukaryotic cell expressing BCMA on its surface; preferably the target cell is a cell that is part of the human or animal body, such as a specific BCMA expressing cancer or tumor cell or a cell of a BCMA positive neoplasm. It is furthermore envisaged that the first domain binds to human BCMA, preferably to human BCMA on the surface of a target cell. It is also envisaged that the first domain binds to macaque BCMA, preferably to macaque BCMA on the surface of a target cell. A preferred amino acid sequence for human BCMA is depicted in SEQ ID NO: 647, for macaque BCMA in SEQ ID NO: 648, for the extracellular domain of human BCMA in SEQ ID NO: 649, for the extracellular domain of macaque BCMA in SEQ ID NO: 650.

In one embodiment of the present invention, the first domain of the antibody construct binds to epitope cluster 3 of BCMA. More preferably, it binds to epitope cluster 3 of human BCMA. A preferred amino acid sequence for epitope cluster 3 of human BCMA is depicted in SEQ ID NO: 651. Antibody constructs having domains that bind to said epitope cluster 3 of BCMA are described in detail in WO 2013/072406, the content of which is hereby incorporated by reference. These antibody constructs are shown in WO 2013/072406 to have a very beneficial epitope/activity relationship.

A method for BCMA epitope mapping is used in WO 2013/072406 and described in the following: One or more pre-defined regions (each in the form of a contiguous amino acid stretch) within the extracellular domain of human BCMA is exchanged/replaced with the corresponding region of a rodent BCMA molecule (such as murine BCMA, but other rodent species are also conceivable, so long as the binding domain is not cross-reactive with the rodent species used). These human BCMA/rodent (murine) BCMA chimeras are expressed on the surface of host cells (such as CHO cells). Binding of the antibody or antibody construct can be tested via FACS analysis. When the binding of the antibody or antibody construct to the chimeric molecule is entirely abolished, or when a significant binding decrease is observed, it can be concluded that the region of human BCMA which was removed from this chimeric molecule is relevant for the immunospecific epitope-paratope recognition. Said decrease in binding is preferably at least 10%, 20%, 30%, 40%, or 50%; more preferably at least 60%, 70%, or 80%, and most preferably 90%, 95% or even 100% in comparison to the binding to human (wild-type) BCMA, whereby binding to human BCMA is set to be 100%. Alternatively or additionally, the above described epitope mapping analysis can be modified by introducing one or more point mutations into the sequence of the extracellular domain of BCMA.

It is furthermore envisaged for the antibody construct of the present invention that the first domain which binds to BCMA comprises a VH region comprising CDR-H1, CDR-H2 and CDR-H3 and a VL region comprising CDR-L1, CDR-L2 and CDR-L3 selected from:
(1) CDR-H1 as depicted in SEQ ID NO: 1, CDR-H2 as depicted in SEQ ID NO: 2, CDR-H3 as depicted in SEQ ID NO: 3, CDR-L1 as depicted in SEQ ID NO: 4, CDR-L2 as depicted in SEQ ID NO: 5, and CDR-L3 as depicted in SEQ ID NO: 6;
(2) CDR-H1 as depicted in SEQ ID NO: 11, CDR-H2 as depicted in SEQ ID NO: 12, CDR-H3 as depicted in SEQ ID NO: 13, CDR-L1 as depicted in SEQ ID NO: 14, CDR-L2 as depicted in SEQ ID NO: 15, and CDR-L3 as depicted in SEQ ID NO: 16;
(3) CDR-H1 as depicted in SEQ ID NO: 21, CDR-H2 as depicted in SEQ ID NO: 22, CDR-H3 as depicted in SEQ ID NO: 23, CDR-L1 as depicted in SEQ ID NO: 24, CDR-L2 as depicted in SEQ ID NO: 25, and CDR-L3 as depicted in SEQ ID NO: 26;
(4) CDR-H1 as depicted in SEQ ID NO: 31, CDR-H2 as depicted in SEQ ID NO: 32, CDR-H3 as depicted in SEQ ID NO: 33, CDR-L1 as depicted in SEQ ID NO: 34, CDR-L2 as depicted in SEQ ID NO: 35, and CDR-L3 as depicted in SEQ ID NO: 36;
(5) CDR-H1 as depicted in SEQ ID NO: 41, CDR-H2 as depicted in SEQ ID NO: 42, CDR-H3 as depicted in SEQ ID NO: 43, CDR-L1 as depicted in SEQ ID NO: 44, CDR-L2 as depicted in SEQ ID NO: 45, and CDR-L3 as depicted in SEQ ID NO: 46;
(6) CDR-H1 as depicted in SEQ ID NO: 51, CDR-H2 as depicted in SEQ ID NO: 52, CDR-H3 as depicted in SEQ ID NO: 53, CDR-L1 as depicted in SEQ ID NO: 54, CDR-L2 as depicted in SEQ ID NO: 55, and CDR-L3 as depicted in SEQ ID NO: 56;
(7) CDR-H1 as depicted in SEQ ID NO: 61, CDR-H2 as depicted in SEQ ID NO: 62, CDR-H3 as depicted in SEQ ID NO: 63, CDR-L1 as depicted in SEQ ID NO: 64, CDR-L2 as depicted in SEQ ID NO: 65, and CDR-L3 as depicted in SEQ ID NO: 66;
(8) CDR-H1 as depicted in SEQ ID NO: 71, CDR-H2 as depicted in SEQ ID NO: 72, CDR-H3 as depicted in SEQ ID NO: 73, CDR-L1 as depicted in SEQ ID NO: 74, CDR-L2 as depicted in SEQ ID NO: 75, and CDR-L3 as depicted in SEQ ID NO: 76;
(9) CDR-H1 as depicted in SEQ ID NO: 81, CDR-H2 as depicted in SEQ ID NO: 82, CDR-H3 as depicted in SEQ ID NO: 83, CDR-L1 as depicted in SEQ ID NO: 84, CDR-L2 as depicted in SEQ ID NO: 85, and CDR-L3 as depicted in SEQ ID NO: 86;
(10) CDR-H1 as depicted in SEQ ID NO: 91, CDR-H2 as depicted in SEQ ID NO: 92, CDR-H3 as depicted in SEQ ID NO: 93, CDR-L1 as depicted in SEQ ID NO: 94, CDR-L2 as depicted in SEQ ID NO: 95, and CDR-L3 as depicted in SEQ ID NO: 96;
(11) CDR-H1 as depicted in SEQ ID NO: 101, CDR-H2 as depicted in SEQ ID NO: 102, CDR-H3 as depicted in SEQ ID NO: 103, CDR-L1 as depicted in SEQ ID NO: 104, CDR-L2 as depicted in SEQ ID NO: 105, and CDR-L3 as depicted in SEQ ID NO: 106;
(12) CDR-H1 as depicted in SEQ ID NO: 111, CDR-H2 as depicted in SEQ ID NO: 112, CDR-H3 as depicted in SEQ ID NO: 113, CDR-L1 as depicted in SEQ ID NO: 114, CDR-L2 as depicted in SEQ ID NO: 115, and CDR-L3 as depicted in SEQ ID NO: 116;
(13) CDR-H1 as depicted in SEQ ID NO: 121, CDR-H2 as depicted in SEQ ID NO: 122, CDR-H3 as depicted in SEQ ID NO: 123, CDR-L1 as depicted in SEQ ID NO: 124, CDR-L2 as depicted in SEQ ID NO: 125, and CDR-L3 as depicted in SEQ ID NO: 126;
(14) CDR-H1 as depicted in SEQ ID NO: 131, CDR-H2 as depicted in SEQ ID NO: 132, CDR-H3 as depicted in SEQ ID NO: 133, CDR-L1 as depicted in SEQ ID NO: 134, CDR-L2 as depicted in SEQ ID NO: 135, and CDR-L3 as depicted in SEQ ID NO: 136;
(15) CDR-H1 as depicted in SEQ ID NO: 141, CDR-H2 as depicted in SEQ ID NO: 142, CDR-H3 as depicted in SEQ ID NO: 143, CDR-L1 as depicted in SEQ ID NO: 144, CDR-L2 as depicted in SEQ ID NO: 145, and CDR-L3 as depicted in SEQ ID NO: 146;
(16) CDR-H1 as depicted in SEQ ID NO: 151, CDR-H2 as depicted in SEQ ID NO: 152, CDR-H3 as depicted in SEQ ID NO: 153, CDR-L1 as depicted in SEQ ID NO: 154, CDR-L2 as depicted in SEQ ID NO: 155, and CDR-L3 as depicted in SEQ ID NO: 156;
(17) CDR-H1 as depicted in SEQ ID NO: 161, CDR-H2 as depicted in SEQ ID NO: 162, CDR-H3 as depicted in SEQ ID NO: 163, CDR-L1 as depicted in SEQ ID NO: 164, CDR-L2 as depicted in SEQ ID NO: 165, and CDR-L3 as depicted in SEQ ID NO: 166;
(18) CDR-H1 as depicted in SEQ ID NO: 171, CDR-H2 as depicted in SEQ ID NO: 172, CDR-H3 as depicted in SEQ ID NO: 173, CDR-L1 as depicted in SEQ ID NO: 174, CDR-L2 as depicted in SEQ ID NO: 175, and CDR-L3 as depicted in SEQ ID NO: 176;
(19) CDR-H1 as depicted in SEQ ID NO: 181, CDR-H2 as depicted in SEQ ID NO: 182, CDR-H3 as depicted in SEQ ID NO: 183, CDR-L1 as depicted in SEQ ID NO: 184, CDR-L2 as depicted in SEQ ID NO: 185, and CDR-L3 as depicted in SEQ ID NO: 186;
(20) CDR-H1 as depicted in SEQ ID NO: 191, CDR-H2 as depicted in SEQ ID NO: 192, CDR-H3 as depicted in SEQ ID NO: 193, CDR-L1 as depicted in SEQ ID NO: 194, CDR-L2 as depicted in SEQ ID NO: 195, and CDR-L3 as depicted in SEQ ID NO: 196;
(21) CDR-H1 as depicted in SEQ ID NO: 201, CDR-H2 as depicted in SEQ ID NO: 202, CDR-H3 as depicted in SEQ ID NO: 203, CDR-L1 as depicted in SEQ ID NO: 204, CDR-L2 as depicted in SEQ ID NO: 205, and CDR-L3 as depicted in SEQ ID NO: 206;
(22) CDR-H1 as depicted in SEQ ID NO: 211, CDR-H2 as depicted in SEQ ID NO: 212, CDR-H3 as depicted in SEQ ID NO: 213, CDR-L1 as depicted in SEQ ID NO: 214, CDR-L2 as depicted in SEQ ID NO: 215, and CDR-L3 as depicted in SEQ ID NO: 216;
(23) CDR-H1 as depicted in SEQ ID NO: 221, CDR-H2 as depicted in SEQ ID NO: 222, CDR-H3 as depicted in SEQ ID NO: 223, CDR-L1 as depicted in SEQ ID NO: 224, CDR-L2 as depicted in SEQ ID NO: 225, and CDR-L3 as depicted in SEQ ID NO: 226;
(24) CDR-H1 as depicted in SEQ ID NO: 231, CDR-H2 as depicted in SEQ ID NO: 232, CDR-H3 as depicted in SEQ ID NO: 233, CDR-L1 as depicted in SEQ ID NO: 234, CDR-L2 as depicted in SEQ ID NO: 235, and CDR-L3 as depicted in SEQ ID NO: 236;
(25) CDR-H1 as depicted in SEQ ID NO: 241, CDR-H2 as depicted in SEQ ID NO: 242, CDR-H3 as depicted in SEQ ID NO: 243, CDR-L1 as depicted in SEQ ID NO: 244, CDR-L2 as depicted in SEQ ID NO: 245, and CDR-L3 as depicted in SEQ ID NO: 246;
(26) CDR-H1 as depicted in SEQ ID NO: 251, CDR-H2 as depicted in SEQ ID NO: 252, CDR-H3 as depicted in SEQ ID NO: 253, CDR-L1 as depicted in SEQ ID NO: 254, CDR-L2 as depicted in SEQ ID NO: 255, and CDR-L3 as depicted in SEQ ID NO: 256;
(27) CDR-H1 as depicted in SEQ ID NO: 261, CDR-H2 as depicted in SEQ ID NO: 262, CDR-H3 as depicted in SEQ ID NO: 263, CDR-L1 as depicted in SEQ ID NO: 264, CDR-L2 as depicted in SEQ ID NO: 265, and CDR-L3 as depicted in SEQ ID NO: 266;
(28) CDR-H1 as depicted in SEQ ID NO: 271, CDR-H2 as depicted in SEQ ID NO: 272, CDR-H3 as depicted in SEQ ID NO: 273, CDR-L1 as depicted in SEQ ID NO: 274, CDR-L2 as depicted in SEQ ID NO: 275, and CDR-L3 as depicted in SEQ ID NO: 276;
(29) CDR-H1 as depicted in SEQ ID NO: 281, CDR-H2 as depicted in SEQ ID NO: 282, CDR-H3 as depicted in SEQ ID NO: 283, CDR-L1 as depicted in SEQ ID NO: 284, CDR-L2 as depicted in SEQ ID NO: 285, and CDR-L3 as depicted in SEQ ID NO: 286;
(30) CDR-H1 as depicted in SEQ ID NO: 291, CDR-H2 as depicted in SEQ ID NO: 292, CDR-H3 as depicted in SEQ ID NO: 293, CDR-L1 as depicted in SEQ ID NO: 294, CDR-L2 as depicted in SEQ ID NO: 295, and CDR-L3 as depicted in SEQ ID NO: 296;
(31) CDR-H1 as depicted in SEQ ID NO: 301, CDR-H2 as depicted in SEQ ID NO: 302, CDR-H3 as depicted in SEQ ID NO: 303, CDR-L1 as depicted in SEQ ID NO: 304, CDR-L2 as depicted in SEQ ID NO: 305, and CDR-L3 as depicted in SEQ ID NO: 306;
(32) CDR-H1 as depicted in SEQ ID NO: 311, CDR-H2 as depicted in SEQ ID NO: 312, CDR-H3 as depicted in SEQ ID NO: 313, CDR-L1 as depicted in SEQ ID NO: 314, CDR-L2 as depicted in SEQ ID NO: 315, and CDR-L3 as depicted in SEQ ID NO: 316;
(33) CDR-H1 as depicted in SEQ ID NO: 321, CDR-H2 as depicted in SEQ ID NO: 322, CDR-H3 as depicted in SEQ ID NO: 323, CDR-L1 as depicted in SEQ ID NO: 324, CDR-L2 as depicted in SEQ ID NO: 325, and CDR-L3 as depicted in SEQ ID NO: 326;
(34) CDR-H1 as depicted in SEQ ID NO: 331, CDR-H2 as depicted in SEQ ID NO: 332, CDR-H3 as depicted in SEQ ID NO: 333, CDR-L1 as depicted in SEQ ID NO: 334, CDR-L2 as depicted in SEQ ID NO: 335, and CDR-L3 as depicted in SEQ ID NO: 336;
(35) CDR-H1 as depicted in SEQ ID NO: 341, CDR-H2 as depicted in SEQ ID NO: 342, CDR-H3 as depicted in SEQ ID NO: 343, CDR-L1 as depicted in SEQ ID NO: 344, CDR-L2 as depicted in SEQ ID NO: 345, and CDR-L3 as depicted in SEQ ID NO: 346;
(36) CDR-H1 as depicted in SEQ ID NO: 351, CDR-H2 as depicted in SEQ ID NO: 352, CDR-H3 as depicted in SEQ ID NO: 353, CDR-L1 as depicted in SEQ ID NO: 354, CDR-L2 as depicted in SEQ ID NO: 355, and CDR-L3 as depicted in SEQ ID NO: 356;
(37) CDR-H1 as depicted in SEQ ID NO: 361, CDR-H2 as depicted in SEQ ID NO: 362, CDR-H3 as depicted in SEQ ID NO: 363, CDR-L1 as depicted in SEQ ID NO: 364, CDR-L2 as depicted in SEQ ID NO: 365, and CDR-L3 as depicted in SEQ ID NO: 366;
(38) CDR-H1 as depicted in SEQ ID NO: 371, CDR-H2 as depicted in SEQ ID NO: 372, CDR-H3 as depicted in SEQ ID NO: 373, CDR-L1 as depicted in SEQ ID NO: 374, CDR-L2 as depicted in SEQ ID NO: 375, and CDR-L3 as depicted in SEQ ID NO: 376;
(39) CDR-H1 as depicted in SEQ ID NO: 381, CDR-H2 as depicted in SEQ ID NO: 382, CDR-H3 as depicted in SEQ ID NO: 383, CDR-L1 as depicted in SEQ ID NO: 384, CDR-L2 as depicted in SEQ ID NO: 385, and CDR-L3 as depicted in SEQ ID NO: 386;
(40) CDR-H1 as depicted in SEQ ID NO: 391, CDR-H2 as depicted in SEQ ID NO: 392, CDR-H3 as depicted in SEQ ID NO: 393, CDR-L1 as depicted in SEQ ID NO: 394, CDR-L2 as depicted in SEQ ID NO: 395, and CDR-L3 as depicted in SEQ ID NO: 396;
(41) CDR-H1 as depicted in SEQ ID NO: 401, CDR-H2 as depicted in SEQ ID NO: 402, CDR-H3 as depicted in SEQ ID NO: 403, CDR-L1 as depicted in SEQ ID NO: 404, CDR-L2 as depicted in SEQ ID NO: 405, and CDR-L3 as depicted in SEQ ID NO: 406;
(42) CDR-H1 as depicted in SEQ ID NO: 411, CDR-H2 as depicted in SEQ ID NO: 412, CDR-H3 as depicted in SEQ ID NO: 413, CDR-L1 as depicted in SEQ ID NO: 414, CDR-L2 as depicted in SEQ ID NO: 415, and CDR-L3 as depicted in SEQ ID NO: 416;
(43) CDR-H1 as depicted in SEQ ID NO: 421, CDR-H2 as depicted in SEQ ID NO: 422, CDR-H3 as depicted in SEQ ID NO: 423, CDR-L1 as depicted in SEQ ID NO: 424, CDR-L2 as depicted in SEQ ID NO: 425, and CDR-L3 as depicted in SEQ ID NO: 426;
(44) CDR-H1 as depicted in SEQ ID NO: 431, CDR-H2 as depicted in SEQ ID NO: 432, CDR-H3 as depicted in SEQ ID NO: 433, CDR-L1 as depicted in SEQ ID NO: 434, CDR-L2 as depicted in SEQ ID NO: 435, and CDR-L3 as depicted in SEQ ID NO: 436;
(45) CDR-H1 as depicted in SEQ ID NO: 441, CDR-H2 as depicted in SEQ ID NO: 442, CDR-H3 as depicted in SEQ ID NO: 443, CDR-L1 as depicted in SEQ ID NO: 444, CDR-L2 as depicted in SEQ ID NO: 445, and CDR-L3 as depicted in SEQ ID NO: 446;
(46) CDR-H1 as depicted in SEQ ID NO: 451, CDR-H2 as depicted in SEQ ID NO: 452, CDR-H3 as depicted in SEQ ID NO: 453, CDR-L1 as depicted in SEQ ID NO: 454, CDR-L2 as depicted in SEQ ID NO: 455, and CDR-L3 as depicted in SEQ ID NO: 456;
(47) CDR-H1 as depicted in SEQ ID NO: 461, CDR-H2 as depicted in SEQ ID NO: 462, CDR-H3 as depicted in SEQ ID NO: 463, CDR-L1 as depicted in SEQ ID NO: 464, CDR-L2 as depicted in SEQ ID NO: 465, and CDR-L3 as depicted in SEQ ID NO: 466;
(48) CDR-H1 as depicted in SEQ ID NO: 471, CDR-H2 as depicted in SEQ ID NO: 472, CDR-H3 as depicted in SEQ ID NO: 473, CDR-L1 as depicted in SEQ ID NO: 474, CDR-L2 as depicted in SEQ ID NO: 475, and CDR-L3 as depicted in SEQ ID NO: 476;
(49) CDR-H1 as depicted in SEQ ID NO: 481, CDR-H2 as depicted in SEQ ID NO: 482, CDR-H3 as depicted in SEQ ID NO: 483, CDR-L1 as depicted in SEQ ID NO: 484, CDR-L2 as depicted in SEQ ID NO: 485, and CDR-L3 as depicted in SEQ ID NO: 486;
(50) CDR-H1 as depicted in SEQ ID NO: 491, CDR-H2 as depicted in SEQ ID NO: 492, CDR-H3 as depicted in SEQ ID NO: 493, CDR-L1 as depicted in SEQ ID NO: 494, CDR-L2 as depicted in SEQ ID NO: 495, and CDR-L3 as depicted in SEQ ID NO: 496;
(51) CDR-H1 as depicted in SEQ ID NO: 501, CDR-H2 as depicted in SEQ ID NO: 502, CDR-H3 as depicted in SEQ ID NO: 503, CDR-L1 as depicted in SEQ ID NO: 504, CDR-L2 as depicted in SEQ ID NO: 505, and CDR-L3 as depicted in SEQ ID NO: 506;
(52) CDR-H1 as depicted in SEQ ID NO: 511, CDR-H2 as depicted in SEQ ID NO: 512, CDR-H3 as depicted in SEQ ID NO: 513, CDR-L1 as depicted in SEQ ID NO: 514, CDR-L2 as depicted in SEQ ID NO: 515, and CDR-L3 as depicted in SEQ ID NO: 516; and
(53) CDR-H1 as depicted in SEQ ID NO: 521, CDR-H2 as depicted in SEQ ID NO: 522, CDR-H3 as depicted in SEQ ID NO: 523, CDR-L1 as depicted in SEQ ID NO: 524, CDR-L2 as depicted in SEQ ID NO: 525, and CDR-L3 as depicted in SEQ ID NO: 526.

It is also envisaged for the antibody construct of the present invention that the first domain which binds to BCMA comprises a VL region having an amino acid sequence selected from the group consisting of those depicted in SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, and 528. It is envisaged that the first domain comprises a VL region having an amino acid sequence as depicted in SEQ ID NO: 178.

It is furthermore envisaged that the first domain which binds to BCMA comprises a VH region having an amino acid sequence selected from the group consisting of those depicted in SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 307, 317, 327, 337, 347, 357, 367, 377, 387, 397, 407, 417, 427, 437, 447, 457, 467, 477, 487, 497, 507, 517, and 527. It is envisaged that the first domain comprises a VL region having an amino acid sequence as depicted in SEQ ID NO: 177.

It is also envisaged for the antibody construct of the present invention that the first domain which binds to BCMA comprises a VH region and a VL region selected from the group consisting of:
(1) a VH region as depicted in SEQ ID NO: 7 and a VL region as depicted in SEQ ID NO: 8;
(2) a VH region as depicted in SEQ ID NO: 17 and a VL region as depicted in SEQ ID NO: 18;
(3) a VH region as depicted in SEQ ID NO: 27 and a VL region as depicted in SEQ ID NO: 28;
(4) a VH region as depicted in SEQ ID NO: 37 and a VL region as depicted in SEQ ID NO: 38;
(5) a VH region as depicted in SEQ ID NO: 47 and a VL region as depicted in SEQ ID NO: 48;
(6) a VH region as depicted in SEQ ID NO: 57 and a VL region as depicted in SEQ ID NO: 58;
(7) a VH region as depicted in SEQ ID NO: 67 and a VL region as depicted in SEQ ID NO: 68;
(8) a VH region as depicted in SEQ ID NO: 77 and a VL region as depicted in SEQ ID NO: 78;
(9) a VH region as depicted in SEQ ID NO: 87 and a VL region as depicted in SEQ ID NO: 88;
(10) a VH region as depicted in SEQ ID NO: 97 and a VL region as depicted in SEQ ID NO: 98;
(11) a VH region as depicted in SEQ ID NO: 107 and a VL region as depicted in SEQ ID NO: 108;
(12) a VH region as depicted in SEQ ID NO: 117 and a VL region as depicted in SEQ ID NO: 118;
(13) a VH region as depicted in SEQ ID NO: 127 and a VL region as depicted in SEQ ID NO: 128;
(14) a VH region as depicted in SEQ ID NO: 137 and a VL region as depicted in SEQ ID NO: 138;

(15) a VH region as depicted in SEQ ID NO: 147 and a VL region as depicted in SEQ ID NO: 148;
(16) a VH region as depicted in SEQ ID NO: 157 and a VL region as depicted in SEQ ID NO: 158;
(17) a VH region as depicted in SEQ ID NO: 167 and a VL region as depicted in SEQ ID NO: 168;
(18) a VH region as depicted in SEQ ID NO: 177 and a VL region as depicted in SEQ ID NO: 178;
(19) a VH region as depicted in SEQ ID NO: 187 and a VL region as depicted in SEQ ID NO: 188;
(20) a VH region as depicted in SEQ ID NO: 197 and a VL region as depicted in SEQ ID NO: 198;
(21) a VH region as depicted in SEQ ID NO: 207 and a VL region as depicted in SEQ ID NO: 208;
(22) a VH region as depicted in SEQ ID NO: 217 and a VL region as depicted in SEQ ID NO: 218;
(23) a VH region as depicted in SEQ ID NO: 227 and a VL region as depicted in SEQ ID NO: 228;
(24) a VH region as depicted in SEQ ID NO: 237 and a VL region as depicted in SEQ ID NO: 238;
(25) a VH region as depicted in SEQ ID NO: 247 and a VL region as depicted in SEQ ID NO: 248;
(26) a VH region as depicted in SEQ ID NO: 257 and a VL region as depicted in SEQ ID NO: 258;
(27) a VH region as depicted in SEQ ID NO: 267 and a VL region as depicted in SEQ ID NO: 268;
(28) a VH region as depicted in SEQ ID NO: 277 and a VL region as depicted in SEQ ID NO: 278;
(29) a VH region as depicted in SEQ ID NO: 287 and a VL region as depicted in SEQ ID NO: 288;
(30) a VH region as depicted in SEQ ID NO: 297 and a VL region as depicted in SEQ ID NO: 298;
(31) a VH region as depicted in SEQ ID NO: 307 and a VL region as depicted in SEQ ID NO: 308;
(32) a VH region as depicted in SEQ ID NO: 317 and a VL region as depicted in SEQ ID NO: 318;
(33) a VH region as depicted in SEQ ID NO: 327 and a VL region as depicted in SEQ ID NO: 328;
(34) a VH region as depicted in SEQ ID NO: 337 and a VL region as depicted in SEQ ID NO: 338;
(35) a VH region as depicted in SEQ ID NO: 347 and a VL region as depicted in SEQ ID NO: 348;
(36) a VH region as depicted in SEQ ID NO: 357 and a VL region as depicted in SEQ ID NO: 358;
(37) a VH region as depicted in SEQ ID NO: 367 and a VL region as depicted in SEQ ID NO: 368;
(38) a VH region as depicted in SEQ ID NO: 377 and a VL region as depicted in SEQ ID NO: 378;
(39) a VH region as depicted in SEQ ID NO: 387 and a VL region as depicted in SEQ ID NO: 388;
(40) a VH region as depicted in SEQ ID NO: 397 and a VL region as depicted in SEQ ID NO: 398;
(41) a VH region as depicted in SEQ ID NO: 407 and a VL region as depicted in SEQ ID NO: 408;
(42) a VH region as depicted in SEQ ID NO: 417 and a VL region as depicted in SEQ ID NO: 418;
(43) a VH region as depicted in SEQ ID NO: 427 and a VL region as depicted in SEQ ID NO: 428;
(44) a VH region as depicted in SEQ ID NO: 437 and a VL region as depicted in SEQ ID NO: 438;
(45) a VH region as depicted in SEQ ID NO: 447 and a VL region as depicted in SEQ ID NO: 448;
(46) a VH region as depicted in SEQ ID NO: 457 and a VL region as depicted in SEQ ID NO: 458;
(47) a VH region as depicted in SEQ ID NO: 467 and a VL region as depicted in SEQ ID NO: 468;
(48) a VH region as depicted in SEQ ID NO: 477 and a VL region as depicted in SEQ ID NO: 478;
(49) a VH region as depicted in SEQ ID NO: 487 and a VL region as depicted in SEQ ID NO: 488;
(50) a VH region as depicted in SEQ ID NO: 497 and a VL region as depicted in SEQ ID NO: 498;
(51) a VH region as depicted in SEQ ID NO: 507 and a VL region as depicted in SEQ ID NO: 508;
(52) a VH region as depicted in SEQ ID NO: 517 and a VL region as depicted in SEQ ID NO: 518; and
(53) a VH region as depicted in SEQ ID NO: 527 and a VL region as depicted in SEQ ID NO: 528.

It is envisaged that the first domain comprises a VH region having an amino acid sequence as depicted in SEQ ID NO: 177 and a VL region having an amino acid sequence as depicted in SEQ ID NO: 178.

It is also envisaged for the antibody construct of the present invention that the first domain which binds to BCMA comprises or consists of a polypeptide having an amino acid sequence selected from the group consisting of SEQ ID NOs: 9, 19, 29, 39, 49, 59, 69, 79, 89, 109, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 519, and 529. It is envisaged that the first domain comprises or consists of a polypeptide having an amino acid sequence as depicted in SEQ ID NO: 179.

“T cells” or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

“CD3” (cluster of differentiation 3) is a T cell co-receptor composed of four chains. In mammals, the CD3 protein complex contains a CD3γ (gamma) chain, a CD3δ (delta) chain, and two CD3ε (epsilon) chains. These four chains associate with the T cell receptor (TCR) and the so-called ζ (zeta) chain to for the “T cell receptor complex” and to generate an activation signal in T lymphocytes. The CD3γ (gamma), CD3δ (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide which in humans is encoded by the CD3E gene which resides on chromosome 11.

The redirected lysis of target cells via the recruitment of T cells by an antibody construct which binds to CD3 on the T cell and to a target protein on the target cell generally involves cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO 2007/042261.

Cytotoxicity mediated by BCMAxCD3 antibody constructs can be measured in various ways. The “half maximal effective concentration” (EC₅₀) is commonly used as a measure of potency of a biologically active molecule such as an antibody construct of the present invention. It is expressed in molar units. In the present case of measuring cytotoxicity, the EC₅₀ value refers to the concentration of an antibody construct inducing a cytotoxic response (lysis of target cells) halfway between the baseline and the maximum. Effector cells in a cytotoxicity assay can e.g. be stimulated enriched (human) CD8 positive T cells or unstimulated (human) peripheral blood mononuclear cells (PBMC). The target cells should express BCMA on the cell surface. Preferably the target cells express (at least) the extracellular domain of BCMA on the cell surface. Target cells can be a cell line (such as CHO) which is stably or transiently transfected with BCMA, e.g. human BCMA. Alternatively, the target cells can be a BCMA positive natural expresser cell line, such as the human multiple myeloma cell line L363 or NCI-H929.

The effector to target cell (E:T) ratio in a cytotoxicity assay is usually about 10:1, but can also vary. Cytotoxic activity of BCMAxCD3 antibody constructs can be measured in a 51-chromium release assay (e.g. with an incubation time of about 18 hours) or in a in a FACS-based cytotoxicity assay (e.g. with an incubation time of about 48 hours). Modifications of the incubation time (cytotoxic reaction) are also envisaged. Other methods of measuring cytotoxicity are well-known and comprise MTT or MTS assays, ATP-based assays including bioluminescent assays, the sulforhodamine B (SRB) assay, WST assay, clonogenic assay and the ECIS technology.

According to one embodiment, the cytotoxic activity mediated by BCMAxCD3 antibody constructs of the present invention is measured in a FACS-based cytotoxicity assay. It is envisaged that the EC₅₀ value of the BCMAxCD3 antibody constructs is ≤5000 pM or ≤4000 pM, more preferably ≤3000 pM or ≤2000 pM, even more preferably ≤1000 pM or ≤500 pM, even more preferably ≤400 pM or ≤300 pM, even more preferably ≤200 pM, even more preferably ≤100 pM, even more preferably ≤50 pM, even more preferably ≤20 pM or ≤10 pM, and most preferably ≤5 pM. It is also envisaged that the antibody construct of the present invention has an EC50 value in the 3-digit, 2-digit or 1-digit pg/ml range, as determined in a FACS-based cytotoxicity assay. The assay may be carried out with the L363 or NCI-H929 cell line or with BCMA transfected CHO cells as target cells and stimulated enriched (human) CD8 positive T cells or unstimulated (human) PBMC as effector cells. See also WO 2013/072406.

The second domain of the antibody construct of the invention binds to CD3. More preferably, it binds to CD3 on the surface of a T cell. It is furthermore envisaged that the second domain binds to human CD3, preferably to human CD3 on the surface of a T cell. It is also envisaged that the second domain binds to CD3 epsilon. More preferably, it binds to human CD3 epsilon, e.g. to human CD3 epsilon on the surface of a T cell. A preferred amino acid sequence for the extracellular domain of human CD3 epsilon is depicted in SEQ ID NO: 653.

In one embodiment of the present invention, the second domain of the antibody construct binds to human CD3 epsilon (or human CD3 epsilon on the surface of a T cell) and to Callithrix jacchus or Saimiri sciureus CD3 epsilon. It is also envisaged that the second domain binds to an extracellular epitope of CD3 epsilon, preferably to an extracellular epitope of human CD3 epsilon. It is also envisaged that the second domain binds to an extracellular epitope of the human and the Macaca CD3 epsilon chain. One preferred epitope of CD3 epsilon is comprised within amino acid residues 1-27 of the human CD3 epsilon extracellular domain (see SEQ ID NO: 654). Even more specifically, the epitope comprises at least the amino acid sequence Gln-Asp-Gly-Asn-Glu. Callithrix jacchus is a new world primate belonging to the family of Callitrichidae, while Saimiri sciureus is a new world primate belonging to the family of Cebidae. Binders having such characteristics are described in detail in WO 2008/119567.

Antibodies or bispecific antibody constructs directed against (human) CD3 or specifically against CD3 epsilon are known in the art, and their CDRs, VH and VL sequences can serve as a basis for the second binding domain of the antibody construct of the invention. For example, Kung et al. reported in 1979 the development of OKT3 (Ortho Kung T3), the first mAb recognizing CD3 (specifically, the epsilon chain of CD3) on human T cells. OKT3 (muromonab) was the first monoclonal antibody of murine origin to become available for therapy in humans. Newer anti-CD3 monoclonal antibodies include otelixizumab (TRX4), teplizumab (MGA031), foralumab and visilizumab, all targeting the epsilon chain of CD3. Bispecific antibody constructs directed against a (cancer) target and CD3 are also being developed and (pre-)clinically tested, and their CD3 binding domain (CDRs, VH, VL) may serve as a basis for the second binding domain of the antibody construct of the invention. Examples include, but are not limited to, Blinatumomab, Solitomab (MT110, AMG 110), Catumaxomab, Duvortuxizumab, Ertumaxomab, Mosunetuzumab, FBTA05 (Bi20, TPBs05), CEA-TCB (RG7802, RO6958688), AFM11, and MGD006 (S80880). Other examples of CD3 binding domains are disclosed e.g. in U.S. Pat. Nos. 7,994,289 B2, 7,728,114 B2, 7,381,803 B1, and 6,706,265 B1.

It is envisaged for the antibody construct of the present invention that the second domain which binds to CD3 comprises a VL region comprising CDR-L1, CDR-L2 and CDR-L3 selected from:
(a) CDR-L1 as depicted in SEQ ID NO: 542, CDR-L2 as depicted in SEQ ID NO: 543, and CDR-L3 as depicted in SEQ ID NO: 544;
(b) CDR-L1 as depicted in SEQ ID NO: 599, CDR-L2 as depicted in SEQ ID NO: 600, and CDR-L3 as depicted in SEQ ID NO: 601; and
(c) CDR-L1 as depicted in SEQ ID NO: 621, CDR-L2 as depicted in SEQ ID NO: 622, and CDR-L3 as depicted in SEQ ID NO: 623.

It is also envisaged for the antibody construct of the present invention that the second domain which binds to CD3 comprises a VH region comprising CDR-H1, CDR-H2 and CDR-H3 selected from:
(a) CDR-H1 as depicted in SEQ ID NO: 534, CDR-H2 as depicted in SEQ ID NO: 535, and CDR-H3 as depicted in SEQ ID NO: 536;
(b) CDR-H1 as depicted in SEQ ID NO: 545, CDR-H2 as depicted in SEQ ID NO: 546, and CDR-H3 as depicted in SEQ ID NO: 547;
(c) CDR-H1 as depicted in SEQ ID NO: 557, CDR-H2 as depicted in SEQ ID NO: 558, and CDR-H3 as depicted in SEQ ID NO: 559;
(d) CDR-H1 as depicted in SEQ ID NO: 568, CDR-H2 as depicted in SEQ ID NO: 569, and CDR-H3 as depicted in SEQ ID NO: 570;
(e) CDR-H1 as depicted in SEQ ID NO: 579, CDR-H2 as depicted in SEQ ID NO: 580, and CDR-H3 as depicted in SEQ ID NO: 581;
(f) CDR-H1 as depicted in SEQ ID NO: 591, CDR-H2 as depicted in SEQ ID NO: 592, and CDR-H3 as depicted in SEQ ID NO: 593;
(g) CDR-H1 as depicted in SEQ ID NO: 602, CDR-H2 as depicted in SEQ ID NO: 603, and CDR-H3 as depicted in SEQ ID NO: 604;
(h) CDR-H1 as depicted in SEQ ID NO: 613, CDR-H2 as depicted in SEQ ID NO: 614, and CDR-H3 as depicted in SEQ ID NO: 615;
(i) CDR-H1 as depicted in SEQ ID NO: 624, CDR-H2 as depicted in SEQ ID NO: 625, and CDR-H3 as depicted in SEQ ID NO: 626; and
(j) CDR-H1 as depicted in SEQ ID NO: 636, CDR-H2 as depicted in SEQ ID NO: 637, and CDR-H3 as depicted in SEQ ID NO: 638.

It is furthermore envisaged for the antibody construct of the present invention that the second domain which binds to CD3 comprises a VL region comprising CDR-L1, CDR-L2 and CDR-L3 and a VH region comprising CDR-H1, CDR-H2 and CDR-H3 selected from:
(a) CDR-L1 as depicted in SEQ ID NO: 531, CDR-L2 as depicted in SEQ ID NO: 532, CDR-L3 as depicted in SEQ ID NO: 533, CDR-H1 as depicted in SEQ ID NO: 534, CDR-H2 as depicted in SEQ ID NO: 535, and CDR-H3 as depicted in SEQ ID NO: 536;
(b) CDR-L1 as depicted in SEQ ID NO: 542, CDR-L2 as depicted in SEQ ID NO: 543, CDR-L3 as depicted in SEQ ID NO: 544, CDR-H1 as depicted in SEQ ID NO: 545, CDR-H2 as depicted in SEQ ID NO: 546, and CDR-H3 as depicted in SEQ ID NO: 547;
(c) CDR-L1 as depicted in SEQ ID NO: 554, CDR-L2 as depicted in SEQ ID NO: 555, CDR-L3 as depicted in SEQ ID NO: 556, CDR-H1 as depicted in SEQ ID NO: 557, CDR-H2 as depicted in SEQ ID NO: 558, and CDR-H3 as depicted in SEQ ID NO: 559;
(d) CDR-L1 as depicted in SEQ ID NO: 565, CDR-L2 as depicted in SEQ ID NO: 566, CDR-L3 as depicted in SEQ ID NO: 567, CDR-H1 as depicted in SEQ ID NO: 568, CDR-H2 as depicted in SEQ ID NO: 569, and CDR-H3 as depicted in SEQ ID NO: 570;
(e) CDR-L1 as depicted in SEQ ID NO: 576, CDR-L2 as depicted in SEQ ID NO: 577, CDR-L3 as depicted in SEQ ID NO: 578, CDR-H1 as depicted in SEQ ID NO: 579, CDR-H2 as depicted in SEQ ID NO: 580, and CDR-H3 as depicted in SEQ ID NO: 581;
(f) CDR-L1 as depicted in SEQ ID NO: 588, CDR-L2 as depicted in SEQ ID NO: 589, CDR-L3 as depicted in SEQ ID NO: 590, CDR-H1 as depicted in SEQ ID NO: 591, CDR-H2 as depicted in SEQ ID NO: 592, and CDR-H3 as depicted in SEQ ID NO: 593;
(g) CDR-L1 as depicted in SEQ ID NO: 599, CDR-L2 as depicted in SEQ ID NO: 600, CDR-L3 as depicted in SEQ ID NO: 601, CDR-H1 as depicted in SEQ ID NO: 602, CDR-H2 as depicted in SEQ ID NO: 603, and CDR-H3 as depicted in SEQ ID NO: 604;
(h) CDR-L1 as depicted in SEQ ID NO: 610, CDR-L2 as depicted in SEQ ID NO: 611, CDR-L3 as depicted in SEQ ID NO: 612, CDR-H1 as depicted in SEQ ID NO: 613, CDR-H2 as depicted in SEQ ID NO: 614, and CDR-H3 as depicted in SEQ ID NO: 615;
(i) CDR-L1 as depicted in SEQ ID NO: 621, CDR-L2 as depicted in SEQ ID NO: 622, CDR-L3 as depicted in SEQ ID NO: 623, CDR-H1 as depicted in SEQ ID NO: 624, CDR-H2 as depicted in SEQ ID NO: 625, and CDR-H3 as depicted in SEQ ID NO: 626; and
(j) CDR-L1 as depicted in SEQ ID NO: 633, CDR-L2 as depicted in SEQ ID NO: 634, CDR-L3 as depicted in SEQ ID NO: 635, CDR-H1 as depicted in SEQ ID NO: 636, CDR-H2 as depicted in SEQ ID NO: 637, and CDR-H3 as depicted in SEQ ID NO: 638.

It is also envisaged for the antibody construct of the present invention that the second domain which binds to CD3 comprises a VL region having an amino acid sequence selected from the group consisting of those depicted in SEQ ID NO: 550, SEQ ID NO: 551, SEQ ID NO: 584, SEQ ID NO: 585, SEQ ID NO: 629 and SEQ ID NO: 630.

It is furthermore envisaged that the second domain which binds to CD3 comprises a VH region having an amino acid sequence selected from the group consisting of those depicted in SEQ ID NO: 537, SEQ ID NO: 538, SEQ ID NO: 548, SEQ ID NO: 549, SEQ ID NO: 560, SEQ ID NO: 561, SEQ ID NO: 571, SEQ ID NO: 572, SEQ ID NO: 582, SEQ ID NO: 583, SEQ ID NO: 594, SEQ ID NO: 595, SEQ ID NO: 605, SEQ ID NO: 606, SEQ ID NO: 616, SEQ ID NO: 617, SEQ ID NO: 627, SEQ ID NO: 628, SEQ ID NO: 639, SEQ ID NO: 640, and SEQ ID NO: 644.

It is also envisaged for the antibody construct of the present invention that the second domain which binds to CD3 comprises a VL region and a VH region selected from the group consisting of:
(a) a VL region as depicted in SEQ ID NO: 539 or 521 and a VH region as depicted in SEQ ID NO: 537 or 538;
(b) a VL region as depicted in SEQ ID NO: 550 or 521 and a VH region as depicted in SEQ ID NO: 548 or 549;
(c) a VL region as depicted in SEQ ID NO: 562 or 521 and a VH region as depicted in SEQ ID NO: 560 or 561;
(d) a VL region as depicted in SEQ ID NO: 573 or 521 and a VH region as depicted in SEQ ID NO: 571 or 572;
(e) a VL region as depicted in SEQ ID NO: 584 or 585 and a VH region as depicted in SEQ ID NO: 582 or 583;
(f) a VL region as depicted in SEQ ID NO: 596 or 521 and a VH region as depicted in SEQ ID NO: 594 or 595;
(g) a VL region as depicted in SEQ ID NO: 607 or 585 and a VH region as depicted in SEQ ID NO: 605 or 606;
(h) a VL region as depicted in SEQ ID NO: 618 or 521 and a VH region as depicted in SEQ ID NO: 616 or 617;
(i) a VL region as depicted in SEQ ID NO: 629 or 630 and a VH region as depicted in SEQ ID NO: 627 or 628;
(j) a VL region as depicted in SEQ ID NO: 641 or 630 and a VH region as depicted in SEQ ID NO: 639 or 640; and
(k) a VL region as depicted in SEQ ID NO: 645 and a VH region as depicted in SEQ ID NO: 644.

It is also envisaged for the antibody construct of the present invention that the second domain which binds to CD3 comprises or consists of a polypeptide having an amino acid sequence selected from the group consisting of those depicted in SEQ ID NOs: 540, 541, 552, 553, 563, 564, 574, 575, 586, 587, 597, 598, 608, 609, 619, 620, 631, 632, 642, 643, and 646.

It is also envisaged that the antibody construct of the present invention competes for binding to CD3 with:
a) an antibody or antibody construct comprising a domain which binds to CD3 on the surface of a T cell, wherein said domain comprises a VH region comprising CDR-H1 as depicted in SEQ ID NO: 636, CDR-H2 as depicted in SEQ ID NO: 637, and CDR-H3 as depicted in SEQ ID NO: 638, and a VL region comprising CDR-L1 as depicted in SEQ ID NO: 633, CDR-L2 as depicted in SEQ ID NO: 634, CDR-L3 as depicted in SEQ ID NO: 635;
b) an antibody or antibody construct comprising a domain which binds to CD3 on the surface of a T cell, wherein said domain comprises a VH region as depicted in SEQ ID NO: 639, and a VL region as depicted in SEQ ID NO: 641;
c) an antibody construct comprising a domain which binds to CD3 on the surface of a T cell, wherein said domain comprises the amino acid sequence as depicted in SEQ ID NO: 642; or
d) an antibody construct having the amino acid sequence as depicted in SEQ ID NO: 661.

It is also envisaged that the antibody construct of the present invention binds to the same epitope of CD3 as:
a) an antibody or antibody construct comprising a domain which binds to CD3 on the surface of a T cell, wherein said domain comprises a VH region comprising CDR-H1 as depicted in SEQ ID NO: 636, CDR-H2 as depicted in SEQ ID NO: 637, and CDR-H3 as depicted in SEQ ID NO: 638, and a VL region comprising CDR-L1 as depicted in SEQ ID NO: 633, CDR-L2 as depicted in SEQ ID NO: 634, CDR-L3 as depicted in SEQ ID NO: 635;
b) an antibody or antibody construct comprising a domain which binds to CD3 on the surface of a T cell, wherein said domain comprises a VH region as depicted in SEQ ID NO: 639, and a VL region as depicted in SEQ ID NO: 641;
c) an antibody construct comprising a domain which binds to CD3 on the surface of a T cell, wherein said domain comprises the amino acid sequence as depicted in SEQ ID NO: 642; or d) an antibody construct having the amino acid sequence as depicted in SEQ ID NO: 661.

It is furthermore envisaged that the antibody construct of the present invention comprises a polypeptide having an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530. It is envisaged that the antibody construct of the present invention comprises a polypeptide having an amino acid sequence as depicted in SEQ ID NO: 180.

It is also envisaged that the antibody construct of the present invention comprises or consists of a polypeptide which has an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530, and which is linked at its N-terminus or at its C-terminus with a protein purification tag, preferably via a peptide bond (amide bond). The linking of the protein purification tag at the C-terminus of the polypeptide is preferred. It is envisaged that the protein purification tag is a short peptide. For example, the length of the short peptide may be 2-30 amino acids, 4-25 amino acids, 5-20 amino acids or 6-19 amino acids. Examples of protein purification tags include, but are not limited to, AU1 epitope (e.g. as depicted in SEQ ID NO: 666), AU5 epitope (e.g. as depicted in SEQ ID NO: 667), T7-tag (e.g. as depicted in SEQ ID NO: 668), V5-tag (e.g. as depicted in SEQ ID NO: 669), B-tag (e.g. as depicted in SEQ ID NO: 670), E2 epitope (e.g. as depicted in SEQ ID NO: 671), FLAG epitope/FLAG tag (e.g. as depicted in SEQ ID NO: 672), Glu-Glu tag (e.g. as depicted in SEQ ID NOs: 673 or 674), HA tag, Histidine affinity tag (e.g. as depicted in SEQ ID NO: 675), HSV epitope (e.g. as depicted in SEQ ID NO: 676), KT3 epitope (e.g. as depicted in SEQ ID NO: 677), Myc epitope (e.g. as depicted in SEQ ID NO: 678), polyarginine tag (5-6 Arg residues), polyaspartate tag (5-16 Asp residues), polyhistidine tag (2-10 His residues, usually 6 His residues, see e.g. SEQ ID NOs: 662-665), polyphenylalanine tag (usually 11 Phe residues), S1 tag (e.g. as depicted in SEQ ID NO: 679), S-tag (e.g. as depicted in SEQ ID NO: 680), Strep-tag (e.g. as depicted in SEQ ID NOs: 681 or 682), universal tag (e.g. as depicted in SEQ ID NO: 683), VSV-G (e.g. as depicted in SEQ ID NO: 684), Protein C (e.g. as depicted in SEQ ID NO: 685), and Protein A. A histidine tag is preferred, especially a 6x His tag (SEQ ID NO: 663). Is it hence further envisaged that the antibody construct of the present invention consists of a polypeptide which has an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530, and which is linked at its C-terminus with a 6xHis tag via a peptide bond. One embodiment of the antibody construct of the present invention has an amino acid sequence as depicted in SEQ ID NO: 661.

It is also envisaged that the antibody construct of the present invention binds to the same epitope of BCMA as:
a) an antibody or antibody construct comprising a domain which binds to BCMA on the surface of a target cell, wherein said domain comprises a VH region comprising CDR-H1 as depicted in SEQ ID NO: 171, CDR-H2 as depicted in SEQ ID NO: 172, and CDR-H3 as depicted in SEQ ID NO: 173, and a VL region comprising CDR-L1 as depicted in SEQ ID NO: 174, CDR-L2 as depicted in SEQ ID NO: 175, and CDR-L3 as depicted in SEQ ID NO: 176;
b) an antibody or antibody construct comprising a domain which binds to BCMA on the surface of a target cell, wherein said domain comprises a VH region as depicted in SEQ ID NO: 177, and a VL region as depicted in SEQ ID NO: 178;
c) an antibody construct comprising a domain which binds to BCMA on the surface of a target cell, wherein said domain comprises the amino acid sequence as depicted in SEQ ID NO: 179; or
d) an antibody construct having the amino acid sequence as depicted in SEQ ID NO: 661.

Whether or not an antibody, antibody construct or binding domain binds to the same epitope of BCMA/BCMA on the surface of a target cell as another given antibody, antibody construct or binding domain can be measured by different analyses, e.g. by epitope mapping with chimeric or mutated BCMA molecules, as described in WO 2013/072406. Another possibility to identify the epitope within a target is an Alanine scanning assay (see e.g. Morrison KL & Weiss GA. Curr Opin Chem Biol. 2001 June;5(3):302-7), where each residue within the target (here: BCMA) to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired. Alanine scanning is usually accomplished by site-directed mutagenesis or randomly by creating a PCR library. Furthermore, computational methods to estimate thermodynamic parameters based on theoretical alanine substitutions have been developed. The data can be tested by IR, NMR Spectroscopy, mathematical methods, bioassays, etc. The same analysis can of course be applied for other targets such as CD3.

It is also envisaged that the antibody construct of the present invention competes for binding to BCMA with:
a) an antibody or antibody construct comprising a domain which binds to BCMA on the surface of a target cell, wherein said domain comprises a VH region comprising CDR-H1 as depicted in SEQ ID NO: 171, CDR-H2 as depicted in SEQ ID NO: 172, and CDR-H3 as depicted in SEQ ID NO: 173, and a VL region comprising CDR-L1 as depicted in SEQ ID NO: 174, CDR-L2 as depicted in SEQ ID NO: 175, and CDR-L3 as depicted in SEQ ID NO: 176;
b) an antibody or antibody construct comprising a domain which binds to BCMA on the surface of a target cell, wherein said domain comprises a VH region as depicted in SEQ ID NO: 177, and a VL region as depicted in SEQ ID NO: 178;
c) an antibody construct comprising a domain which binds to BCMA on the surface of a target cell, wherein said domain comprises the amino acid sequence as depicted in SEQ ID NO: 179; or
d) an antibody construct having the amino acid sequence as depicted in SEQ ID NO: 661.

Whether or not an antibody or antibody construct competes for binding to BCMA/BCMA on the surface of a target cell with another given antibody or antibody construct can be measured in a competition assay such as a competitive ELISA or a cell-based competition assay (using either cells that naturally express BCMA or cells that were stably or transiently transformed with BCMA). Avidin-coupled microparticles (beads) can also be used. Similar to an avidin-coated ELISA plate, when reacted with a biotinylated protein, each of these beads can be used as a substrate on which an assay can be performed. Antigen is coated onto a bead and then precoated with the first antibody. The second antibody is added, and any additional binding is determined. Read-out occurs via flow cytometry. The term “competes for binding”, in the present context, means that competition occurs between the two tested antibodies of at least 20%, at least 30%, at least 40%, at least 50%, at least 60%, at least 70%, at least 80% or at least 90%, as determined by any one of the assays disclosed above. The same analysis can of course be applied for other targets such as CD3.

The antibody construct of the present invention, or one or both of its binding domains, may be “humanized” or “human”. “Humanized” antibodies, variants or fragments thereof, antibody constructs and binding domains are based on immunoglobulins of mostly human sequences, which contain (a) minimal sequence(s) derived from non-human immunoglobulin. For the most part, humanized antibodies, variants or fragments thereof, antibody constructs and binding domains are based on human immunoglobulins (recipient antibodies) in which residues from a hypervariable region or CDR are replaced by residues from a hypervariable region or CDR of a non-human species (donor antibody) such as a rodent (e.g. mouse, hamster, rat or rabbit) having the desired specificity, affinity, capacity and/or biological activity. In some instances, Fv framework region (FR) residues of the human immunoglobulin are replaced by corresponding non-human residues. Furthermore, “humanized” antibodies, variants or fragments thereof, antibody constructs and binding domains as used herein may also comprise residues which are found neither in the recipient antibody nor the donor antibody. These modifications are made to further refine and optimize antibody performance. The humanized antibodies, variants or fragments thereof, antibody constructs and binding domains may also comprise at least a portion of an immunoglobulin constant region (such as Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992).

Human anti-mouse antibody (HAMA) responses have led the industry to prepare chimeric or otherwise humanized antibodies/antibody constructs. It is however expected that certain human anti-chimeric antibody (HACA) responses will be observed, particularly in chronic or multi-dose utilizations of an antibody or antibody construct. Thus, it would be desirable to provide antibody constructs comprising a human binding domain against BCMA and/or a human binding domain against CD3, in order to vitiate concerns and/or effects of HAMA or HACA response.

Therefore, according to one embodiment, the antibody construct, the first binding domain and/or the second binding domain are “human”. The term “human antibody”, “human antibody construct” and “human binding domain” includes antibodies, antibody constructs and binding domains, respectively, having antibody-derived regions such as variable and constant regions or domains which correspond substantially to human germline immunoglobulin sequences known in the art, including, for example, those described by Kabat et al. (1991) (loc. cit.). The human antibody constructs or binding domains of the invention may include amino acid residues not encoded by human germline immunoglobulin sequences (e.g., mutations introduced by random or site-specific mutagenesis in vitro or by somatic mutation in vivo), for example in the CDRs, and in particular in CDR3. The human antibody constructs or binding domains can have at least one, two, three, four, five, or more positions replaced with an amino acid residue that is not encoded by the human germline immunoglobulin sequence. The definition of human antibodies, antibody constructs and binding domains as used herein also contemplates fully human antibodies, antibody constructs and binding domains which include only non-artificially and/or genetically altered human sequences of antibodies as those can be derived by using technologies or systems such as the Xenomouse.

Antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. To avoid the use of rodent-derived antibody constructs, humanized or fully human antibody constructs can be generated through the introduction of human antibody function into a rodent so that the rodent produces fully human antibodies.

In some embodiments, the antibody construct of the invention is an “isolated” or “substantially pure” antibody construct. “Isolated” or “substantially pure”, when used to describe the antibody constructs disclosed herein, means an antibody construct that has been identified, separated and/or recovered from a component of its production environment. Preferably, the antibody construct is free or substantially free of association with all other components from its production environment. Contaminant components of its production environment, such as that resulting from recombinant transfected cells, are materials that could interfere with diagnostic or therapeutic uses for the antibody construct, and may include enzymes, hormones, and other proteinaceous or non-proteinaceous compounds. It is understood that the isolated or substantially pure antibody construct may constitute from 5% to 99.9% by weight of the total protein/polypeptide content in a given sample, depending on the circumstances. The desired antibody construct may be produced at a significantly higher concentration through the use of an inducible promoter or high expression promoter. The definition includes the production of an antibody construct in a wide variety of organisms and/or host cells that are known in the art. In certain embodiments, the antibody construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver staining. Usually, however, an isolated antibody construct will be prepared by at least one purification step.

According to one embodiment, the entire antibody construct and/or the binding domains are in the form of one or more polypeptides or in the form of proteins. In addition to proteinaceous parts, such polypeptides or proteins may include non-proteinaceous parts (e.g. chemical linkers or chemical cross-linking agents such as glutaraldehyde).

Peptides are short chains of amino acid monomers linked by covalent peptide (amide) bonds. Hence, peptides fall under the broad chemical classes of biological oligomers and polymers. Amino acids that are part of a peptide or polypeptide chain are termed “residues” and can be consecutively numbered. All peptides except cyclic peptides have an N-terminal residue at one end and a C-terminal residue at the other end of the peptide. An oligopeptide consists of only a few amino acids (usually between two and twenty). A polypeptide is a longer, continuous, and unbranched peptide chain. Peptides are distinguished from proteins on the basis of size, and as an arbitrary benchmark can be understood to contain approximately 50 or fewer amino acids.

Proteins consist of one or more polypeptides, usually arranged in a biologically functional way. While aspects of the lab techniques applied to peptides versus polypeptides and proteins differ (e.g., the specifics of electrophoresis, chromatography, etc.), the size boundaries that distinguish peptides from polypeptides and proteins are not absolute. Therefore, in the context of the present invention, the terms “peptide”, “polypeptide” and “protein” may be used interchangeably, and the term “polypeptide” is often preferred.

Polypeptides may further form multimers such as dimers, trimers and higher oligomers, which consist of more than one polypeptide molecule. Polypeptide molecules forming such dimers, trimers etc. may be identical or non-identical. The corresponding structures of higher order of such multimers are, consequently, termed homo-or heterodimers, homo-or heterotrimers etc. An example for a hereteromultimer is an antibody or immunoglobulin molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is accomplished e.g. by post-translational modifications like glycosylation, acetylation, phosphorylation and the like. A “peptide”, “polypeptide” or “protein” when referred to herein may also be chemically modified such as pegylated. Such modifications are well known in the art.

The antibody construct of the present invention is typically formulated in a pharmaceutical composition or a formulation. Materials of a pharmaceutical composition are usually formulated in concentrations that are acceptable for the site of administration. Formulations and compositions thus may be designed in accordance with the invention for delivery by any suitable route of administration.

As used herein, the term “pharmaceutical composition” relates to a composition which is suitable for administration to a patient, preferably a human patient. The particularly preferred pharmaceutical composition of this invention comprises one or a plurality of the antibody construct(s) of the invention, usually in a therapeutically effective amount. The pharmaceutical composition may further comprise suitable formulations of one or more (pharmaceutically effective) carriers, stabilizers, excipients, diluents, solubilizers, surfactants, emulsifiers, preservatives and/or adjuvants. Acceptable constituents of the composition are typically nontoxic to recipients at the dosages and concentrations employed. Pharmaceutical compositions of the invention include, but are not limited to, liquid, frozen, and lyophilized compositions. If the pharmaceutical composition has been lyophilized, the lyophilized material is reconstituted in an appropriate liquid prior to administration. The lyophilized material may e.g. be reconstituted in bacteriostatic water for injection (BWFI), physiological saline, phosphate buffered saline (PBS), or the same formulation the protein had been in prior to lyophilization.

The compositions may comprise a pharmaceutically acceptable carrier. In general, as used herein, “pharmaceutically acceptable carrier” means any and all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the antibody construct of the invention and further one or more excipients. Excipients can be used for a wide variety of purposes, such as adjusting physical, chemical, or biological properties of formulations, such as adjustment of viscosity, and or processes of the invention to improve effectiveness and/or to stabilize such formulations and processes against degradation and spoilage e.g. due to stresses that occur during manufacturing, shipping, storage, pre-use preparation, administration, and thereafter. Excipients should in general be used in their lowest effective concentrations.

In certain embodiments, the pharmaceutical composition may also contain formulation materials/substances for the purpose of modifying, maintaining or preserving certain characteristics of the composition such as the pH, osmolarity, viscosity, clarity, color, isotonicity, odor, sterility, stability, rate of dissolution or release, adsorption or penetration (see, Remington's Pharmaceutical Sciences, 18″ Edition, 1990, Mack Publishing Company).

The present invention refers to the following items:

Item 1. An antibody construct comprising a first domain which binds to BCMA and a second domain which binds to CD3, for use in the treatment or amelioration of a BCMA positive neoplasm, wherein the antibody construct is administered at a dose of 6.5 μg/day up to 650 μg/day in at least one cycle, wherein one cycle comprises a period of administration of the antibody construct of at least seven consecutive days.

Item 2. The antibody construct according to item 1 which is administered for 2, 3, 4, 5, 6, 7, 8, 9 or 10 cycles.

Item 3. The antibody construct according to any one of the items1 or 2, wherein one cycle comprises a period of administration of the antibody construct, followed by a period without administration of the antibody construct.

Item 4. The antibody construct according to any one of the preceding items, wherein the period of administration of the antibody construct is from one to eight weeks, preferably from two to six weeks, and more preferably from 25 to 30 days.

Item 5. The antibody construct according to item 3 or 4, wherein the period without administration of the antibody construct is at least seven consecutive days.

Item 6. The antibody construct according to any one of items 3 to 6, wherein the period without administration of the antibody construct is from one week to three months, preferably from one week to two months, and more preferably from one week to one month.

Item 7. The antibody construct according to any one of the preceding items, wherein the dose of the antibody construct is constant during each cycle.

Item 8. The antibody construct according to any one of the preceding items, wherein the dose of the antibody construct is constant during each cycle and from one cycle to the subsequent cycle.

Item 9. The antibody construct according to any one of the preceding items, which is administered parenterally, preferably intravenously, and more preferably via continuous intravenous administration.

Item 10. The antibody construct according to any one of the preceding items, wherein the BCMA positive neoplasm is selected from the group consisting of multiple myeloma, relapsed and/or refractory multiple myeloma, heavy chain multiple myeloma, light chain multiple myeloma, extramedullary myeloma, plasmacytoma, plasma cell leukemia, Waldenström's macroglobulinemia, and smoldering myeloma.

Item 11. The antibody construct according to any one of the preceding items, wherein
a) the antibody construct is a single chain polypeptide,
b) the first domain is in the format of an scFv,
c) the second domain is in the format of an scFv, and/or
d) the first domain and the second domain are connected via a linker, preferably a peptide linker, more preferably a glycine/serine linker.

Item 12. The antibody construct according to any one of the preceding items, which competes for binding to BCMA with or which binds to the same epitope of BCMA as:
a) an antibody or antibody construct comprising a domain which binds to BCMA on the surface of a target cell, wherein said domain comprises a VH region comprising CDR-H1 as depicted in SEQ ID NO: 171, CDR-H2 as depicted in SEQ ID NO: 172, and CDR-H3 as depicted in SEQ ID NO: 173, and a VL region comprising CDR-L1 as depicted in SEQ ID NO: 174, CDR-L2 as depicted in SEQ ID NO: 175, and CDR-L3 as depicted in SEQ ID NO: 176;
b) an antibody or antibody construct comprising a domain which binds to BCMA on the surface of a target cell, wherein said domain comprises a VH region as depicted in SEQ ID NO: 177, and a VL region as depicted in SEQ ID NO: 178;
c) an antibody construct comprising a domain which binds to BCMA on the surface of a target cell, wherein said domain comprises the amino acid sequence as depicted in SEQ ID NO: 179; or
d) an antibody construct having the amino acid sequence as depicted in SEQ ID NO: 661.

Item 13. The antibody construct according to any one of the preceding items, which competes for binding to CD3 with or which binds to the same epitope of CD3 as:
a) an antibody or antibody construct comprising a domain which binds to CD3 on the surface of a T cell, wherein said domain comprises a VH region comprising CDR-H1 as depicted in SEQ ID NO: 636, CDR-H2 as depicted in SEQ ID NO: 637, and CDR-H3 as depicted in SEQ ID NO: 638, and a VL region comprising CDR-L1 as depicted in SEQ ID NO: 633, CDR-L2 as depicted in SEQ ID NO: 634, CDR-L3 as depicted in SEQ ID NO: 635;
b) an antibody or antibody construct comprising a domain which binds to CD3 on the surface of a T cell, wherein said domain comprises a VH region as depicted in SEQ ID NO: 639, and a VL region as depicted in SEQ ID NO: 641;
c) an antibody construct comprising a domain which binds to CD3 on the surface of a T cell, wherein said domain comprises the amino acid sequence as depicted in SEQ ID NO: 642; or
d) an antibody construct having the amino acid sequence as depicted in SEQ ID NO: 661.

Item 14. The antibody construct according to any one of the preceding items, wherein the first domain which binds to BCMA comprises a VH region comprising CDR-H1, CDR-H2 and CDR-H3 and a VL region comprising CDR-L1, CDR-L2 and CDR-L3 selected from:
(1) CDR-H1 as depicted in SEQ ID NO: 1, CDR-H2 as depicted in SEQ ID NO: 2, CDR-H3 as depicted in SEQ ID NO: 3, CDR-L1 as depicted in SEQ ID NO: 4, CDR-L2 as depicted in SEQ ID NO: 5, and CDR-L3 as depicted in SEQ ID NO: 6;
(2) CDR-H1 as depicted in SEQ ID NO: 11, CDR-H2 as depicted in SEQ ID NO: 12, CDR-H3 as depicted in SEQ ID NO: 13, CDR-L1 as depicted in SEQ ID NO: 14, CDR-L2 as depicted in SEQ ID NO: 15, and CDR-L3 as depicted in SEQ ID NO: 16;
(3) CDR-H1 as depicted in SEQ ID NO: 21, CDR-H2 as depicted in SEQ ID NO: 22, CDR-H3 as depicted in SEQ ID NO: 23, CDR-L1 as depicted in SEQ ID NO: 24, CDR-L2 as depicted in SEQ ID NO: 25, and CDR-L3 as depicted in SEQ ID NO: 26;
(4) CDR-H1 as depicted in SEQ ID NO: 31, CDR-H2 as depicted in SEQ ID NO: 32, CDR-H3 as depicted in SEQ ID NO: 33, CDR-L1 as depicted in SEQ ID NO: 34, CDR-L2 as depicted in SEQ ID NO: 35, and CDR-L3 as depicted in SEQ ID NO: 36;
(5) CDR-H1 as depicted in SEQ ID NO: 41, CDR-H2 as depicted in SEQ ID NO: 42, CDR-H3 as depicted in SEQ ID NO: 43, CDR-L1 as depicted in SEQ ID NO: 44, CDR-L2 as depicted in SEQ ID NO: 45, and CDR-L3 as depicted in SEQ ID NO: 46;
(6) CDR-H1 as depicted in SEQ ID NO: 51, CDR-H2 as depicted in SEQ ID NO: 52, CDR-H3 as depicted in SEQ ID NO: 53, CDR-L1 as depicted in SEQ ID NO: 54, CDR-L2 as depicted in SEQ ID NO: 55, and CDR-L3 as depicted in SEQ ID NO: 56;
(7) CDR-H1 as depicted in SEQ ID NO: 61, CDR-H2 as depicted in SEQ ID NO: 62, CDR-H3 as depicted in SEQ ID NO: 63, CDR-L1 as depicted in SEQ ID NO: 64, CDR-L2 as depicted in SEQ ID NO: 65, and CDR-L3 as depicted in SEQ ID NO: 66;
(8) CDR-H1 as depicted in SEQ ID NO: 71, CDR-H2 as depicted in SEQ ID NO: 72, CDR-H3 as depicted in SEQ ID NO: 73, CDR-L1 as depicted in SEQ ID NO: 74, CDR-L2 as depicted in SEQ ID NO: 75, and CDR-L3 as depicted in SEQ ID NO: 76;
(9) CDR-H1 as depicted in SEQ ID NO: 81, CDR-H2 as depicted in SEQ ID NO: 82, CDR-H3 as depicted in SEQ ID NO: 83, CDR-L1 as depicted in SEQ ID NO: 84, CDR-L2 as depicted in SEQ ID NO: 85, and CDR-L3 as depicted in SEQ ID NO: 86;
(10) CDR-H1 as depicted in SEQ ID NO: 91, CDR-H2 as depicted in SEQ ID NO: 92, CDR-H3 as depicted in SEQ ID NO: 93, CDR-L1 as depicted in SEQ ID NO: 94, CDR-L2 as depicted in SEQ ID NO: 95, and CDR-L3 as depicted in SEQ ID NO: 96;
(11) CDR-H1 as depicted in SEQ ID NO: 101, CDR-H2 as depicted in SEQ ID NO: 102, CDR-H3 as depicted in SEQ ID NO: 103, CDR-L1 as depicted in SEQ ID NO: 104, CDR-L2 as depicted in SEQ ID NO: 105, and CDR-L3 as depicted in SEQ ID NO: 106;
(12) CDR-H1 as depicted in SEQ ID NO: 111, CDR-H2 as depicted in SEQ ID NO: 112, CDR-H3 as depicted in SEQ ID NO: 113, CDR-L1 as depicted in SEQ ID NO: 114, CDR-L2 as depicted in SEQ ID NO: 115, and CDR-L3 as depicted in SEQ ID NO: 116;
(13) CDR-H1 as depicted in SEQ ID NO: 121, CDR-H2 as depicted in SEQ ID NO: 122, CDR-H3 as depicted in SEQ ID NO: 123, CDR-L1 as depicted in SEQ ID NO: 124, CDR-L2 as depicted in SEQ ID NO: 125, and CDR-L3 as depicted in SEQ ID NO: 126;
(14) CDR-H1 as depicted in SEQ ID NO: 131, CDR-H2 as depicted in SEQ ID NO: 132, CDR-H3 as depicted in SEQ ID NO: 133, CDR-L1 as depicted in SEQ ID NO: 134, CDR-L2 as depicted in SEQ ID NO: 135, and CDR-L3 as depicted in SEQ ID NO: 136;
(15) CDR-H1 as depicted in SEQ ID NO: 141, CDR-H2 as depicted in SEQ ID NO: 142, CDR-H3 as depicted in SEQ ID NO: 143, CDR-L1 as depicted in SEQ ID NO: 144, CDR-L2 as depicted in SEQ ID NO: 145, and CDR-L3 as depicted in SEQ ID NO: 146;
(16) CDR-H1 as depicted in SEQ ID NO: 151, CDR-H2 as depicted in SEQ ID NO: 152, CDR-H3 as depicted in SEQ ID NO: 153, CDR-L1 as depicted in SEQ ID NO: 154, CDR-L2 as depicted in SEQ ID NO: 155, and CDR-L3 as depicted in SEQ ID NO: 156;
(17) CDR-H1 as depicted in SEQ ID NO: 161, CDR-H2 as depicted in SEQ ID NO: 162, CDR-H3 as depicted in SEQ ID NO: 163, CDR-L1 as depicted in SEQ ID NO: 164, CDR-L2 as depicted in SEQ ID NO: 165, and CDR-L3 as depicted in SEQ ID NO: 166;
(18) CDR-H1 as depicted in SEQ ID NO: 171, CDR-H2 as depicted in SEQ ID NO: 172, CDR-H3 as depicted in SEQ ID NO: 173, CDR-L1 as depicted in SEQ ID NO: 174, CDR-L2 as depicted in SEQ ID NO: 175, and CDR-L3 as depicted in SEQ ID NO: 176;
(19) CDR-H1 as depicted in SEQ ID NO: 181, CDR-H2 as depicted in SEQ ID NO: 182, CDR-H3 as depicted in SEQ ID NO: 183, CDR-L1 as depicted in SEQ ID NO: 184, CDR-L2 as depicted in SEQ ID NO: 185, and CDR-L3 as depicted in SEQ ID NO: 186;
(20) CDR-H1 as depicted in SEQ ID NO: 191, CDR-H2 as depicted in SEQ ID NO: 192, CDR-H3 as depicted in SEQ ID NO: 193, CDR-L1 as depicted in SEQ ID NO: 194, CDR-L2 as depicted in SEQ ID NO: 195, and CDR-L3 as depicted in SEQ ID NO: 196;
(21) CDR-H1 as depicted in SEQ ID NO: 201, CDR-H2 as depicted in SEQ ID NO: 202, CDR-H3 as depicted in SEQ ID NO: 203, CDR-L1 as depicted in SEQ ID NO: 204, CDR-L2 as depicted in SEQ ID NO: 205, and CDR-L3 as depicted in SEQ ID NO: 206;
(22) CDR-H1 as depicted in SEQ ID NO: 211, CDR-H2 as depicted in SEQ ID NO: 212, CDR-H3 as depicted in SEQ ID NO: 213, CDR-L1 as depicted in SEQ ID NO: 214, CDR-L2 as depicted in SEQ ID NO: 215, and CDR-L3 as depicted in SEQ ID NO: 216;
(23) CDR-H1 as depicted in SEQ ID NO: 221, CDR-H2 as depicted in SEQ ID NO: 222, CDR-H3 as depicted in SEQ ID NO: 223, CDR-L1 as depicted in SEQ ID NO: 224, CDR-L2 as depicted in SEQ ID NO: 225, and CDR-L3 as depicted in SEQ ID NO: 226;
(24) CDR-H1 as depicted in SEQ ID NO: 231, CDR-H2 as depicted in SEQ ID NO: 232, CDR-H3 as depicted in SEQ ID NO: 233, CDR-L1 as depicted in SEQ ID NO: 234, CDR-L2 as depicted in SEQ ID NO: 235, and CDR-L3 as depicted in SEQ ID NO: 236;
(25) CDR-H1 as depicted in SEQ ID NO: 241, CDR-H2 as depicted in SEQ ID NO: 242, CDR-H3 as depicted in SEQ ID NO: 243, CDR-L1 as depicted in SEQ ID NO: 244, CDR-L2 as depicted in SEQ ID NO: 245, and CDR-L3 as depicted in SEQ ID NO: 246;
(26) CDR-H1 as depicted in SEQ ID NO: 251, CDR-H2 as depicted in SEQ ID NO: 252, CDR-H3 as depicted in SEQ ID NO: 253, CDR-L1 as depicted in SEQ ID NO: 254, CDR-L2 as depicted in SEQ ID NO: 255, and CDR-L3 as depicted in SEQ ID NO: 256;
(27) CDR-H1 as depicted in SEQ ID NO: 261, CDR-H2 as depicted in SEQ ID NO: 262, CDR-H3 as depicted in SEQ ID NO: 263, CDR-L1 as depicted in SEQ ID NO: 264, CDR-L2 as depicted in SEQ ID NO: 265, and CDR-L3 as depicted in SEQ ID NO: 266;
(28) CDR-H1 as depicted in SEQ ID NO: 271, CDR-H2 as depicted in SEQ ID NO: 272, CDR-H3 as depicted in SEQ ID NO: 273, CDR-L1 as depicted in SEQ ID NO: 274, CDR-L2 as depicted in SEQ ID NO: 275, and CDR-L3 as depicted in SEQ ID NO: 276;
(29) CDR-H1 as depicted in SEQ ID NO: 281, CDR-H2 as depicted in SEQ ID NO: 282, CDR-H3 as depicted in SEQ ID NO: 283, CDR-L1 as depicted in SEQ ID NO: 284, CDR-L2 as depicted in SEQ ID NO: 285, and CDR-L3 as depicted in SEQ ID NO: 286;
(30) CDR-H1 as depicted in SEQ ID NO: 291, CDR-H2 as depicted in SEQ ID NO: 292, CDR-H3 as depicted in SEQ ID NO: 293, CDR-L1 as depicted in SEQ ID NO: 294, CDR-L2 as depicted in SEQ ID NO: 295, and CDR-L3 as depicted in SEQ ID NO: 296;
(31) CDR-H1 as depicted in SEQ ID NO: 301, CDR-H2 as depicted in SEQ ID NO: 302, CDR-H3 as depicted in SEQ ID NO: 303, CDR-L1 as depicted in SEQ ID NO: 304, CDR-L2 as depicted in SEQ ID NO: 305, and CDR-L3 as depicted in SEQ ID NO: 306;
(32) CDR-H1 as depicted in SEQ ID NO: 311, CDR-H2 as depicted in SEQ ID NO: 312, CDR-H3 as depicted in SEQ ID NO: 313, CDR-L1 as depicted in SEQ ID NO: 314, CDR-L2 as depicted in SEQ ID NO: 315, and CDR-L3 as depicted in SEQ ID NO: 316;
(33) CDR-H1 as depicted in SEQ ID NO: 321, CDR-H2 as depicted in SEQ ID NO: 322, CDR-H3 as depicted in SEQ ID NO: 323, CDR-L1 as depicted in SEQ ID NO: 324, CDR-L2 as depicted in SEQ ID NO: 325, and CDR-L3 as depicted in SEQ ID NO: 326;
(34) CDR-H1 as depicted in SEQ ID NO: 331, CDR-H2 as depicted in SEQ ID NO: 332, CDR-H3 as depicted in SEQ ID NO: 333, CDR-L1 as depicted in SEQ ID NO: 334, CDR-L2 as depicted in SEQ ID NO: 335, and CDR-L3 as depicted in SEQ ID NO: 336;
(35) CDR-H1 as depicted in SEQ ID NO: 341, CDR-H2 as depicted in SEQ ID NO: 342, CDR-H3 as depicted in SEQ ID NO: 343, CDR-L1 as depicted in SEQ ID NO: 344, CDR-L2 as depicted in SEQ ID NO: 345, and CDR-L3 as depicted in SEQ ID NO: 346;
(36) CDR-H1 as depicted in SEQ ID NO: 351, CDR-H2 as depicted in SEQ ID NO: 352, CDR-H3 as depicted in SEQ ID NO: 353, CDR-L1 as depicted in SEQ ID NO: 354, CDR-L2 as depicted in SEQ ID NO: 355, and CDR-L3 as depicted in SEQ ID NO: 356;
(37) CDR-H1 as depicted in SEQ ID NO: 361, CDR-H2 as depicted in SEQ ID NO: 362, CDR-H3 as depicted in SEQ ID NO: 363, CDR-L1 as depicted in SEQ ID NO: 364, CDR-L2 as depicted in SEQ ID NO: 365, and CDR-L3 as depicted in SEQ ID NO: 366;
(38) CDR-H1 as depicted in SEQ ID NO: 371, CDR-H2 as depicted in SEQ ID NO: 372, CDR-H3 as depicted in SEQ ID NO: 373, CDR-L1 as depicted in SEQ ID NO: 374, CDR-L2 as depicted in SEQ ID NO: 375, and CDR-L3 as depicted in SEQ ID NO: 376;
(39) CDR-H1 as depicted in SEQ ID NO: 381, CDR-H2 as depicted in SEQ ID NO: 382, CDR-H3 as depicted in SEQ ID NO: 383, CDR-L1 as depicted in SEQ ID NO: 384, CDR-L2 as depicted in SEQ ID NO: 385, and CDR-L3 as depicted in SEQ ID NO: 386;
(40) CDR-H1 as depicted in SEQ ID NO: 391, CDR-H2 as depicted in SEQ ID NO: 392, CDR-H3 as depicted in SEQ ID NO: 393, CDR-L1 as depicted in SEQ ID NO: 394, CDR-L2 as depicted in SEQ ID NO: 395, and CDR-L3 as depicted in SEQ ID NO: 396;
(41) CDR-H1 as depicted in SEQ ID NO: 401, CDR-H2 as depicted in SEQ ID NO: 402, CDR-H3 as depicted in SEQ ID NO: 403, CDR-L1 as depicted in SEQ ID NO: 404, CDR-L2 as depicted in SEQ ID NO: 405, and CDR-L3 as depicted in SEQ ID NO: 406;
(42) CDR-H1 as depicted in SEQ ID NO: 411, CDR-H2 as depicted in SEQ ID NO: 412, CDR-H3 as depicted in SEQ ID NO: 413, CDR-L1 as depicted in SEQ ID NO: 414, CDR-L2 as depicted in SEQ ID NO: 415, and CDR-L3 as depicted in SEQ ID NO: 416;
(43) CDR-H1 as depicted in SEQ ID NO: 421, CDR-H2 as depicted in SEQ ID NO: 422, CDR-H3 as depicted in SEQ ID NO: 423, CDR-L1 as depicted in SEQ ID NO: 424, CDR-L2 as depicted in SEQ ID NO: 425, and CDR-L3 as depicted in SEQ ID NO: 426;
(44) CDR-H1 as depicted in SEQ ID NO: 431, CDR-H2 as depicted in SEQ ID NO: 432, CDR-H3 as depicted in SEQ ID NO: 433, CDR-L1 as depicted in SEQ ID NO: 434, CDR-L2 as depicted in SEQ ID NO: 435, and CDR-L3 as depicted in SEQ ID NO: 436;
(45) CDR-H1 as depicted in SEQ ID NO: 441, CDR-H2 as depicted in SEQ ID NO: 442, CDR-H3 as depicted in SEQ ID NO: 443, CDR-L1 as depicted in SEQ ID NO: 444, CDR-L2 as depicted in SEQ ID NO: 445, and CDR-L3 as depicted in SEQ ID NO: 446;
(46) CDR-H1 as depicted in SEQ ID NO: 451, CDR-H2 as depicted in SEQ ID NO: 452, CDR-H3 as depicted in SEQ ID NO: 453, CDR-L1 as depicted in SEQ ID NO: 454, CDR-L2 as depicted in SEQ ID NO: 455, and CDR-L3 as depicted in SEQ ID NO: 456;
(47) CDR-H1 as depicted in SEQ ID NO: 461, CDR-H2 as depicted in SEQ ID NO: 462, CDR-H3 as depicted in SEQ ID NO: 463, CDR-L1 as depicted in SEQ ID NO: 464, CDR-L2 as depicted in SEQ ID NO: 465, and CDR-L3 as depicted in SEQ ID NO: 466;
(48) CDR-H1 as depicted in SEQ ID NO: 471, CDR-H2 as depicted in SEQ ID NO: 472, CDR-H3 as depicted in SEQ ID NO: 473, CDR-L1 as depicted in SEQ ID NO: 474, CDR-L2 as depicted in SEQ ID NO: 475, and CDR-L3 as depicted in SEQ ID NO: 476;
(49) CDR-H1 as depicted in SEQ ID NO: 481, CDR-H2 as depicted in SEQ ID NO: 482, CDR-H3 as depicted in SEQ ID NO: 483, CDR-L1 as depicted in SEQ ID NO: 484, CDR-L2 as depicted in SEQ ID NO: 485, and CDR-L3 as depicted in SEQ ID NO: 486;
(50) CDR-H1 as depicted in SEQ ID NO: 491, CDR-H2 as depicted in SEQ ID NO: 492, CDR-H3 as depicted in SEQ ID NO: 493, CDR-L1 as depicted in SEQ ID NO: 494, CDR-L2 as depicted in SEQ ID NO: 495, and CDR-L3 as depicted in SEQ ID NO: 496;
(51) CDR-H1 as depicted in SEQ ID NO: 501, CDR-H2 as depicted in SEQ ID NO: 502, CDR-H3 as depicted in SEQ ID NO: 503, CDR-L1 as depicted in SEQ ID NO: 504, CDR-L2 as depicted in SEQ ID NO: 505, and CDR-L3 as depicted in SEQ ID NO: 506;
(52) CDR-H1 as depicted in SEQ ID NO: 511, CDR-H2 as depicted in SEQ ID NO: 512, CDR-H3 as depicted in SEQ ID NO: 513, CDR-L1 as depicted in SEQ ID NO: 514, CDR-L2 as depicted in SEQ ID NO: 515, and CDR-L3 as depicted in SEQ ID NO: 516; and
(53) CDR-H1 as depicted in SEQ ID NO: 521, CDR-H2 as depicted in SEQ ID NO: 522, CDR-H3 as depicted in SEQ ID NO: 523, CDR-L1 as depicted in SEQ ID NO: 524, CDR-L2 as depicted in SEQ ID NO: 525, and CDR-L3 as depicted in SEQ ID NO: 526.

Item 15. The antibody construct according to any one of the preceding items, wherein the first domain which binds to BCMA comprises a VH region having an amino acid sequence selected from the group consisting of those depicted in SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 307, 317, 327, 337, 347, 357, 367, 377, 387, 397, 407, 417, 427, 437, 447, 457, 467, 477, 487, 497, 507, 517, and 527.

Item 16. The antibody construct according to any one of the preceding items, wherein the first domain which binds to BCMA comprises a VL region having an amino acid sequence selected from the group consisting of those depicted in SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, and 528.

Item 17. The antibody construct according to any one of the preceding items, wherein the first domain which binds to BCMA comprises a VH region and a VL region selected from the group consisting of:
(1) a VH region as depicted in SEQ ID NO: 7 and a VL region as depicted in SEQ ID NO: 8;
(2) a VH region as depicted in SEQ ID NO: 17 and a VL region as depicted in SEQ ID NO: 18;
(3) a VH region as depicted in SEQ ID NO: 27 and a VL region as depicted in SEQ ID NO: 28;
(4) a VH region as depicted in SEQ ID NO: 37 and a VL region as depicted in SEQ ID NO: 38;
(5) a VH region as depicted in SEQ ID NO: 47 and a VL region as depicted in SEQ ID NO: 48;
(6) a VH region as depicted in SEQ ID NO: 57 and a VL region as depicted in SEQ ID NO: 58;
(7) a VH region as depicted in SEQ ID NO: 67 and a VL region as depicted in SEQ ID NO: 68;
(8) a VH region as depicted in SEQ ID NO: 77 and a VL region as depicted in SEQ ID NO: 78;
(9) a VH region as depicted in SEQ ID NO: 87 and a VL region as depicted in SEQ ID NO: 88;
(10) a VH region as depicted in SEQ ID NO: 97 and a VL region as depicted in SEQ ID NO: 98;
(11) a VH region as depicted in SEQ ID NO: 107 and a VL region as depicted in SEQ ID NO: 108;
(12) a VH region as depicted in SEQ ID NO: 117 and a VL region as depicted in SEQ ID NO: 118;
(13) a VH region as depicted in SEQ ID NO: 127 and a VL region as depicted in SEQ ID NO: 128;
(14) a VH region as depicted in SEQ ID NO: 137 and a VL region as depicted in SEQ ID NO: 138;
(15) a VH region as depicted in SEQ ID NO: 147 and a VL region as depicted in SEQ ID NO: 148;
(16) a VH region as depicted in SEQ ID NO: 157 and a VL region as depicted in SEQ ID NO: 158;
(17) a VH region as depicted in SEQ ID NO: 167 and a VL region as depicted in SEQ ID NO: 168;
(18) a VH region as depicted in SEQ ID NO: 177 and a VL region as depicted in SEQ ID NO: 178;
(19) a VH region as depicted in SEQ ID NO: 187 and a VL region as depicted in SEQ ID NO: 188;
(20) a VH region as depicted in SEQ ID NO: 197 and a VL region as depicted in SEQ ID NO: 198;
(21) a VH region as depicted in SEQ ID NO: 207 and a VL region as depicted in SEQ ID NO: 208;
(22) a VH region as depicted in SEQ ID NO: 217 and a VL region as depicted in SEQ ID NO: 218;
(23) a VH region as depicted in SEQ ID NO: 227 and a VL region as depicted in SEQ ID NO: 228;
(24) a VH region as depicted in SEQ ID NO: 237 and a VL region as depicted in SEQ ID NO: 238;
(25) a VH region as depicted in SEQ ID NO: 247 and a VL region as depicted in SEQ ID NO: 248;
(26) a VH region as depicted in SEQ ID NO: 257 and a VL region as depicted in SEQ ID NO: 258;
(27) a VH region as depicted in SEQ ID NO: 267 and a VL region as depicted in SEQ ID NO: 268;
(28) a VH region as depicted in SEQ ID NO: 277 and a VL region as depicted in SEQ ID NO: 278;
(29) a VH region as depicted in SEQ ID NO: 287 and a VL region as depicted in SEQ ID NO: 288;
(30) a VH region as depicted in SEQ ID NO: 297 and a VL region as depicted in SEQ ID NO: 298;
(31) a VH region as depicted in SEQ ID NO: 307 and a VL region as depicted in SEQ ID NO: 308;
(32) a VH region as depicted in SEQ ID NO: 317 and a VL region as depicted in SEQ ID NO: 318;
(33) a VH region as depicted in SEQ ID NO: 327 and a VL region as depicted in SEQ ID NO: 328;
(34) a VH region as depicted in SEQ ID NO: 337 and a VL region as depicted in SEQ ID NO: 338;
(35) a VH region as depicted in SEQ ID NO: 347 and a VL region as depicted in SEQ ID NO: 348;
(36) a VH region as depicted in SEQ ID NO: 357 and a VL region as depicted in SEQ ID NO: 358;
(37) a VH region as depicted in SEQ ID NO: 367 and a VL region as depicted in SEQ ID NO: 368;
(38) a VH region as depicted in SEQ ID NO: 377 and a VL region as depicted in SEQ ID NO: 378;
(39) a VH region as depicted in SEQ ID NO: 387 and a VL region as depicted in SEQ ID NO: 388;
(40) a VH region as depicted in SEQ ID NO: 397 and a VL region as depicted in SEQ ID NO: 398;
(41) a VH region as depicted in SEQ ID NO: 407 and a VL region as depicted in SEQ ID NO: 408;
(42) a VH region as depicted in SEQ ID NO: 417 and a VL region as depicted in SEQ ID NO: 418;
(43) a VH region as depicted in SEQ ID NO: 427 and a VL region as depicted in SEQ ID NO: 428;
(44) a VH region as depicted in SEQ ID NO: 437 and a VL region as depicted in SEQ ID NO: 438;
(45) a VH region as depicted in SEQ ID NO: 447 and a VL region as depicted in SEQ ID NO: 448;
(46) a VH region as depicted in SEQ ID NO: 457 and a VL region as depicted in SEQ ID NO: 458;
(47) a VH region as depicted in SEQ ID NO: 467 and a VL region as depicted in SEQ ID NO: 468;
(48) a VH region as depicted in SEQ ID NO: 477 and a VL region as depicted in SEQ ID NO: 478;
(49) a VH region as depicted in SEQ ID NO: 487 and a VL region as depicted in SEQ ID NO: 488;
(50) a VH region as depicted in SEQ ID NO: 497 and a VL region as depicted in SEQ ID NO: 498;
(51) a VH region as depicted in SEQ ID NO: 507 and a VL region as depicted in SEQ ID NO: 508;
(52) a VH region as depicted in SEQ ID NO: 517 and a VL region as depicted in SEQ ID NO: 518; and
(53) a VH region as depicted in SEQ ID NO: 527 and a VL region as depicted in SEQ ID NO: 528.

Item 18. The antibody construct according to any one of the preceding items, wherein the first domain which binds to BCMA comprises or consists of a polypeptide having an amino acid sequence selected from the group consisting of SEQ ID NOs: 9, 19, 29, 39, 49, 59, 69, 79, 89, 109, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 519, and 529.

Item 19. The antibody construct according to any one of the preceding items, wherein the second domain which binds to CD3 comprises a VL region comprising CDR-L1, CDR-L2 and CDR-L3 selected from the group consisting of:
(a) CDR-L1 as depicted in SEQ ID NO: 542, CDR-L2 as depicted in SEQ ID NO: 543, and CDR-L3 as depicted in SEQ ID NO: 544;
(b) CDR-L1 as depicted in SEQ ID NO: 599, CDR-L2 as depicted in SEQ ID NO: 600, and CDR-L3 as depicted in SEQ ID NO: 601; and
(c) CDR-L1 as depicted in SEQ ID NO: 621, CDR-L2 as depicted in SEQ ID NO: 622, and CDR-L3 as depicted in SEQ ID NO: 623.

Item 20. The antibody construct according to any one of the preceding items, wherein the second domain which binds to CD3 comprises a VH region comprising CDR-H1, CDR-H2 and CDR-H3 selected from the group consisting of:
(a) CDR-H1 as depicted in SEQ ID NO: 534, CDR-H2 as depicted in SEQ ID NO: 535, and CDR-H3 as depicted in SEQ ID NO: 536;
(b) CDR-H1 as depicted in SEQ ID NO: 545, CDR-H2 as depicted in SEQ ID NO: 546, and CDR-H3 as depicted in SEQ ID NO: 547;
(c) CDR-H1 as depicted in SEQ ID NO: 557, CDR-H2 as depicted in SEQ ID NO: 558, and CDR-H3 as depicted in SEQ ID NO: 559;
(d) CDR-H1 as depicted in SEQ ID NO: 568, CDR-H2 as depicted in SEQ ID NO: 569, and CDR-H3 as depicted in SEQ ID NO: 570;
(e) CDR-H1 as depicted in SEQ ID NO: 579, CDR-H2 as depicted in SEQ ID NO: 580, and CDR-H3 as depicted in SEQ ID NO: 581;
(f) CDR-H1 as depicted in SEQ ID NO: 591, CDR-H2 as depicted in SEQ ID NO: 592, and CDR-H3 as depicted in SEQ ID NO: 593;
(g) CDR-H1 as depicted in SEQ ID NO: 602, CDR-H2 as depicted in SEQ ID NO: 603, and CDR-H3 as depicted in SEQ ID NO: 604;
(h) CDR-H1 as depicted in SEQ ID NO: 613, CDR-H2 as depicted in SEQ ID NO: 614, and CDR-H3 as depicted in SEQ ID NO: 615;
(i) CDR-H1 as depicted in SEQ ID NO: 624, CDR-H2 as depicted in SEQ ID NO: 625, and CDR-H3 as depicted in SEQ ID NO: 626; and
(j) CDR-H1 as depicted in SEQ ID NO: 636, CDR-H2 as depicted in SEQ ID NO: 637, and CDR-H3 as depicted in SEQ ID NO: 638.

Item 21. The antibody construct according to any one of the preceding items, wherein the second domain which binds to CD3 comprises a VL region comprising CDR-L1, CDR-L2 and CDR-L3 and a VH region comprising CDR-H1, CDR-H2 and CDR-H3 selected from the group consisting of:
(a) CDR-L1 as depicted in SEQ ID NO: 531, CDR-L2 as depicted in SEQ ID NO: 532, CDR-L3 as depicted in SEQ ID NO: 533, CDR-H1 as depicted in SEQ ID NO: 534, CDR-H2 as depicted in SEQ ID NO: 535, and CDR-H3 as depicted in SEQ ID NO: 536;
(b) CDR-L1 as depicted in SEQ ID NO: 542, CDR-L2 as depicted in SEQ ID NO: 543, CDR-L3 as depicted in SEQ ID NO: 544, CDR-H1 as depicted in SEQ ID NO: 545, CDR-H2 as depicted in SEQ ID NO: 546, and CDR-H3 as depicted in SEQ ID NO: 547;
(c) CDR-L1 as depicted in SEQ ID NO: 554, CDR-L2 as depicted in SEQ ID NO: 555, CDR-L3 as depicted in SEQ ID NO: 556, CDR-H1 as depicted in SEQ ID NO: 557, CDR-H2 as depicted in SEQ ID NO: 558, and CDR-H3 as depicted in SEQ ID NO: 559;
(d) CDR-L1 as depicted in SEQ ID NO: 565, CDR-L2 as depicted in SEQ ID NO: 566, CDR-L3 as depicted in SEQ ID NO: 567, CDR-H1 as depicted in SEQ ID NO: 568, CDR-H2 as depicted in SEQ ID NO: 569, and CDR-H3 as depicted in SEQ ID NO: 570;
(e) CDR-L1 as depicted in SEQ ID NO: 576, CDR-L2 as depicted in SEQ ID NO: 577, CDR-L3 as depicted in SEQ ID NO: 578, CDR-H1 as depicted in SEQ ID NO: 579, CDR-H2 as depicted in SEQ ID NO: 580, and CDR-H3 as depicted in SEQ ID NO: 581;
(f) CDR-L1 as depicted in SEQ ID NO: 588, CDR-L2 as depicted in SEQ ID NO: 589, CDR-L3 as depicted in SEQ ID NO: 590, CDR-H1 as depicted in SEQ ID NO: 591, CDR-H2 as depicted in SEQ ID NO: 592, and CDR-H3 as depicted in SEQ ID NO: 593;
(g) CDR-L1 as depicted in SEQ ID NO: 599, CDR-L2 as depicted in SEQ ID NO: 600, CDR-L3 as depicted in SEQ ID NO: 601, CDR-H1 as depicted in SEQ ID NO: 602, CDR-H2 as depicted in SEQ ID NO: 603, and CDR-H3 as depicted in SEQ ID NO: 604;
(h) CDR-L1 as depicted in SEQ ID NO: 610, CDR-L2 as depicted in SEQ ID NO: 611, CDR-L3 as depicted in SEQ ID NO: 612, CDR-H1 as depicted in SEQ ID NO: 613, CDR-H2 as depicted in SEQ ID NO: 614, and CDR-H3 as depicted in SEQ ID NO: 615;
(i) CDR-L1 as depicted in SEQ ID NO: 621, CDR-L2 as depicted in SEQ ID NO: 622, CDR-L3 as depicted in SEQ ID NO: 623, CDR-H1 as depicted in SEQ ID NO: 624, CDR-H2 as depicted in SEQ ID NO: 625, and CDR-H3 as depicted in SEQ ID NO: 626; and
(j) CDR-L1 as depicted in SEQ ID NO: 633, CDR-L2 as depicted in SEQ ID NO: 634, CDR-L3 as depicted in SEQ ID NO: 635, CDR-H1 as depicted in SEQ ID NO: 636, CDR-H2 as depicted in SEQ ID NO: 637, and CDR-H3 as depicted in SEQ ID NO: 638.

Item 22. The antibody construct according to any one of the preceding items, wherein the second domain which binds to CD3 comprises a VL region having an amino acid sequence selected from the group consisting of those depicted in SEQ ID NO: 550, SEQ ID NO: 551, SEQ ID NO: 584, SEQ ID NO: 585, SEQ ID NO: 629 and SEQ ID NO: 630.

Item 23. The antibody construct according to any one of the preceding items, wherein the second domain which binds to CD3 comprises a VH region having an amino acid sequence selected from the group consisting of those depicted in SEQ ID NO: 537, SEQ ID NO: 538, SEQ ID NO: 548, SEQ ID NO: 549, SEQ ID NO: 560, SEQ ID NO: 561, SEQ ID NO: 571, SEQ ID NO: 572, SEQ ID NO: 582, SEQ ID NO: 583, SEQ ID NO: 594, SEQ ID NO: 595, SEQ ID NO: 605, SEQ ID NO: 606, SEQ ID NO: 616, SEQ ID NO: 617, SEQ ID NO: 627, SEQ ID NO: 628, SEQ ID NO: 639, SEQ ID NO: 640, and SEQ ID NO: 644.
Item 24. The antibody construct according to any one of the preceding items, wherein the second domain which binds to CD3 comprises a VL region and a VH region selected from the group consisting of:
(a) a VL region as depicted in SEQ ID NO: 539 or 521 and a VH region as depicted in SEQ ID NO: 537 or 538;
(b) a VL region as depicted in SEQ ID NO: 550 or 521 and a VH region as depicted in SEQ ID NO: 548 or 549;
(c) a VL region as depicted in SEQ ID NO: 562 or 521 and a VH region as depicted in SEQ ID NO: 560 or 561;
(d) a VL region as depicted in SEQ ID NO: 573 or 521 and a VH region as depicted in SEQ ID NO: 571 or 572;
(e) a VL region as depicted in SEQ ID NO: 584 or 585 and a VH region as depicted in SEQ ID NO: 582 or 583;
(f) a VL region as depicted in SEQ ID NO: 596 or 521 and a VH region as depicted in SEQ ID NO: 594 or 595;
(g) a VL region as depicted in SEQ ID NO: 607 or 585 and a VH region as depicted in SEQ ID NO: 605 or 606;
(h) a VL region as depicted in SEQ ID NO: 618 or 521 and a VH region as depicted in SEQ ID NO: 616 or 617;
(i) a VL region as depicted in SEQ ID NO: 629 or 630 and a VH region as depicted in SEQ ID NO: 627 or 628;
(j) a VL region as depicted in SEQ ID NO: 641 or 630 and a VH region as depicted in SEQ ID NO: 639 or 640; and
(k) a VL region as depicted in SEQ ID NO: 645 and a VH region as depicted in SEQ ID NO: 644.

Item 25. The antibody construct according to any one of the preceding items, wherein the second domain which binds to CD3 comprises or consists of a polypeptide having an amino acid sequence selected from the group consisting of those depicted in SEQ ID NOs: 540, 541, 552, 553, 563, 564, 574, 575, 586, 587, 597, 598, 608, 609, 619, 620, 631, 632, 642, 643, and 646.

Item 26. The antibody construct according to any one of the preceding items, which comprises a polypeptide having an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530 and 661.

Item 27. The antibody construct according to any one of the preceding items, which has a molecular weight of about 20 to about 90 kDa, about 30 to about 80 kDa, about 40 to about 70 kDa, about 50 to about 60 kDa, about 52 to about 58 kDa, and preferably about 54 to about 56 kDa.

Item 28. The antibody construct according to any one of the preceding items, which has an elimination half-life (T_{1/2}) of about 3-36 h, about 6-30 h, or about 12-24 h.

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range. It also includes the concrete value, e.g., “about 50” includes the value “50”.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

It should be understood that the above description and the below examples provide exemplary arrangements, but the present invention is not limited to the particular methodologies, techniques, protocols, material, reagents, substances, etc., described herein and as such can vary. The terminology used herein is for the purpose of describing particular embodiments only, and is not intended to limit the scope of the present invention, which is defined solely by the claims. Aspects of the invention are provided in the independent claims. Some optional features of the invention are provided in the dependent claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended and should not be construed as to limit the scope of the present invention in any way.

### EXAMPLES

### 1. Clinical Trial Study Protocol

AMG 420 was dosed as a continuous IV (cIV) infusion for four weeks, followed by a treatment holiday of two weeks, in escalating dose levels ranging from 0.2 μg/d to 800 μg/d. Single-patient cohorts (0.2 μg/day-0.4 μg/day-0.8 μg/day-1.6 μg/day) were followed by cohorts of 3-6 patients (3.2 μg/day-6.5 μg/day-13 μg/day-25 μg/day-50 μg/day-100 μg/day-200 μg/day- 400 μg/day-800 μg/day). In total, 13 dose cohorts were completed. Treatment continued for up to five 4-weeks-on/2-weeks-off cycles; and 5 additional cycles were given in case of perceived clinical benefit. Eligible patients were ≥ 18 years old and had relapsed/refractory MM and progression after more than two prior treatment lines (including both ≥1 proteasome inhibitor and ≥1 immunomodulatory drug=IMiD). MRD response was defined for this study as <1 tumor cell/10⁴ normal cells in the bone marrow per FACS using antibodies to cytlgγ, cytlgκ, CD19, CD56 or CD138, CD38, and CD45.

### 2. Results

35 patients received AMG 420 (0.2-800 μg/d). Mean (SD) age was 63.8 (8.7) years, median age was 65 years, and min-max age was 39-79 years. 22 (63%) of the patients were male. Two patients completed 10 cycles. The 35 patients were treated for a mean (SD) of 2.3 (2.3) cycles and a median (min-max) of 1 (1-10) cycles. The treatment responders (n=8) were treated for a mean (SD) of 6.8 (4.5) cycles and a median (min-max) of 3.5 (2-10) cycles.

No anti-AMG 420 antibodies were detected up to and including 800 μg/day, and no dose-limiting toxicities (DLTs) were observed up to 400 μg/d. Two out of three patients having received 800 μg/day AMG 420 experienced DLTs: One patient having grade 3 cytokine release syndrome (CRS) within 1 day of initiating treatment with fever, hypertension, tachycardia, and retrograde amnesia (the symptoms resolved after stopping the drug); and one patient having grade 3 polyneuropathy (PPN) that required hospitalization with subsequent complete recovery. In the latter case, M protein decreased by 60% after 15 days of treatment.

Six patients had complete responses (CRs) as assessed using IMWG response criteria: one patient each at 6.5 μg/day, 100 μg/day, and 200 μg/day, and tree patients at 400 μg/d; the responses were ongoing for these last three patients (≥4.6 months). There also were two partial remissions: one partial response (PR) at 50 μg/d and a very good partial response (VGPR) at 800 μg/d. Response duration was for up to 8 cycles; 1 patient had a partial response during cycles 3-10. Four out of the six subjects with CR were MRD negative: one patient at 200 μg/day and all three patients at 400 μg/d. In an additional dose confirmation cohort (400 μg/day), two out of three patients had PRs as of cycle 1. Thus, at the dose of 400 μg/d, the objective response rate (ORR) was 5/6; and all 5 patients were responding on treatment during a period of at least two months (treatment ongoing) and up to 7 months or longer (treatment ongoing).

Following cIV infusion of AMG 420, free steady-state concentrations (Css) were reached in approximately 2 days and remained stable over the infusion period. Mean free AMG 420 Css values generally increased with increasing AMG 420 dose.

### 3. Updated Results

The following result update includes the above described results (item 2) and adds information on further patients treated with AMG 420 as described in the clinical trial study protocol (item 1). Forty-two patients received AMG 420 at 0.2-800 μg/day. Median age was 65 years and median disease duration 5.2 years. 64% of the patients were male. Patients were treated for a median (range) of 1 (1-10) cycles; responders were treated for a median (range) of 7 (1-10) cycles. Eight patients completed 5 cycles, and subsequently either completed 10 cycles (n=3), discontinued (n=3), or are still on treatment (n=2). No anti-AMG 420 antibodies were detected in any patient.

CR, PR and VGPR were assessed using IMWG response criteria. During dose escalation, responses were evident starting at 6.5 μg/day (one CR at this dose level). There was one PR at 50 μg/day, a CR at 100 μg/day, and an MRD-negative CR at 200 μg/day. At 400 μg/day, the response rate was 70% (7/10), including five MRD-negative CRs (50%), one VGPR, and one PR. Thus, at the dose of 400 μg/day, the objective response rate (ORR) was 7/10 (70%). All seven patients responded in the first cycle, some responses lasted >1 year. Responses at this dose lasted for at least a median of 9.0 months (range, 5.8-13.6+ months), with two patients ongoing on treatment. Overall, at data cutoff, six patients had MRD-negative CRs (one at 200 μg/day, five at 400 μg/day), with an additional three CRs (at 6.5, 100, and 800 μg/day), two VGPRs (at 400 and 800 μg/day), and two PRs (at 50 and 400 μg/day). The median time to any response was 1 month, with 11 of 13 responding patients (Ie, all responders receiving ≥100 μg/day) responding in the first cycle. Best responses occurred during cycle 1 (n=4), cycle 2 (n=2), cycle 3 (n=5), or in follow-up (n=2). Responses lasted for at least a median of 8.4 months (range, 2.5-15.5 months) and were over a year in three patients; responses were ongoing at last observation for 7/13 patients. For the three patients with post-treatment data, their ongoing responses lasted for up to 11 months after treatment as of last observation; in one case a patient discontinuing treatment after 2 weeks for polyneuropathy had CR nearly 9 months later. MRD-negative responses lasted for a median of 9.6 months (range, 2.8-12.8 months). Responses were seen in patients with all cytogenetic risk categories, including 5/13 responders having high-risk cytogenetics.

In this study, 800 μg/day was considered not tolerable due to grade 3 cytokine release syndrome and grade 3 polyneuropathy, both resolved. These DLTs were seen in two of three patients at 800 μg/day.

Examination of baseline cell surface BCMA expression found that BCMA was expressed on myeloma cells in all patients, with no difference in expression levels by response status. There also was no difference between responders and non-responders for percentage of plasma cells in the bone marrow that were BCMA positive or for percentage of myeloma cells in the bone marrow at baseline.

The IMWG response criteria for a complete response (see also http://imwg.myeloma.org/international-myeloma-working-group-imwg-uniform-response-criteria-for-multiple-myeloma/) are:
Negative M protein immunofixation on the serum and urine,
Disappearance of any soft tissue plasmacytomas, and
<5% plasma cells in bone marrow

The IMWG response criteria for a partial response are:
M protein electrophoresis: ≤50% reduction of serum M-protein and reduction in 24 hours urinary M-protein by ≥90% or to <200 mg/24 h
Free light chains (FLC): If the serum and urine M protein are unmeasurable, a ≥50% decrease in the difference between involved and uninvolved FLC levels is required in place of the M-protein criteria
If serum and urine M protein are not measurable, and serum free light assay is also not measurable: ≥50% reduction in plasma cells is required in place of M protein, provided baseline bone marrow plasma cell percentage was ≥30%.
In addition to the above listed criteria, if present at baseline, a ≥50% reduction in the size of soft tissue plasmacytomas is also required

The IMWG response criteria for a very good partial response are:
Serum and urine M protein detectable by immunofixation but not on electrophoresis or ≥90% reduction in serum M-protein plus urine M-protein level <100 mg/24 h

1. An antibody construct comprising a first domain which binds to BCMA and a second domain which binds to CD3, for use in the treatment or amelioration of a BCMA positive neoplasm, wherein the antibody construct is administered at a dose of 6.5 μg/day up to 650 μg/day in at least one cycle, wherein one cycle comprises a period of administration of the antibody construct of at least seven consecutive days.

2. The antibody construct according to claim 1 which is administered for 2, 3, 4, 5, 6, 7, 8, 9 or 10 cycles.

3. The antibody construct according to any one of the preceding claims, wherein one cycle comprises a period of administration of the antibody construct, followed by a period without administration of the antibody construct.

4. The antibody construct according to any one of the preceding claims, wherein the period of administration of the antibody construct is from one to eight weeks, preferably from two to six weeks, and more preferably from 25 to 30 days.

5. The antibody construct according to claim 3 or 4, wherein the period without administration of the antibody construct is at least seven consecutive days.

6. The antibody construct according to any one of claims 3 to 6, wherein the period without administration of the antibody construct is from one week to three months, preferably from one week to two months, and more preferably from one week to one month.

7. The antibody construct according to any one of the preceding claims, wherein the dose of the antibody construct is constant during each cycle.

8. The antibody construct according to any one of the preceding claims, wherein the dose of the antibody construct is constant during each cycle and from one cycle to the subsequent cycle.

9. The antibody construct according to any one of the preceding claims, which is administered parenterally, preferably intravenously, and more preferably via continuous intravenous administration.

10. The antibody construct according to any one of the preceding claims, wherein the BCMA positive neoplasm is selected from the group consisting of multiple myeloma, relapsed and/or refractory multiple myeloma, heavy chain multiple myeloma, light chain multiple myeloma, extramedullary myeloma, plasmacytoma, plasma cell leukemia, Waldenström's macroglobulinemia, and smoldering myeloma.

11. The antibody construct according to any one of the preceding claims, wherein
a) the antibody construct is a single chain polypeptide,
b) the first domain is in the format of an scFv,
c) the second domain is in the format of an scFv, and/or
d) the first domain and the second domain are connected via a linker, preferably a peptide linker, more preferably a glycine/serine linker.

12. The antibody construct according to any one of the preceding claims, which competes for binding to BCMA with or which binds to the same epitope of BCMA as:
a) an antibody or antibody construct comprising a domain which binds to BCMA on the surface of a target cell, wherein said domain comprises a VH region comprising CDR-H1 as depicted in SEQ ID NO: 171, CDR-H2 as depicted in SEQ ID NO: 172, and CDR-H3 as depicted in SEQ ID NO: 173, and a VL region comprising CDR-L1 as depicted in SEQ ID NO: 174, CDR-L2 as depicted in SEQ ID NO: 175, and CDR-L3 as depicted in SEQ ID NO: 176;
b) an antibody or antibody construct comprising a domain which binds to BCMA on the surface of a target cell, wherein said domain comprises a VH region as depicted in SEQ ID NO: 177, and a VL region as depicted in SEQ ID NO: 178;
c) an antibody construct comprising a domain which binds to BCMA on the surface of a target cell, wherein said domain comprises the amino acid sequence as depicted in SEQ ID NO: 179; or d) an antibody construct having the amino acid sequence as depicted in SEQ ID NO: 661.

13. The antibody construct according to any one of the preceding claims, which competes for binding to CD3 with or which binds to the same epitope of CD3 as:
a) an antibody or antibody construct comprising a domain which binds to CD3 on the surface of a T cell, wherein said domain comprises a VH region comprising CDR-H1 as depicted in SEQ ID NO: 636, CDR-H2 as depicted in SEQ ID NO: 637, and CDR-H3 as depicted in SEQ ID NO: 638, and a VL region comprising CDR-L1 as depicted in SEQ ID NO: 633, CDR-L2 as depicted in SEQ ID NO: 634, CDR-L3 as depicted in SEQ ID NO: 635;
b) an antibody or antibody construct comprising a domain which binds to CD3 on the surface of a T cell, wherein said domain comprises a VH region as depicted in SEQ ID NO: 639, and a VL region as depicted in SEQ ID NO: 641;
c) an antibody construct comprising a domain which binds to CD3 on the surface of a T cell, wherein said domain comprises the amino acid sequence as depicted in SEQ ID NO: 642; or
d) an antibody construct having the amino acid sequence as depicted in SEQ ID NO: 661.

14. The antibody construct according to any one of the preceding claims, wherein the first domain which binds to BCMA comprises a VH region comprising CDR-H1, CDR-H2 and CDR-H3 and a VL region comprising CDR-L1, CDR-L2 and CDR-L3 selected from:
(1) CDR-H1 as depicted in SEQ ID NO: 1, CDR-H2 as depicted in SEQ ID NO: 2, CDR-H3 as depicted in SEQ ID NO: 3, CDR-L1 as depicted in SEQ ID NO: 4, CDR-L2 as depicted in SEQ ID NO: 5, and CDR-L3 as depicted in SEQ ID NO: 6;
(2) CDR-H1 as depicted in SEQ ID NO: 11, CDR-H2 as depicted in SEQ ID NO: 12, CDR-H3 as depicted in SEQ ID NO: 13, CDR-L1 as depicted in SEQ ID NO: 14, CDR-L2 as depicted in SEQ ID NO: 15, and CDR-L3 as depicted in SEQ ID NO: 16;
(3) CDR-H1 as depicted in SEQ ID NO: 21, CDR-H2 as depicted in SEQ ID NO: 22, CDR-H3 as depicted in SEQ ID NO: 23, CDR-L1 as depicted in SEQ ID NO: 24, CDR-L2 as depicted in SEQ ID NO: 25, and CDR-L3 as depicted in SEQ ID NO: 26;
(4) CDR-H1 as depicted in SEQ ID NO: 31, CDR-H2 as depicted in SEQ ID NO: 32, CDR-H3 as depicted in SEQ ID NO: 33, CDR-L1 as depicted in SEQ ID NO: 34, CDR-L2 as depicted in SEQ ID NO: 35, and CDR-L3 as depicted in SEQ ID NO: 36;
(5) CDR-H1 as depicted in SEQ ID NO: 41, CDR-H2 as depicted in SEQ ID NO: 42, CDR-H3 as depicted in SEQ ID NO: 43, CDR-L1 as depicted in SEQ ID NO: 44, CDR-L2 as depicted in SEQ ID NO: 45, and CDR-L3 as depicted in SEQ ID NO: 46;
(6) CDR-H1 as depicted in SEQ ID NO: 51, CDR-H2 as depicted in SEQ ID NO: 52, CDR-H3 as depicted in SEQ ID NO: 53, CDR-L1 as depicted in SEQ ID NO: 54, CDR-L2 as depicted in SEQ ID NO: 55, and CDR-L3 as depicted in SEQ ID NO: 56;
(7) CDR-H1 as depicted in SEQ ID NO: 61, CDR-H2 as depicted in SEQ ID NO: 62, CDR-H3 as depicted in SEQ ID NO: 63, CDR-L1 as depicted in SEQ ID NO: 64, CDR-L2 as depicted in SEQ ID NO: 65, and CDR-L3 as depicted in SEQ ID NO: 66;
(8) CDR-H1 as depicted in SEQ ID NO: 71, CDR-H2 as depicted in SEQ ID NO: 72, CDR-H3 as depicted in SEQ ID NO: 73, CDR-L1 as depicted in SEQ ID NO: 74, CDR-L2 as depicted in SEQ ID NO: 75, and CDR-L3 as depicted in SEQ ID NO: 76;
(9) CDR-H1 as depicted in SEQ ID NO: 81, CDR-H2 as depicted in SEQ ID NO: 82, CDR-H3 as depicted in SEQ ID NO: 83, CDR-L1 as depicted in SEQ ID NO: 84, CDR-L2 as depicted in SEQ ID NO: 85, and CDR-L3 as depicted in SEQ ID NO: 86;
(10) CDR-H1 as depicted in SEQ ID NO: 91, CDR-H2 as depicted in SEQ ID NO: 92, CDR-H3 as depicted in SEQ ID NO: 93, CDR-L1 as depicted in SEQ ID NO: 94, CDR-L2 as depicted in SEQ ID NO: 95, and CDR-L3 as depicted in SEQ ID NO: 96;
(11) CDR-H1 as depicted in SEQ ID NO: 101, CDR-H2 as depicted in SEQ ID NO: 102, CDR-H3 as depicted in SEQ ID NO: 103, CDR-L1 as depicted in SEQ ID NO: 104, CDR-L2 as depicted in SEQ ID NO: 105, and CDR-L3 as depicted in SEQ ID NO: 106;
(12) CDR-H1 as depicted in SEQ ID NO: 111, CDR-H2 as depicted in SEQ ID NO: 112, CDR-H3 as depicted in SEQ ID NO: 113, CDR-L1 as depicted in SEQ ID NO: 114, CDR-L2 as depicted in SEQ ID NO: 115, and CDR-L3 as depicted in SEQ ID NO: 116;
(13) CDR-H1 as depicted in SEQ ID NO: 121, CDR-H2 as depicted in SEQ ID NO: 122, CDR-H3 as depicted in SEQ ID NO: 123, CDR-L1 as depicted in SEQ ID NO: 124, CDR-L2 as depicted in SEQ ID NO: 125, and CDR-L3 as depicted in SEQ ID NO: 126;
(14) CDR-H1 as depicted in SEQ ID NO: 131, CDR-H2 as depicted in SEQ ID NO: 132, CDR-H3 as depicted in SEQ ID NO: 133, CDR-L1 as depicted in SEQ ID NO: 134, CDR-L2 as depicted in SEQ ID NO: 135, and CDR-L3 as depicted in SEQ ID NO: 136;
(15) CDR-H1 as depicted in SEQ ID NO: 141, CDR-H2 as depicted in SEQ ID NO: 142, CDR-H3 as depicted in SEQ ID NO: 143, CDR-L1 as depicted in SEQ ID NO: 144, CDR-L2 as depicted in SEQ ID NO: 145, and CDR-L3 as depicted in SEQ ID NO: 146;
(16) CDR-H1 as depicted in SEQ ID NO: 151, CDR-H2 as depicted in SEQ ID NO: 152, CDR-H3 as depicted in SEQ ID NO: 153, CDR-L1 as depicted in SEQ ID NO: 154, CDR-L2 as depicted in SEQ ID NO: 155, and CDR-L3 as depicted in SEQ ID NO: 156;
(17) CDR-H1 as depicted in SEQ ID NO: 161, CDR-H2 as depicted in SEQ ID NO: 162, CDR-H3 as depicted in SEQ ID NO: 163, CDR-L1 as depicted in SEQ ID NO: 164, CDR-L2 as depicted in SEQ ID NO: 165, and CDR-L3 as depicted in SEQ ID NO: 166;
(18) CDR-H1 as depicted in SEQ ID NO: 171, CDR-H2 as depicted in SEQ ID NO: 172, CDR-H3 as depicted in SEQ ID NO: 173, CDR-L1 as depicted in SEQ ID NO: 174, CDR-L2 as depicted in SEQ ID NO: 175, and CDR-L3 as depicted in SEQ ID NO: 176;
(19) CDR-H1 as depicted in SEQ ID NO: 181, CDR-H2 as depicted in SEQ ID NO: 182, CDR-H3 as depicted in SEQ ID NO: 183, CDR-L1 as depicted in SEQ ID NO: 184, CDR-L2 as depicted in SEQ ID NO: 185, and CDR-L3 as depicted in SEQ ID NO: 186;
(20) CDR-H1 as depicted in SEQ ID NO: 191, CDR-H2 as depicted in SEQ ID NO: 192, CDR-H3 as depicted in SEQ ID NO: 193, CDR-L1 as depicted in SEQ ID NO: 194, CDR-L2 as depicted in SEQ ID NO: 195, and CDR-L3 as depicted in SEQ ID NO: 196;
(21) CDR-H1 as depicted in SEQ ID NO: 201, CDR-H2 as depicted in SEQ ID NO: 202, CDR-H3 as depicted in SEQ ID NO: 203, CDR-L1 as depicted in SEQ ID NO: 204, CDR-L2 as depicted in SEQ ID NO: 205, and CDR-L3 as depicted in SEQ ID NO: 206;
(22) CDR-H1 as depicted in SEQ ID NO: 211, CDR-H2 as depicted in SEQ ID NO: 212, CDR-H3 as depicted in SEQ ID NO: 213, CDR-L1 as depicted in SEQ ID NO: 214, CDR-L2 as depicted in SEQ ID NO: 215, and CDR-L3 as depicted in SEQ ID NO: 216;
(23) CDR-H1 as depicted in SEQ ID NO: 221, CDR-H2 as depicted in SEQ ID NO: 222, CDR-H3 as depicted in SEQ ID NO: 223, CDR-L1 as depicted in SEQ ID NO: 224, CDR-L2 as depicted in SEQ ID NO: 225, and CDR-L3 as depicted in SEQ ID NO: 226;
(24) CDR-H1 as depicted in SEQ ID NO: 231, CDR-H2 as depicted in SEQ ID NO: 232, CDR-H3 as depicted in SEQ ID NO: 233, CDR-L1 as depicted in SEQ ID NO: 234, CDR-L2 as depicted in SEQ ID NO: 235, and CDR-L3 as depicted in SEQ ID NO: 236;
(25) CDR-H1 as depicted in SEQ ID NO: 241, CDR-H2 as depicted in SEQ ID NO: 242, CDR-H3 as depicted in SEQ ID NO: 243, CDR-L1 as depicted in SEQ ID NO: 244, CDR-L2 as depicted in SEQ ID NO: 245, and CDR-L3 as depicted in SEQ ID NO: 246;
(26) CDR-H1 as depicted in SEQ ID NO: 251, CDR-H2 as depicted in SEQ ID NO: 252, CDR-H3 as depicted in SEQ ID NO: 253, CDR-L1 as depicted in SEQ ID NO: 254, CDR-L2 as depicted in SEQ ID NO: 255, and CDR-L3 as depicted in SEQ ID NO: 256;
(27) CDR-H1 as depicted in SEQ ID NO: 261, CDR-H2 as depicted in SEQ ID NO: 262, CDR-H3 as depicted in SEQ ID NO: 263, CDR-L1 as depicted in SEQ ID NO: 264, CDR-L2 as depicted in SEQ ID NO: 265, and CDR-L3 as depicted in SEQ ID NO: 266;
(28) CDR-H1 as depicted in SEQ ID NO: 271, CDR-H2 as depicted in SEQ ID NO: 272, CDR-H3 as depicted in SEQ ID NO: 273, CDR-L1 as depicted in SEQ ID NO: 274, CDR-L2 as depicted in SEQ ID NO: 275, and CDR-L3 as depicted in SEQ ID NO: 276;
(29) CDR-H1 as depicted in SEQ ID NO: 281, CDR-H2 as depicted in SEQ ID NO: 282, CDR-H3 as depicted in SEQ ID NO: 283, CDR-L1 as depicted in SEQ ID NO: 284, CDR-L2 as depicted in SEQ ID NO: 285, and CDR-L3 as depicted in SEQ ID NO: 286;
(30) CDR-H1 as depicted in SEQ ID NO: 291, CDR-H2 as depicted in SEQ ID NO: 292, CDR-H3 as depicted in SEQ ID NO: 293, CDR-L1 as depicted in SEQ ID NO: 294, CDR-L2 as depicted in SEQ ID NO: 295, and CDR-L3 as depicted in SEQ ID NO: 296;
(31) CDR-H1 as depicted in SEQ ID NO: 301, CDR-H2 as depicted in SEQ ID NO: 302, CDR-H3 as depicted in SEQ ID NO: 303, CDR-L1 as depicted in SEQ ID NO: 304, CDR-L2 as depicted in SEQ ID NO: 305, and CDR-L3 as depicted in SEQ ID NO: 306;
(32) CDR-H1 as depicted in SEQ ID NO: 311, CDR-H2 as depicted in SEQ ID NO: 312, CDR-H3 as depicted in SEQ ID NO: 313, CDR-L1 as depicted in SEQ ID NO: 314, CDR-L2 as depicted in SEQ ID NO: 315, and CDR-L3 as depicted in SEQ ID NO: 316;
(33) CDR-H1 as depicted in SEQ ID NO: 321, CDR-H2 as depicted in SEQ ID NO: 322, CDR-H3 as depicted in SEQ ID NO: 323, CDR-L1 as depicted in SEQ ID NO: 324, CDR-L2 as depicted in SEQ ID NO: 325, and CDR-L3 as depicted in SEQ ID NO: 326;
(34) CDR-H1 as depicted in SEQ ID NO: 331, CDR-H2 as depicted in SEQ ID NO: 332, CDR-H3 as depicted in SEQ ID NO: 333, CDR-L1 as depicted in SEQ ID NO: 334, CDR-L2 as depicted in SEQ ID NO: 335, and CDR-L3 as depicted in SEQ ID NO: 336;
(35) CDR-H1 as depicted in SEQ ID NO: 341, CDR-H2 as depicted in SEQ ID NO: 342, CDR-H3 as depicted in SEQ ID NO: 343, CDR-L1 as depicted in SEQ ID NO: 344, CDR-L2 as depicted in SEQ ID NO: 345, and CDR-L3 as depicted in SEQ ID NO: 346;
(36) CDR-H1 as depicted in SEQ ID NO: 351, CDR-H2 as depicted in SEQ ID NO: 352, CDR-H3 as depicted in SEQ ID NO: 353, CDR-L1 as depicted in SEQ ID NO: 354, CDR-L2 as depicted in SEQ ID NO: 355, and CDR-L3 as depicted in SEQ ID NO: 356;
(37) CDR-H1 as depicted in SEQ ID NO: 361, CDR-H2 as depicted in SEQ ID NO: 362, CDR-H3 as depicted in SEQ ID NO: 363, CDR-L1 as depicted in SEQ ID NO: 364, CDR-L2 as depicted in SEQ ID NO: 365, and CDR-L3 as depicted in SEQ ID NO: 366;
(38) CDR-H1 as depicted in SEQ ID NO: 371, CDR-H2 as depicted in SEQ ID NO: 372, CDR-H3 as depicted in SEQ ID NO: 373, CDR-L1 as depicted in SEQ ID NO: 374, CDR-L2 as depicted in SEQ ID NO: 375, and CDR-L3 as depicted in SEQ ID NO: 376;
(39) CDR-H1 as depicted in SEQ ID NO: 381, CDR-H2 as depicted in SEQ ID NO: 382, CDR-H3 as depicted in SEQ ID NO: 383, CDR-L1 as depicted in SEQ ID NO: 384, CDR-L2 as depicted in SEQ ID NO: 385, and CDR-L3 as depicted in SEQ ID NO: 386;
(40) CDR-H1 as depicted in SEQ ID NO: 391, CDR-H2 as depicted in SEQ ID NO: 392, CDR-H3 as depicted in SEQ ID NO: 393, CDR-L1 as depicted in SEQ ID NO: 394, CDR-L2 as depicted in SEQ ID NO: 395, and CDR-L3 as depicted in SEQ ID NO: 396;
(41) CDR-H1 as depicted in SEQ ID NO: 401, CDR-H2 as depicted in SEQ ID NO: 402, CDR-H3 as depicted in SEQ ID NO: 403, CDR-L1 as depicted in SEQ ID NO: 404, CDR-L2 as depicted in SEQ ID NO: 405, and CDR-L3 as depicted in SEQ ID NO: 406;
(42) CDR-H1 as depicted in SEQ ID NO: 411, CDR-H2 as depicted in SEQ ID NO: 412, CDR-H3 as depicted in SEQ ID NO: 413, CDR-L1 as depicted in SEQ ID NO: 414, CDR-L2 as depicted in SEQ ID NO: 415, and CDR-L3 as depicted in SEQ ID NO: 416;
(43) CDR-H1 as depicted in SEQ ID NO: 421, CDR-H2 as depicted in SEQ ID NO: 422, CDR-H3 as depicted in SEQ ID NO: 423, CDR-L1 as depicted in SEQ ID NO: 424, CDR-L2 as depicted in SEQ ID NO: 425, and CDR-L3 as depicted in SEQ ID NO: 426;
(44) CDR-H1 as depicted in SEQ ID NO: 431, CDR-H2 as depicted in SEQ ID NO: 432, CDR-H3 as depicted in SEQ ID NO: 433, CDR-L1 as depicted in SEQ ID NO: 434, CDR-L2 as depicted in SEQ ID NO: 435, and CDR-L3 as depicted in SEQ ID NO: 436;
(45) CDR-H1 as depicted in SEQ ID NO: 441, CDR-H2 as depicted in SEQ ID NO: 442, CDR-H3 as depicted in SEQ ID NO: 443, CDR-L1 as depicted in SEQ ID NO: 444, CDR-L2 as depicted in SEQ ID NO: 445, and CDR-L3 as depicted in SEQ ID NO: 446;
(46) CDR-H1 as depicted in SEQ ID NO: 451, CDR-H2 as depicted in SEQ ID NO: 452, CDR-H3 as depicted in SEQ ID NO: 453, CDR-L1 as depicted in SEQ ID NO: 454, CDR-L2 as depicted in SEQ ID NO: 455, and CDR-L3 as depicted in SEQ ID NO: 456;
(47) CDR-H1 as depicted in SEQ ID NO: 461, CDR-H2 as depicted in SEQ ID NO: 462, CDR-H3 as depicted in SEQ ID NO: 463, CDR-L1 as depicted in SEQ ID NO: 464, CDR-L2 as depicted in SEQ ID NO: 465, and CDR-L3 as depicted in SEQ ID NO: 466;
(48) CDR-H1 as depicted in SEQ ID NO: 471, CDR-H2 as depicted in SEQ ID NO: 472, CDR-H3 as depicted in SEQ ID NO: 473, CDR-L1 as depicted in SEQ ID NO: 474, CDR-L2 as depicted in SEQ ID NO: 475, and CDR-L3 as depicted in SEQ ID NO: 476;
(49) CDR-H1 as depicted in SEQ ID NO: 481, CDR-H2 as depicted in SEQ ID NO: 482, CDR-H3 as depicted in SEQ ID NO: 483, CDR-L1 as depicted in SEQ ID NO: 484, CDR-L2 as depicted in SEQ ID NO: 485, and CDR-L3 as depicted in SEQ ID NO: 486;
(50) CDR-H1 as depicted in SEQ ID NO: 491, CDR-H2 as depicted in SEQ ID NO: 492, CDR-H3 as depicted in SEQ ID NO: 493, CDR-L1 as depicted in SEQ ID NO: 494, CDR-L2 as depicted in SEQ ID NO: 495, and CDR-L3 as depicted in SEQ ID NO: 496;
(51) CDR-H1 as depicted in SEQ ID NO: 501, CDR-H2 as depicted in SEQ ID NO: 502, CDR-H3 as depicted in SEQ ID NO: 503, CDR-L1 as depicted in SEQ ID NO: 504, CDR-L2 as depicted in SEQ ID NO: 505, and CDR-L3 as depicted in SEQ ID NO: 506;
(52) CDR-H1 as depicted in SEQ ID NO: 511, CDR-H2 as depicted in SEQ ID NO: 512, CDR-H3 as depicted in SEQ ID NO: 513, CDR-L1 as depicted in SEQ ID NO: 514, CDR-L2 as depicted in SEQ ID NO: 515, and CDR-L3 as depicted in SEQ ID NO: 516; and
(53) CDR-H1 as depicted in SEQ ID NO: 521, CDR-H2 as depicted in SEQ ID NO: 522, CDR-H3 as depicted in SEQ ID NO: 523, CDR-L1 as depicted in SEQ ID NO: 524, CDR-L2 as depicted in SEQ ID NO: 525, and CDR-L3 as depicted in SEQ ID NO: 526.

15. The antibody construct according to any one of the preceding claims, wherein the first domain which binds to BCMA comprises a VH region having an amino acid sequence selected from the group consisting of those depicted in SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 307, 317, 327, 337, 347, 357, 367, 377, 387, 397, 407, 417, 427, 437, 447, 457, 467, 477, 487, 497, 507, 517, and 527.

16. The antibody construct according to any one of the preceding claims, wherein the first domain which binds to BCMA comprises a VL region having an amino acid sequence selected from the group consisting of those depicted in SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, and 528.

17. The antibody construct according to any one of the preceding claims, wherein the first domain which binds to BCMA comprises a VH region and a VL region selected from the group consisting of:
(1) a VH region as depicted in SEQ ID NO: 7 and a VL region as depicted in SEQ ID NO: 8;
(2) a VH region as depicted in SEQ ID NO: 17 and a VL region as depicted in SEQ ID NO: 18;
(3) a VH region as depicted in SEQ ID NO: 27 and a VL region as depicted in SEQ ID NO: 28;
(4) a VH region as depicted in SEQ ID NO: 37 and a VL region as depicted in SEQ ID NO: 38;
(5) a VH region as depicted in SEQ ID NO: 47 and a VL region as depicted in SEQ ID NO: 48;
(6) a VH region as depicted in SEQ ID NO: 57 and a VL region as depicted in SEQ ID NO: 58;
(7) a VH region as depicted in SEQ ID NO: 67 and a VL region as depicted in SEQ ID NO: 68;
(8) a VH region as depicted in SEQ ID NO: 77 and a VL region as depicted in SEQ ID NO: 78;
(9) a VH region as depicted in SEQ ID NO: 87 and a VL region as depicted in SEQ ID NO: 88;
(10) a VH region as depicted in SEQ ID NO: 97 and a VL region as depicted in SEQ ID NO: 98;
(11) a VH region as depicted in SEQ ID NO: 107 and a VL region as depicted in SEQ ID NO: 108;
(12) a VH region as depicted in SEQ ID NO: 117 and a VL region as depicted in SEQ ID NO: 118;
(13) a VH region as depicted in SEQ ID NO: 127 and a VL region as depicted in SEQ ID NO: 128;
(14) a VH region as depicted in SEQ ID NO: 137 and a VL region as depicted in SEQ ID NO: 138;
(15) a VH region as depicted in SEQ ID NO: 147 and a VL region as depicted in SEQ ID NO: 148;
(16) a VH region as depicted in SEQ ID NO: 157 and a VL region as depicted in SEQ ID NO: 158;
(17) a VH region as depicted in SEQ ID NO: 167 and a VL region as depicted in SEQ ID NO: 168;
(18) a VH region as depicted in SEQ ID NO: 177 and a VL region as depicted in SEQ ID NO: 178;
(19) a VH region as depicted in SEQ ID NO: 187 and a VL region as depicted in SEQ ID NO: 188;
(20) a VH region as depicted in SEQ ID NO: 197 and a VL region as depicted in SEQ ID NO: 198;
(21) a VH region as depicted in SEQ ID NO: 207 and a VL region as depicted in SEQ ID NO: 208;
(22) a VH region as depicted in SEQ ID NO: 217 and a VL region as depicted in SEQ ID NO: 218;
(23) a VH region as depicted in SEQ ID NO: 227 and a VL region as depicted in SEQ ID NO: 228;
(24) a VH region as depicted in SEQ ID NO: 237 and a VL region as depicted in SEQ ID NO: 238;
(25) a VH region as depicted in SEQ ID NO: 247 and a VL region as depicted in SEQ ID NO: 248;
(26) a VH region as depicted in SEQ ID NO: 257 and a VL region as depicted in SEQ ID NO: 258;
(27) a VH region as depicted in SEQ ID NO: 267 and a VL region as depicted in SEQ ID NO: 268;
(28) a VH region as depicted in SEQ ID NO: 277 and a VL region as depicted in SEQ ID NO: 278;
(29) a VH region as depicted in SEQ ID NO: 287 and a VL region as depicted in SEQ ID NO: 288;
(30) a VH region as depicted in SEQ ID NO: 297 and a VL region as depicted in SEQ ID NO: 298;
(31) a VH region as depicted in SEQ ID NO: 307 and a VL region as depicted in SEQ ID NO: 308;
(32) a VH region as depicted in SEQ ID NO: 317 and a VL region as depicted in SEQ ID NO: 318;
(33) a VH region as depicted in SEQ ID NO: 327 and a VL region as depicted in SEQ ID NO: 328;
(34) a VH region as depicted in SEQ ID NO: 337 and a VL region as depicted in SEQ ID NO: 338;
(35) a VH region as depicted in SEQ ID NO: 347 and a VL region as depicted in SEQ ID NO: 348;
(36) a VH region as depicted in SEQ ID NO: 357 and a VL region as depicted in SEQ ID NO: 358;
(37) a VH region as depicted in SEQ ID NO: 367 and a VL region as depicted in SEQ ID NO: 368;
(38) a VH region as depicted in SEQ ID NO: 377 and a VL region as depicted in SEQ ID NO: 378;
(39) a VH region as depicted in SEQ ID NO: 387 and a VL region as depicted in SEQ ID NO: 388;
(40) a VH region as depicted in SEQ ID NO: 397 and a VL region as depicted in SEQ ID NO: 398;
(41) a VH region as depicted in SEQ ID NO: 407 and a VL region as depicted in SEQ ID NO: 408;
(42) a VH region as depicted in SEQ ID NO: 417 and a VL region as depicted in SEQ ID NO: 418;
(43) a VH region as depicted in SEQ ID NO: 427 and a VL region as depicted in SEQ ID NO: 428;
(44) a VH region as depicted in SEQ ID NO: 437 and a VL region as depicted in SEQ ID NO: 438;
(45) a VH region as depicted in SEQ ID NO: 447 and a VL region as depicted in SEQ ID NO: 448;
(46) a VH region as depicted in SEQ ID NO: 457 and a VL region as depicted in SEQ ID NO: 458;
(47) a VH region as depicted in SEQ ID NO: 467 and a VL region as depicted in SEQ ID NO: 468;
(48) a VH region as depicted in SEQ ID NO: 477 and a VL region as depicted in SEQ ID NO: 478;
(49) a VH region as depicted in SEQ ID NO: 487 and a VL region as depicted in SEQ ID NO: 488;
(50) a VH region as depicted in SEQ ID NO: 497 and a VL region as depicted in SEQ ID NO: 498;
(51) a VH region as depicted in SEQ ID NO: 507 and a VL region as depicted in SEQ ID NO: 508;
(52) a VH region as depicted in SEQ ID NO: 517 and a VL region as depicted in SEQ ID NO: 518; and
(53) a VH region as depicted in SEQ ID NO: 527 and a VL region as depicted in SEQ ID NO: 528.

18. The antibody construct according to any one of the preceding claims, wherein the first domain which binds to BCMA comprises or consists of a polypeptide having an amino acid sequence selected from the group consisting of SEQ ID NOs: 9, 19, 29, 39, 49, 59, 69, 79, 89, 109, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 519, and 529.

19. The antibody construct according to any one of the preceding claims, wherein the second domain which binds to CD3 comprises a VL region comprising CDR-L1, CDR-L2 and CDR-L3 selected from the group consisting of:
(a) CDR-L1 as depicted in SEQ ID NO: 542, CDR-L2 as depicted in SEQ ID NO: 543, and CDR-L3 as depicted in SEQ ID NO: 544;
(b) CDR-L1 as depicted in SEQ ID NO: 599, CDR-L2 as depicted in SEQ ID NO: 600, and CDR-L3 as depicted in SEQ ID NO: 601; and
(c) CDR-L1 as depicted in SEQ ID NO: 621, CDR-L2 as depicted in SEQ ID NO: 622, and CDR-L3 as depicted in SEQ ID NO: 623.

20. The antibody construct according to any one of the preceding claims, wherein the second domain which binds to CD3 comprises a VH region comprising CDR-H1, CDR-H2 and CDR-H3 selected from the group consisting of:
(a) CDR-H1 as depicted in SEQ ID NO: 534, CDR-H2 as depicted in SEQ ID NO: 535, and CDR-H3 as depicted in SEQ ID NO: 536;
(b) CDR-H1 as depicted in SEQ ID NO: 545, CDR-H2 as depicted in SEQ ID NO: 546, and CDR-H3 as depicted in SEQ ID NO: 547;
(c) CDR-H1 as depicted in SEQ ID NO: 557, CDR-H2 as depicted in SEQ ID NO: 558, and CDR-H3 as depicted in SEQ ID NO: 559;
(d) CDR-H1 as depicted in SEQ ID NO: 568, CDR-H2 as depicted in SEQ ID NO: 569, and CDR-H3 as depicted in SEQ ID NO: 570;
(e) CDR-H1 as depicted in SEQ ID NO: 579, CDR-H2 as depicted in SEQ ID NO: 580, and CDR-H3 as depicted in SEQ ID NO: 581;
(f) CDR-H1 as depicted in SEQ ID NO: 591, CDR-H2 as depicted in SEQ ID NO: 592, and CDR-H3 as depicted in SEQ ID NO: 593;
(g) CDR-H1 as depicted in SEQ ID NO: 602, CDR-H2 as depicted in SEQ ID NO: 603, and CDR-H3 as depicted in SEQ ID NO: 604;
(h) CDR-H1 as depicted in SEQ ID NO: 613, CDR-H2 as depicted in SEQ ID NO: 614, and CDR-H3 as depicted in SEQ ID NO: 615;
(i) CDR-H1 as depicted in SEQ ID NO: 624, CDR-H2 as depicted in SEQ ID NO: 625, and CDR-H3 as depicted in SEQ ID NO: 626; and
(j) CDR-H1 as depicted in SEQ ID NO: 636, CDR-H2 as depicted in SEQ ID NO: 637, and CDR-H3 as depicted in SEQ ID NO: 638.

21. The antibody construct according to any one of the preceding claims, wherein the second domain which binds to CD3 comprises a VL region comprising CDR-L1, CDR-L2 and CDR-L3 and a VH region comprising CDR-H1, CDR-H2 and CDR-H3 selected from the group consisting of:
(a) CDR-L1 as depicted in SEQ ID NO: 531, CDR-L2 as depicted in SEQ ID NO: 532, CDR-L3 as depicted in SEQ ID NO: 533, CDR-H1 as depicted in SEQ ID NO: 534, CDR-H2 as depicted in SEQ ID NO: 535, and CDR-H3 as depicted in SEQ ID NO: 536;
(b) CDR-L1 as depicted in SEQ ID NO: 542, CDR-L2 as depicted in SEQ ID NO: 543, CDR-L3 as depicted in SEQ ID NO: 544, CDR-H1 as depicted in SEQ ID NO: 545, CDR-H2 as depicted in SEQ ID NO: 546, and CDR-H3 as depicted in SEQ ID NO: 547;
(c) CDR-L1 as depicted in SEQ ID NO: 554, CDR-L2 as depicted in SEQ ID NO: 555, CDR-L3 as depicted in SEQ ID NO: 556, CDR-H1 as depicted in SEQ ID NO: 557, CDR-H2 as depicted in SEQ ID NO: 558, and CDR-H3 as depicted in SEQ ID NO: 559;
(d) CDR-L1 as depicted in SEQ ID NO: 565, CDR-L2 as depicted in SEQ ID NO: 566, CDR-L3 as depicted in SEQ ID NO: 567, CDR-H1 as depicted in SEQ ID NO: 568, CDR-H2 as depicted in SEQ ID NO: 569, and CDR-H3 as depicted in SEQ ID NO: 570;
(e) CDR-L1 as depicted in SEQ ID NO: 576, CDR-L2 as depicted in SEQ ID NO: 577, CDR-L3 as depicted in SEQ ID NO: 578, CDR-H1 as depicted in SEQ ID NO: 579, CDR-H2 as depicted in SEQ ID NO: 580, and CDR-H3 as depicted in SEQ ID NO: 581;
(f) CDR-L1 as depicted in SEQ ID NO: 588, CDR-L2 as depicted in SEQ ID NO: 589, CDR-L3 as depicted in SEQ ID NO: 590, CDR-H1 as depicted in SEQ ID NO: 591, CDR-H2 as depicted in SEQ ID NO: 592, and CDR-H3 as depicted in SEQ ID NO: 593;
(g) CDR-L1 as depicted in SEQ ID NO: 599, CDR-L2 as depicted in SEQ ID NO: 600, CDR-L3 as depicted in SEQ ID NO: 601, CDR-H1 as depicted in SEQ ID NO: 602, CDR-H2 as depicted in SEQ ID NO: 603, and CDR-H3 as depicted in SEQ ID NO: 604;
(h) CDR-L1 as depicted in SEQ ID NO: 610, CDR-L2 as depicted in SEQ ID NO: 611, CDR-L3 as depicted in SEQ ID NO: 612, CDR-H1 as depicted in SEQ ID NO: 613, CDR-H2 as depicted in SEQ ID NO: 614, and CDR-H3 as depicted in SEQ ID NO: 615;
(i) CDR-L1 as depicted in SEQ ID NO: 621, CDR-L2 as depicted in SEQ ID NO: 622, CDR-L3 as depicted in SEQ ID NO: 623, CDR-H1 as depicted in SEQ ID NO: 624, CDR-H2 as depicted in SEQ ID NO: 625, and CDR-H3 as depicted in SEQ ID NO: 626; and
(j) CDR-L1 as depicted in SEQ ID NO: 633, CDR-L2 as depicted in SEQ ID NO: 634, CDR-L3 as depicted in SEQ ID NO: 635, CDR-H1 as depicted in SEQ ID NO: 636, CDR-H2 as depicted in SEQ ID NO: 637, and CDR-H3 as depicted in SEQ ID NO: 638.

22. The antibody construct according to any one of the preceding claims, wherein the second domain which binds to CD3 comprises a VL region having an amino acid sequence selected from the group consisting of those depicted in SEQ ID NO: 550, SEQ ID NO: 551, SEQ ID NO: 584, SEQ ID NO: 585, SEQ ID NO: 629 and SEQ ID NO: 630.

23. The antibody construct according to any one of the preceding claims, wherein the second domain which binds to CD3 comprises a VH region having an amino acid sequence selected from the group consisting of those depicted in SEQ ID NO: 537, SEQ ID NO: 538, SEQ ID NO: 548, SEQ ID NO: 549, SEQ ID NO: 560, SEQ ID NO: 561, SEQ ID NO: 571, SEQ ID NO: 572, SEQ ID NO: 582, SEQ ID NO: 583, SEQ ID NO: 594, SEQ ID NO: 595, SEQ ID NO: 605, SEQ ID NO: 606, SEQ ID NO: 616, SEQ ID NO: 617, SEQ ID NO: 627, SEQ ID NO: 628, SEQ ID NO: 639, SEQ ID NO: 640, and SEQ ID NO: 644.

24. The antibody construct according to any one of the preceding claims, wherein the second domain which binds to CD3 comprises a VL region and a VH region selected from the group consisting of:
(a) a VL region as depicted in SEQ ID NO: 539 or 521 and a VH region as depicted in SEQ ID NO: 537 or 538;
(b) a VL region as depicted in SEQ ID NO: 550 or 521 and a VH region as depicted in SEQ ID NO: 548 or 549;
(c) a VL region as depicted in SEQ ID NO: 562 or 521 and a VH region as depicted in SEQ ID NO: 560 or 561;
(d) a VL region as depicted in SEQ ID NO: 573 or 521 and a VH region as depicted in SEQ ID NO: 571 or 572;
(e) a VL region as depicted in SEQ ID NO: 584 or 585 and a VH region as depicted in SEQ ID NO: 582 or 583;
(f) a VL region as depicted in SEQ ID NO: 596 or 521 and a VH region as depicted in SEQ ID NO: 594 or 595;
(g) a VL region as depicted in SEQ ID NO: 607 or 585 and a VH region as depicted in SEQ ID NO: 605 or 606;
(h) a VL region as depicted in SEQ ID NO: 618 or 521 and a VH region as depicted in SEQ ID NO: 616 or 617;
(i) a VL region as depicted in SEQ ID NO: 629 or 630 and a VH region as depicted in SEQ ID NO: 627 or 628;
(j) a VL region as depicted in SEQ ID NO: 641 or 630 and a VH region as depicted in SEQ ID NO: 639 or 640; and
(k) a VL region as depicted in SEQ ID NO: 645 and a VH region as depicted in SEQ ID NO: 644.

25. The antibody construct according to any one of the preceding claims, wherein the second domain which binds to CD3 comprises or consists of a polypeptide having an amino acid sequence selected from the group consisting of those depicted in SEQ ID NOs: 540, 541, 552, 553, 563, 564, 574, 575, 586, 587, 597, 598, 608, 609, 619, 620, 631, 632, 642, 643, and 646.

26. The antibody construct according to any one of the preceding claims, which comprises a polypeptide having an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530 and 661.
